(19) United States
(12) Patent Application Publication
(10) Pub. No.: US 2022/0265558 A1
(43) Pub. Date: Aug. 25, 2022

(54) METHODS AND ASSEMBLIES FOR PREPARING AND DISPENSING LYOSPHERES OF PHARMACEUTICAL COMPOSITIONS

(71) Applicants: Akhilesh | Bhambhani, West Point, PA (US); Morrisa Jones, Upper Darby, PA (US); Derrick M. Smith, West Point, PA (US); David S. Thiriot, West Point, PA (US); Joseph M. Rock, West Point, PA (US); Merck Sharp & Dohme Corp., Rahway, NJ (US)

(72) Inventors: Akhilesh Bhambhani, Foster City, CA (US); Morrisa Jones, Upper Darby, PA (US); Derrick M. Smith, Newtown Square, PA (US); David S. Thiriot, Harleysville, PA (US); Joseph M. Rock, East Greenville, PA (US)

(73) Assignee: Merck Sharp & Dohme Corp., Rahway, NJ (US)

(21) Appl. No.: 17/628,711

(22) PCT Filed: Jul. 23, 2020
(86) PCT No.: PCT/US2020/043290

§ 371 Date: Jan. 20, 2022

Related U.S. Application Data
(60) Provisional application No. 62/878,802, filed on Jul. 26, 2019.

(51) Int. Cl. A61K 9/19; F26B 25/06; F26B 5/06; A61J 3/00

(52) U.S. Cl. CPC A61K 9/19; F26B 25/063; F26B 5/06; A61J 3/00

(57) ABSTRACT

Disclosed herein are methods for preparing and/or dispensing lyospheres of pharmaceutical compositions of biologics (e.g., vaccines, therapeutic proteins such as monoclonal antibodies) or small molecules (e.g., chemical compounds). Also described are assemblies and systems for preparing and/or dispensing the lyospheres.

### CROSS REFERENCE TO RELATED APPLICATIONS

This application claims the benefit of priority to U.S. Provisional Application No. 62/878,802, filed Jul. 26, 2019, the disclosure of which is incorporated by reference in its entirety.

### FIELD

The present invention relates to methods and assemblies for preparing and/or dispensing lyospheres of pharmaceutical compositions of biologics (e.g., vaccines, therapeutic proteins such as monoclonal antibodies) or small molecules (e.g., chemical compounds).

### BACKGROUND

Pharmaceutical compositions of biologics (e.g., vaccines, therapeutic proteins such as monoclonal antibodies) or small molecules (e.g., chemical compound) are frequently preserved by lyophilizing aliquots of a liquid composition containing the biological or chemical materials.

Methods of lyophilizing pharmaceutical compositions in the form of substantially spherically or half-spherically shaped pellets, i.e., lyospheres or lyobeads, have been described. In these methods, individual samples of the material are frozen and dried prior to placing a desired number of the dried samples into a storage container such as a glass vial. Historically, these methods relied on either (a) dispensing an aliquot of a liquid composition containing the desired amount of a material into a container of a cryogen such as liquid nitrogen, which results in direct contact of the material with the cryogen and/or (b) dispensing an aliquot of a liquid composition containing the material onto a chilled plate which constitutes the top surface of a heat sink. After the aliquots freeze on the plate, an automated system is often used for detachment of the frozen pellets from the plate. Notably, the relative position of the frozen pellets to each other is not preserved as they are removed from the freezing plate and transferred into a different container for lyophilization. In a disordered, bulk state, this present a significant challenge after lyophilization to singulate the lyospheres and dispense them into final containers.

Thus, there is a need to develop a simpler, more efficient, and more effective method for preparing, handling, and/or dispensing lyospheres of pharmaceutical compositions.

### SUMMARY

This disclosure includes methods for preparing and/or dispensing lyospheres of pharmaceutical compositions comprising, for example, biologics (e.g., vaccines, therapeutic proteins such as monoclonal antibodies), small molecules (e.g., chemical compounds), or combinations thereof, as well as assemblies and systems for preparing and/or dispensing such lyospheres.

In one aspect, provided herein is a method for freezing droplets of a pharmaceutical composition, comprising:
(a) providing an assembly comprising a generally planar base plate, wherein the base plate is placed on top of a heat sink and chilled to a low temperature, wherein the base plate is not physically attached to the heat sink; and
(b) dispensing droplets of the pharmaceutical composition on the base plate in an array format, wherein the droplets freeze on the base plate.

In certain embodiments, the assembly further comprises an insert plate overlaying the base plate; wherein the insert plate has an array of apertures; and the droplets of the pharmaceutical composition are dispensed into the apertures to be supported by the base plate.

In other embodiments, the assembly further comprises an insert plate overlaying the base plate; wherein the insert plate has an array of apertures; wherein the base plate has an array of openings and solid portions located between and surrounding the openings; wherein the apertures align with the solid portions of the base plate with no overlap with the openings; and the droplets of the pharmaceutical composition are dispensed into the apertures to be supported by the solid portions of the base plate.

In another aspect, provided herein is a method of preparing lyospheres of a pharmaceutical composition, comprising:
(a) providing an assembly comprising a generally planar base plate, wherein the base plate is placed on top of a heat sink and chilled to a low temperature, wherein the base plate is not physically attached to the heat sink;
(b) dispensing droplets of the pharmaceutical composition on the base plate in an array format, wherein the droplets freeze on the base plate; and
(c) placing the assembly in a lyophilizer to dry the frozen droplets and produce an array of lyospheres.

In some embodiments, the method of preparing lyospheres of a pharmaceutical composition comprises repeating steps (a) and (b) multiple times, preparing a stack of assemblies with a thermally conductive path formed between the assemblies, and in step (c) drying the frozen droplets in the entire stack in a lyophilizer to produce arrays of lyospheres.

In one embodiment, the thermally conductive path is formed by stacking a plurality of assemblies on top of each other, wherein each base plate has at least two raised edges, and wherein the raised edges of the base plates are in physical contact. In certain embodiments of this method, in step (c) the lowest base plate is not in full contact with a shelf of the lyophilizer.

In another embodiment, the thermally conductive path is formed by placing thermally conductive spacers along at least two edges of each base plate and stacking a plurality of assemblies on top of each other, wherein the spacers and the base plates are in physical contact. In certain embodiments of this method, in step (c) the lowest base plate is not in full contact with a shelf of the lyophilizer.

In yet another embodiment, the thermally conductive path is formed by providing a thermally conductive rack with multiple levels and placing a plurality of assemblies on the levels of the rack, wherein the base plates and the levels of the rack are in physical contact. In certain embodiments of this method, in step (c) the lowest base plate is not in full contact with a shelf of the lyophilizer.

In still another embodiment, the thermally conductive path is formed by edge mounting two thermally conductive clips to the base plate and the insert plate with the insert plate overlaying the base plate and stacking a plurality of assemblies on top of each other, wherein the clips are in physical contact. In certain embodiments of this method, in step (c) the lowest base plate is not in full contact with a shelf of the lyophilizer.

In certain embodiments, the assembly further comprises an insert plate overlaying the base plate; wherein the insert plate has an array of apertures; and wherein each droplet is dispensed into an aperture to be supported by the base plate.

In other embodiments, the assembly further comprises an insert plate overlaying the base plate; wherein the insert plate has an array of apertures; wherein the base plate has an array of openings and solid portions located between and surrounding the openings; wherein the apertures align with the solid portions of the base plate with no overlap with the openings; and wherein each droplet is dispensed into an aperture to be supported by the solid portions of the base plate.

In yet another aspect, provided herein is a method of preparing lyospheres of a pharmaceutical composition, comprising:
(a) providing an assembly comprising an insert plate overlaying a base plate; wherein the base plate has an array of openings and solid portions located between and surrounding the openings; wherein the insert plate has an array of apertures; wherein the insert plate is axially shiftable relative to the base plate from a first state to a second state; wherein in the first state, the apertures align with the solid portions of the base plate with no overlap with the openings, and in the second state, the apertures at least partially align with the openings so as to at least partially overlap with the openings; wherein the base plate is chilled to a low temperature;
(b) while the insert plate is in the first state, dispensing droplets of the pharmaceutical composition into the apertures to be supported by the solid portions of the base plate, wherein the droplets freeze on the base plate;
(c) placing the assembly in a lyophilizer to dry the frozen droplets and produce lyospheres;
(d) providing a container nest comprising an array of pharmaceutically acceptable containers, each of the containers having a loading opening, wherein the openings in the base plate align with the loading openings of the containers; and
(e) dispensing the lyospheres into the containers by axially shifting the insert plate relative to the base plate to the second state.

In some embodiments, the method of preparing lyospheres of a pharmaceutical composition comprises repeating steps (a)-(b) multiple times, preparing a stack of assemblies with a thermally conductive path formed between the assemblies; in step (c) drying the frozen droplets in the entire stack in a lyophilizer to produce lyospheres; and repeating steps (d)-(e) multiple times to dispense the lyospheres in each assembly into the containers in a container nest.

In one embodiment, the thermally conductive path is formed by stacking a plurality of assemblies on top of each other, wherein each base plate has at least two raised edges, and wherein the raised edges of the base plates are in physical contact. In certain embodiments of this method, in step (c) the lowest base plate is not in full contact with a shelf of the lyophilizer.

In another embodiment, the thermally conductive path is formed by placing thermally conductive spacers along at least two edges of each base plate and stacking a plurality of assemblies on top of each other, wherein the spacers and the base plates are in physical contact. In certain embodiments of this method, in step (c) the lowest base plate is not in full contact with a shelf of the lyophilizer.

In yet another embodiment, the thermally conductive path is formed by providing a thermally conductive rack with multiple levels and placing a plurality of assemblies on the levels of the rack, wherein the base plates and the levels of the rack are in physical contact. In certain embodiments of this method, in step (c) the lowest base plate is not in full contact with a shelf of the lyophilizer.

In still another embodiment, the thermally conductive path is formed by edge mounting two thermally conductive clips to the base plate and the insert plate with the insert plate overlaying the base plate and stacking a plurality of assemblies on top of each other, wherein the clips are in physical contact. In certain embodiments of this method, in step (c) the lowest base plate is not in full contact with a shelf of the lyophilizer.

In still another aspect, provided herein is a method for preparing lyospheres of a pharmaceutical composition, comprising:
(a) providing an assembly comprising an insert plate overlaying a base plate; wherein the base plate has an array of openings and solid portions located between and surrounding the openings; wherein the insert plate has an array of apertures; wherein the insert plate is axially shiftable relative to the base plate from a first state to a second state; wherein in the first state, the apertures align with the solid portions of the base plate with no overlap with the openings, and in the second state, the apertures at least partially align with the openings so as to at least partially overlap with the openings; wherein the base plate is chilled to a low temperature;
(b) while the insert plate is in the first state, dispensing droplets of the pharmaceutical composition into the apertures to be supported by the solid portions of the base plate, wherein the droplets freeze on the base plate;
(c) placing the assembly in a lyophilizer to dry the frozen droplets and produce lyospheres;
(d) providing a container nest comprising an array of pharmaceutically acceptable containers, each of the containers having a loading opening;
(e) providing a dispensing funnel on top of the container nest, wherein the dispensing funnel has a support plate with an array of fill openings formed therethrough, wherein a first of the fill openings extends between a first top opening, formed in a top face of the support plate, to a bottom opening, formed in a bottom face of the support plate, wherein the top openings align with the openings in the base plate, wherein the bottom openings align with the loading openings of the containers; and
(f) dispensing the lyospheres into the containers by axially shifting the insert plate relative to the base plate to the second state.

In certain embodiments of the above method, wherein in (e) a second of the fill openings extends from a second top opening, formed in the top face of the support plate, to the first fill opening so as to merge therewith.

In some embodiments, the above method for preparing lyospheres of a pharmaceutical composition comprises: repeating steps (a)-(b) multiple times, preparing a stack of assemblies with a thermally conductive path formed between the assemblies; in step (c) drying the frozen droplets in the entire stack in a lyophilizer to produce lyospheres; then repeating steps (d)-(f) multiple times to dispense the lyospheres in each assembly into the containers in a container nest.

In one embodiment, the thermally conductive path is formed by stacking a plurality of assemblies on top of each other, wherein each base plate has at least two raised edges, and wherein the raised edges of the base plates are in physical contact. In certain embodiments, in step (c) the lowest base plate is not in full contact with a shelf of the lyophilizer.

In another embodiment, the thermally conductive path is formed by placing thermally conductive spacers along at least two edges of each base plate and stacking a plurality of assemblies on top of each other, wherein the spacers and the base plates are in physical contact. In certain embodiments, in step (c) the lowest base plate is not in full contact with a shelf of the lyophilizer.

In yet another embodiment, the thermally conductive path is formed by providing a thermally conductive rack with multiple levels and placing a plurality of assemblies on the levels of the rack, wherein the base plates and the levels of the rack are in physical contact. In certain embodiments, in step (c) the lowest base plate is not in full contact with a shelf of the lyophilizer.

In still another embodiment, the thermally conductive path is formed by edge mounting two thermally conductive clips to the base plate and the insert plate with the insert plate overlaying the base plate and stacking a plurality of assemblies on top of each other, wherein the clips are in physical contact. In certain embodiments, in step (c) the lowest base plate is not in full contact with a shelf of the lyophilizer.

In yet still another aspect, provided herein is a method for preparing lyospheres of a pharmaceutical composition comprising more than one formulation, comprising:
(a) providing an assembly comprising an insert plate overlaying a base plate; wherein the base plate has an array of openings and solid portions located between and surrounding the openings; wherein the insert plate has an array of apertures; wherein the insert plate is axially shiftable relative to the base plate from a first state to a second state; wherein in the first state, the apertures align with the solid portions of the base plate with no overlap with the openings, and in the second state, the apertures at least partially align with the openings so as to at least partially overlap with the openings; wherein the base plate is chilled to a low temperature;
(b) while the insert plate is in the first state, dispensing droplets of a first formulation into a first row of the apertures and droplets of a second formulation into a second row of the apertures to be supported by the solid portions of the base plate, wherein the droplets freeze on the base plate;
(c) placing the assembly in a lyophilizer to dry the frozen droplets and produce lyospheres;
(d) providing a container nest comprising an array of pharmaceutically acceptable containers, each of the containers having a loading opening;
(e) providing a dispensing funnel on top of the container nest, wherein the dispensing funnel has a support plate with an array of fill openings formed therethrough, wherein a first of the fill openings extends between a first top opening, formed in a top face of the support plate, to a bottom opening, formed in a bottom face of the support plate, wherein a second of the fill openings extends from a second top opening, formed in the top face of the support plate, to the first fill opening so as to merge therewith, wherein the first top openings align with a first row of the openings in the base plate and the second top openings align with a second row of the openings in the base plate, wherein the bottom openings align with the loading openings of the containers; and
(f) dispensing the lyospheres into the containers by axially shifting the insert plate relative to the base plate to the second state so that the first row of the apertures at least partially align with the first row of the openings so as to at least partially overlap with the first row of the openings and the second row of the apertures at least partially align with the second row of the openings so as to at least partially overlap with the second row of the openings.

In some embodiments, the method for preparing lyospheres of a pharmaceutical composition comprising more than one formulation, comprises: repeating steps (a)-(b) multiple times, preparing a stack of assemblies with a thermally conductive path formed between the assemblies; in step (c) drying the frozen droplets in the entire stack in a lyophilizer to produce lyospheres; then repeating steps (d)-(f) multiple times to dispense the lyospheres in each assembly into the containers in a container nest.

In one embodiment, the thermally conductive path is formed by stacking a plurality of assemblies on top of each other, wherein each base plate has at least two raised edges, and wherein the raised edges of the base plates are in physical contact. In certain embodiments, in step (c) the lowest base plate is not in full contact with a shelf of the lyophilizer.

In another embodiment, the thermally conductive path is formed by placing thermally conductive spacers along at least two edges of each base plate and stacking a plurality of assemblies on top of each other, wherein the spacers and the base plates are in physical contact. In certain embodiments, in step (c) the lowest base plate is not in full contact with a shelf of the lyophilizer.

In yet another embodiment, the thermally conductive path is formed by providing a thermally conductive rack with multiple levels and placing a plurality of assemblies on the levels of the rack, wherein the base plates and the levels of the rack are in physical contact. In certain embodiments, in step (c) the lowest base plate is not in full contact with a shelf of the lyophilizer.

In still another embodiment, the thermally conductive path is formed by edge mounting two thermally conductive clips to the base plate and the insert plate with the insert plate overlaying the base plate and stacking a plurality of assemblies on top of each other, wherein the clips are in physical contact. In certain embodiments, in step (c) the lowest base plate is not in full contact with a shelf of the lyophilizer.

In some embodiments of the method for preparing lyospheres of a pharmaceutical composition comprising more than one formulation, the first formulation is an active pharmaceutical ingredient (API) formulation and the second formulation is an adjuvant formulation.

In other embodiments of the method for preparing lyospheres of a pharmaceutical composition comprising more than one formulation, wherein the first formulation is a first API formulation and the second formulation is a second API formulation.

In certain embodiments of various methods disclosed herein, the droplets of the pharmaceutical composition are dispensed at a speed of: from about 0.5 mL/min to about 75 mL/min, from about 0.5 mL/min to about 50 mL/min, from about 5 mL/min to about 50 mL/min, from about 5 mL/min to about 40 mL/min, from about 10 mL/min to about 40 mL/min, or from about 10 mL/min to about 30 mL/min.

In some embodiments of various methods disclosed herein, the droplet is about 10, 15, 20, 25, 30, 40, 50, 75, 100, 125, 150, 175, 200, 225, or 250 μL.

In other embodiments of various methods disclosed herein, the droplets of the pharmaceutical composition are dispensed through a dispensing tip; wherein the distance from the bottom of the dispensing tip to the base plate is: from about 0.05 cm to about 1 cm, from about 0.05 cm to about 0.8 cm, from about 0.05 cm to about 0.5 cm, from about 0.05 cm to about 0.3 cm, or from about 0.1 cm to about 0.3 cm.

In yet other embodiments of various methods disclosed herein, the temperature of the base plate at the droplet dispense and freezing step is: from about −70° C. to about −196° C., from about −70° C. to about −150° C., from about −90° C. to about −196° C., from about −150° C. to about −196° C., from about −180° C. to about −196° C., or from about −180° C. to about −273° C.

In still other embodiments of various methods disclosed herein, the pharmaceutical composition comprises a drug substance, a chemical compound, a therapeutic protein, an antibody, a vaccine, a fusion protein, a polypeptide, a peptide, a polynucleotide, a nucleotide, an antisense RNA, a small interfering RNA (siRNA), an oncolytic virus, a diagnostic, an enzyme, an adjuvant, an antigen, a virus, a virus-like particle, a prodrug, a toxoid, a vitamin, a lipid, a lipid nanoparticle, or a combination thereof.

In yet another aspect, the various methods provided herein can be used for preparing a combination of lyospheres of different pharmaceutical compositions by dispensing a lyosphere of each pharmaceutical composition into one pharmaceutically acceptable container.

In yet still another aspect, provided herein is a container containing a lyosphere of a pharmaceutical composition, wherein the lyosphere is prepared and/or dispensed by various methods described herein.

In yet another aspect, provided herein is an assembly for preparing and/or dispensing lyospheres, the assembly comprising:

a base plate having a generally planar base with an array of openings formed therethrough, solid portions of the base being located between, and surrounding, the openings; and,

an insert plate for overlaying the base plate, the insert plate having a generally planar body with an array of apertures formed therethrough, the insert plate being axially shiftable relative to the base plate from a first state to a second state, wherein, the array of apertures is configured such that, in the first state, the apertures are aligned with the solid portions of the base plate with no overlap with the openings, and, in the second state, the apertures are at least partially aligned with the openings so as to at least partially overlap the openings.

In some embodiments, the base plate includes spaced-apart first and second side edges which extend between spaced-apart first and second ends, optionally, the first and second side edges each being greater in length than each of the first and second ends.

In further embodiments, the base plate further comprises a first upstanding channel extending along the first side edge and a second upstanding channel extending along the second side edge, the first and second channels being configured to receive the insert plate in sliding engagement to guide the insert plate during the axial shifting of the insert plate relative to the base plate.

In yet further embodiments, the first channel includes a first wall extending upwardly from the first side edge and a second wall extending transversely from the first wall spaced from, in overlapping relation to, the base, wherein the second channel includes a third wall extending upwardly from the second side edge and a fourth wall extending transversely from the third wall spaced from, in overlapping relation to, the base, and, wherein the second wall defines an upper surface generally coplanar to an upper surface defined by the fourth wall so as to define a common resting surface therewith.

In still further embodiments, the assembly further comprises first and second clips edge mounted to the base plate and the insert plate with the insert plate overlaying the base plate.

In some embodiments, the apertures are all similarly formed, the openings are all similarly formed, and, a first of the apertures defines an open area larger than an open area defined by a first of the openings.

In further embodiments, the apertures are each generally circular, and the openings are each generally oval shaped.

In yet further embodiments, the openings are each elongated along a longitudinal axis, a first of the openings defines a first length along the respective longitudinal axis, the first length being generally equal to a diameter of a first of the apertures.

In some embodiments, with the insert plate in the second state, the diameter of the first aperture is generally coaxial with the longitudinal axis of the first opening.

In a further aspect, provided herein is a system for preparing and/or dispensing lyospheres, the system comprising:

a first assembly including:
a base plate having a generally planar base with an array of openings formed therethrough, solid portions of the base being located between, and surrounding, the openings; and,
an insert plate for overlaying the base plate, the insert plate having a generally planar body with an array of apertures formed therethrough, the insert plate being axially shiftable relative to the base plate from a first state to a second state, wherein, the array of apertures is configured such that, in the first state, the apertures are aligned with the solid portions of the base plate with no overlap with the openings, and, in the second state, the apertures are at least partially aligned with the openings so as to at least partially overlap the openings; and,

a carrier having a bottom plate, a first upstanding side wall, and a second upstanding side wall, the carrier being configured to accommodate the first assembly above the bottom plate and between the first and second side walls,

wherein a first retention prism protrudes from the first side wall, towards the second side wall, and,

wherein the base plate is notched to shape-matingly receive the first retention prism with the first assembly being accommodated in the carrier.

In some embodiments, a second retention prism protrudes from the second side wall, towards the first side wall, and wherein the base plate is notched to shape-matingly receive the second retention prism with the first assembly being accommodated in the carrier.

In further embodiments, the insert plate is notched to shape-matingly receive the first retention prism with the first assembly being accommodated in the carrier.

In yet further embodiments, a second assembly is provided including:

a second base plate having a generally planar second base with an array of second openings formed therethrough, solid portions of the second base being located between, and surrounding, the second openings; and,

a second insert plate for overlaying the second base plate, the second insert plate having a generally planar second body with an array of second apertures formed therethrough, the second insert plate being axially shiftable relative to the second base plate from a third state to a fourth state, wherein, the array of second apertures is configured such that, in the third state, the second apertures are aligned with the solid portions of the second base plate with no overlap with the second openings, and, in the fourth state, the second apertures are at least partially aligned with the second openings so as to at least partially overlap the second openings;

wherein the first base plate includes spaced-apart first and second side edges which extend between spaced-apart first and second ends, optionally the first and second side edges each being greater in length than each of the first and second ends;

wherein the first base plate further comprises a first upstanding channel extending along the first side edge and a second upstanding channel extending along the second side edge, the first and second channels being configured to receive the first insert plate in sliding engagement to guide the first insert plate during the axial shifting of the first insert plate relative to the first base plate;

wherein the first channel includes a first wall extending upwardly from the first side edge and a second wall extending transversely from the first wall spaced from, in overlapping relation to, the first base, wherein the second channel includes a third wall extending upwardly from the second side edge and a fourth wall extending transversely from the third wall spaced from, in overlapping relation to, the first base, and, wherein the second wall defines an upper surface generally coplanar to an upper surface defined by the fourth wall so as to define a common resting surface therewith, and

wherein the second assembly is supported by the upper surfaces of the second and fourth walls of the first assembly with the first and second assemblies being accommodated in the carrier.

In some embodiments, portions of the bottom plate are raised.

In yet a further aspect, provided herein is a system for dispensing lyospheres, the system comprising:

an assembly including:

a base plate having a generally planar base with an array of openings formed therethrough, solid portions of the base being located between, and surrounding, the openings; and,

an insert plate for overlaying the base plate, the insert plate having a generally planar body with an array of apertures formed therethrough, the insert plate being axially shiftable relative to the base plate from a first state to a second state, wherein, the array of apertures is configured such that, in the first state, the apertures are aligned with the solid portions of the base plate with no overlap with the openings, and, in the second state, the apertures are at least partially aligned with the openings so as to at least partially overlap the openings; and,

a dispensing funnel having a support plate with an array of fill openings formed therethrough and a plurality of corner-shaped alignment guides protruding upwardly from the support plate around the array of fill openings, the alignment guides being configured and positioned to receive the assembly and position the assembly atop the support plate with the openings of the base plate being at least partially aligned with the fill openings of the support plate.

In some embodiments, with the assembly atop the support plate, the insert plate has a width which allows the insert plate to be axially shifted from the first state to the second state between first and second of the alignment guides.

In some embodiments, the alignment guides have inner tapered surfaces to guide the assembly to the position atop the support plate.

In certain embodiments, the first and second alignment guides define stop surfaces for interferingly engaging with portions of the base plate to inhibit movement thereof with axial shifting of the insert plate.

In other embodiments, the first and second alignment guides define secondary stop surfaces for limiting the axial shifting of the insert plate from the first state to the second state.

In some embodiments, the base plate includes first and second clips edge mounted to the base plate and the insert plate with the insert plate overlaying the base plate.

In yet other embodiments, the stop surfaces are aligned for interfering engagement with the first and second clips.

In still other embodiments, the secondary stop surfaces are aligned for interfering engagement with the base of the base plate. In yet other embodiments, a first of the fill openings extends between a first top opening, formed in a top face of the support plate, to a bottom opening, formed in a bottom face of the support plate.

In still other embodiments, the top opening has a larger area than the bottom opening.

In some embodiments, the top opening is configured to align with a plurality of the openings of the base plate with the assembly atop the support plate.

In certain embodiments, a second of the fill openings extends from a second top opening, formed in the top face of the support plate, to the first fill opening so as to merge therewith.

In other embodiments, the second fill opening merges with the first fill opening adjacent to the bottom opening.

In yet other embodiments, a first vertical axis, perpendicularly intersecting a center of the top opening, is offset from a second vertical axis, perpendicularly intersecting a center of the bottom opening.

In still other embodiments, the dispensing funnel further includes a removable collection plate intersecting the fill openings.

### BRIEF DESCRIPTION OF THE DRAWINGS

FIG. 1 shows temperature kinetics of a shelf inlet and assembly levels 2-9 in an aluminum stack.

FIGS. 2A-2D illustrate different configurations of an assembly stack. In FIG. 2A, an 8-level stack with frozen droplets on each level is placed directly on a shelf of a lyophilizer, and level 1 base plate that is in full contact with the shelf has frozen droplets on it. In FIG. 2B, a 9-level stack with frozen droplets on levels 2-9 is placed on a shelf of a lyophilizer, and level 1 base plate that is in full contact with the shelf has no frozen droplets on it. In FIG. 2C, an 8-level stack with frozen droplets on each level is placed on thermal conduction side spacers on a shelf of a lyophilizer, so that level 1 base plate that has frozen droplets on it is not in full contact with the shelf. In FIG. 2D, a thermally conductive rack with multiple levels is placed between two shelves of a lyophilizer. One assembly can be placed on each level of the rack. The broken lines in the middle of each level indicate that each level of the rack can be a full board or two portions at the ends.

FIGS. 3A and 3B demonstrate the surprising discovery that the temperature of the bottom base plate that is in full contact with the lyophilizer shelf departed significantly from that of the other base plates in the same stack. FIG. 3A shows a prominent dip in the thermocouple trace for level 1 that is in full contact with the lyophilizer shelf. FIG. 3B shows that the temperatures of levels 2-9, which are not in full contact with the lyophilizer shelf, tracked each other well and are much more consistent with each other over time.

FIGS. 4A and 4B compare temperature kinetics and uniformity among assembly levels between a stack of assemblies comprising stainless steel base plates and stainless steel thermally conductive spacers and a stack of assemblies comprising aluminum base plates and aluminum thermally conductive spacers. FIG. 4A shows temperature kinetics of levels 2-9 in a stainless-steel stack with frozen droplets. FIG. 4B shows temperature kinetics of levels 2-9 in an aluminum stack.

FIGS. 5 and 6 show an assembly useable with the subject invention. FIG. 5 shows the assembly in a first state. FIG. 6 shows the assembly in a second state.

FIGS. 7 and 8 show a base plate useable with the subject invention. FIG. 8 is a cross-sectional view taken along line 8-8 of FIG. 7.

FIG. 9 shows an insert plate useable with the subject invention.

FIG. 10 shows an array of openings useable with the base plate in accordance with the subject invention.

FIG. 11 shows an array of apertures useable with the insert plate in accordance with the subject invention.

FIG. 12 shows the assembly in the first state.

FIG. 13 shows the assembly in the second state.

FIG. 14 shows alternate openings and apertures useable with the subject invention.

FIG. 15 shows a carrier useable with the subject invention.

FIGS. 16-18 show a carrier accommodating assemblies therein in accordance with the subject invention. FIG. 16 shows the carrier accommodating a stack of the assemblies. FIGS. 17 and 18 show a carrier accommodating one assembly.

FIG. 19 shows lyospheres accommodated by an assembly in the first state.

FIGS. 20-23 show a dispensing funnel accommodating an assembly in accordance with the subject invention. FIG. 20 shows the assembly accommodated in the dispensing funnel. FIG. 21 shows an alignment guide useable with the dispensing funnel. FIG. 22 shows the assembly accommodated in the dispensing funnel in the first state. FIG. 23 shows the assembly accommodated in the dispensing funnel in the second state.

FIG. 24 shows an alternative base tray having closed wells.

FIGS. 25A-25D demonstrate that an assembly or a stack of assemblies are placed on top of a heat sink without being physically attached to the heat sink. FIG. 25A shows a heat sink. FIG. 25B shows an assembly placed on top of the heat sink without being physically attached to the heat sink. FIG. 25C shows the assembly with an array of frozen droplets on the base plate while sitting on top of the heat sink. FIG. 25D shows a stack of assemblies are placed on top of the heat sink. The assembly and the stack can be easily moved away from the heat sink.

FIG. 26 shows thermocouple temperatures during lyophilization, the shelf inlet temperature, and TDLAS data showing mass of removed water, all as a function of time in Example 5.

FIG. 27 shows the water moisture level in the headspace of each vial in parts per million (ppm), with the vial positions shown as they were in the array when they were filled with lyospheres in Example 5.

FIG. 28 shows thermocouple temperatures during lyophilization, the shelf inlet temperature, and TDLAS data showing mass of removed water, all as a function of time in Example 6.

FIGS. 29A-29D show lyobeads dispensed by four different methods, illustrating ways to use a combiner dispensing funnel or an accumulator dispensing funnel. FIG. 29A shows one assembly of 100 white lyobeads dispensed into 50 vials using a combiner dispensing funnel. FIG. 29B shows 3 assemblies of 100 white lyobeads dispensed, one after another, into 50 vials using a combiner dispensing funnel. FIG. 29C shows 2 assemblies of 100 lyobeads (one assembly with alternate rows of white and red lyospheres, the other assembly with all red lyospheres) dispensed, one after another, into 50 vials using a combiner dispensing funnel. FIG. 29D shows 2 assemblies of 100 lyobeads (one assembly with all white lyospheres, the other assembly with all red lyospheres) dispensed into an accumulator dispensing funnel then 100 vials.

FIG. 30 shows thermocouple temperatures during lyophilization, the shelf inlet temperature, and TDLAS data showing mass of removed water, all as a function of time in Example 7.

FIG. 31 shows thermocouple temperatures during lyophilization, the shelf inlet temperature, and TDLAS data showing mass of removed water, all as a function of time in Example 8.

FIGS. 32A and 32B show good visual appearance of lyospheres of a pharmaceutical composition comprising a vaccine formulation and an adjuvant formulation that were mixed immediately before dispensing and freezing liquid droplets. FIG. 32A shows an array of 100 lyobead-filled syringes, and FIG. 32B shows a closeup photograph of 3 of the lyobead-filled syringes.

FIGS. 33A-33D illustrate a dispensing funnel with a plurality of fill openings combined to dispense a plurality of lyospheres into individual target containers. FIG. 33A is a cross-sectional view of a dispensing funnel with multiple fill openings being combined. FIG. 33B is a bottom plan view of a dispensing funnel with multiple fill openings being combined. FIGS. 33C-33D are views of a plurality of fill openings being combined.

FIGS. 34A-34D illustrate a dispensing funnel with a slot for receiving a removable collection plate. FIGS. 34A-34B show the dispensing funnel with the slot being open to receive a collection plate. FIG. 34C is a cross-sectional view showing a removable collection plate being received in a slot of a dispensing funnel. FIG. 34D is a photograph of a dispensing funnel with a slot for receiving a removable collection plate.

FIGS. 35A-35H illustrate a dispensing funnel with fill openings having offset top and bottom openings. FIGS. 35A-35C are different views of a dispensing funnel with fill openings having offset top and bottom openings. FIG. 35D is a cross-sectional view of a dispensing funnel with fill openings having offset top and bottom openings. FIGS. 35E-35H show fill openings with varied offsets between top and bottom openings.

FIGS. 36A-36F show an assembly useable with the subject invention where channels are provided as separate components from the base plate. FIG. 36A shows the assembly before assembly of the base plate and the insert plate. FIGS. 36B-36F are different views of an assembly with channels being provided as separate components.

FIGS. 37A-37I show a dispensing funnel with various configurations of stop surfaces for inhibiting movement of components of an assembly during dispensing of lyospheres.

FIGS. 38A-38D show a dispensing funnel with top openings of fill openings being configured to each align with a plurality of openings of a base plate to allow a plurality of lyospheres to be dispensed into individual target containers.

### DETAILED DESCRIPTION OF THE INVENTION

### Definitions

Unless described otherwise, all technical and scientific terms used herein have the same meaning as is commonly understood by one of ordinary skill in the art. For purpose of interpreting this specification, the following description of terms will apply and whenever appropriate, terms used in the singular will also include the plural and vice versa. All patents, applications, published applications, and other publications are incorporated herein by reference in their entirety. In the event that any description of terms set forth conflicts with any document incorporated herein by reference, the description of term set forth below shall control.

The term “lyosphere” or “lyobead” as used herein, refers to a droplet of a liquid material that is frozen and dried. The liquid material can be any material that is in the liquid state or suspended in liquid, including but not limited to solutions, suspensions, emulsions, foams, sols, gels, semisolids, melts, or mixtures thereof. In some embodiments, the liquid material is a pharmaceutical composition.

“Array” or “array format,” as used herein, refers to any arrangement of locations, including but not limited to parallel rows (in-phase or staggered), parallel arcs (in-phase or staggered), and/or irregular patterns. In the embodiment of parallel rows, the locations can be arranged in rows and columns. In certain embodiments, the rows and/or columns align with each other (i.e., in-phase). In other embodiments, the rows and/or columns are staggered relative to each other. In the embodiment of parallel arcs, the locations are arranged in circles or arcs around a common point. In certain embodiments, the circles or arcs align with each other (i.e., in-phase). In other embodiments, the circles or arcs are staggered relative to each other. In certain embodiments, the locations can be arranged with a regular repeating pattern throughout the entire array. In certain embodiments, the locations can be arranged in a combination of different patterns within an array.

The term “about” or “approximately” means within 20%, within 15%, within 10%, within 9%, within 8%, within 7%, within 6%, within 5%, within 4%, within 3%, within 2%, within 1%, or less of a given value or range.

A “heat sink,” as used herein, refers to a device or substance for absorbing heat. In some embodiments, the heat sink is a passive heat exchanger that transfers the heat away from a source to a fluid medium, such as air or a liquid coolant (e.g., liquid nitrogen).

The term “not physically attached to,” when used in the context of a base plate and a heat sink, means that the base plate is not physically fixed to the heat sink by any approaches, including but not limited to screws, straps, pressure clamps or clips, welding, gluing with a cryoglue, etc., and that the base plate and the heat sink are not fabricated together as a single device from a single metal block. When the base plate is “not physically attached to” the heat sink, the base plate can be chilled without requiring connection or attachment to the heat sink and can be easily transferred away from the heat sink without requiring disconnection or unattachment.

“A thermally conductive path,” as used herein, means that a path within a thermally conductive material or through multiple thermally conductive components that are in physical contact, along which heat can transfer from one location to another. A “thermally conductive” material or component means that the material or component has a thermal conductivity of at least 1 Watt/meter*kelvin (W/m*k). In various embodiments, a thermally conductive material or component has a thermal conductivity of at least 2, 3, 4, 5, 6, 7, 8, 9, or 10 W/m*k. The thermally conductive component can be anything thermally conductive, such as a thermally conductive spacer, connector, wire, block, rack, level, shelf, or board, etc.

“Full contact,” when used in the context of two generally planar surfaces, means that at least 50% of the smaller of the two surfaces are in physical contact. In various embodiments, a full contact between two generally planar surfaces means that at least 60%, 70%, 80%, 90%, 95%, 96%, 97%, 98%, or 99% of the smaller of the two surfaces are in physical contact. When two generally planar surfaces are “not in full contact,” less than 50% of the smaller of the two surfaces are in physical contact. In various embodiments, being not in full contact between two generally planar surfaces means that less than 40%, 30%, 20%, 10%, 5%, 4%, 3%, 2%, or 1% of the smaller of the two surfaces are in physical contact.

The term “clip” as used herein refers to a metal device capable of holding more than one object (e.g., a base plate and an insert plate) together. A clip is a type of fastener and has multiple surfaces between which objects can be positioned and held, fastened, or clipped together. Clips can take many different shapes to advantageously hold pluralities of the same or different objects together. A clip can exert at least minimal pressure to hold objects between surfaces of the clip together; however, the clamping or holding force of the clip may be small enough to allow one object within the clip (e.g., an insert plate) to be moved or repositioned relative to another object within the clip (e.g., a based plate) with only modest effort, or to be removed from the collection of objects being clipped together. The clamping force of clips can span a very wide range.

A “clip-style assembly” refers to an assembly that comprises a base plate, an insert plate, and two clips edge mounted to the base plate and the insert plate with the insert plate overlaying the base plate.

### Methods for Preparing and/or Dispensing Lyospheres of Pharmaceutical Compositions

Production of lyospheres usually involves dispensing droplets of liquid pharmaceutical compositions, freezing the droplets, lyophilizing the frozen droplets to produce lyospheres, storage of lyospheres, and dispensing lyospheres into final containers. One lab-scale process is to dispense droplets (using an automated high throughput liquid handler) onto the top of a chilled surface that is integral to or attached to a heat sink, freeze the droplets on the cold surface, scrape the frozen droplets off the cold surface into a cold collection bin, transfer the frozen droplets into a cold tray accumulating at a depth of from 1 to 5 irregularly packed layers of frozen droplets, lyophilize the frozen droplets to produce lyospheres, collect lyospheres in a glass bottle for storage, and dispense lyospheres into vials either manually or using a dispensing machine that vibrates lyospheres to move them through a series of chutes and rails to singulate lyospheres and dispenses one lyosphere at a time into one vial at a time.

This process can have multiple problems, including lyosphere breakage, dusting, static electricity associated with vibrating and handling the lyospheres, low dispense throughput, dispense errors (e.g., more than one lyosphere is dispensed at a time into one vial), process sensitivity to lyosphere size and shape, and the lack of an ensured first-lyosphere-in, first-lyosphere-out product flow.

Thus, methods for dispensing and freezing droplets of pharmaceutical compositions in an array format, drying the frozen droplets in the same array format (without dislocating them from the surface on which they are frozen) in single layers, and dispensing lyospheres in the array format into containers in the same array format are developed to improve the lyosphere production and dispensing process. These methods in the array format can be achieved by using an assembly comprising a base plate. In some embodiments, these methods in the array format are achieved by using an assembly comprising a base plate and an insert plate overlaying the base plate. In certain embodiments, the insert plate has an array of apertures. In other embodiments, the base plate has an array of openings with solid portions between and surrounding the openings. In some other embodiments, the insert plate has an array of apertures, and the base plate has an array of openings with solid portions between and surrounding the openings.

One benefit of the methods described herein is that an assembly holds droplets of pharmaceutical compositions through the freezing, lyophilization, storage, and dispensing steps of the entire process. A single contact holder for pharmaceutical compositions from liquid dispense all the way through lyosphere dispense eliminates the need for transfer between individual unit operations of freezing, drying, and dispensing into vials. This has significant benefits in several aspects, including avoiding static electricity buildup, minimizing lyosphere damage or loss, and eliminating the step of singulating lyospheres before a counting and dispensing operation.

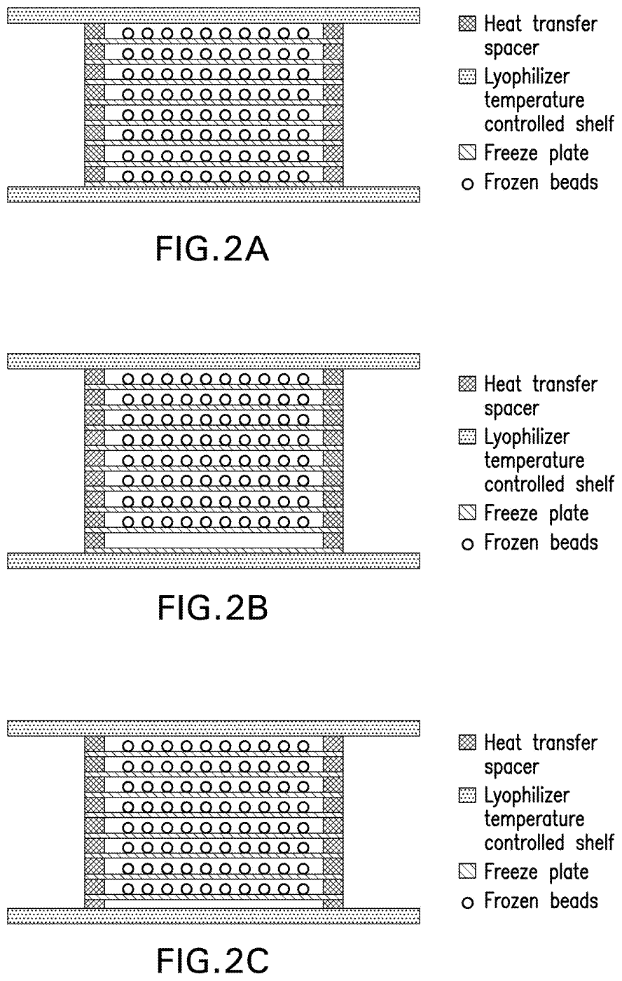

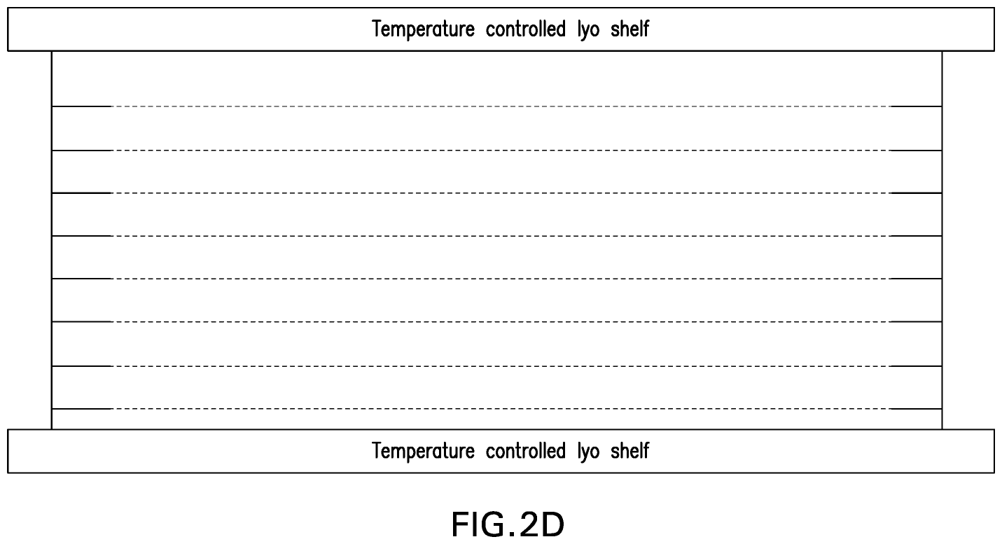

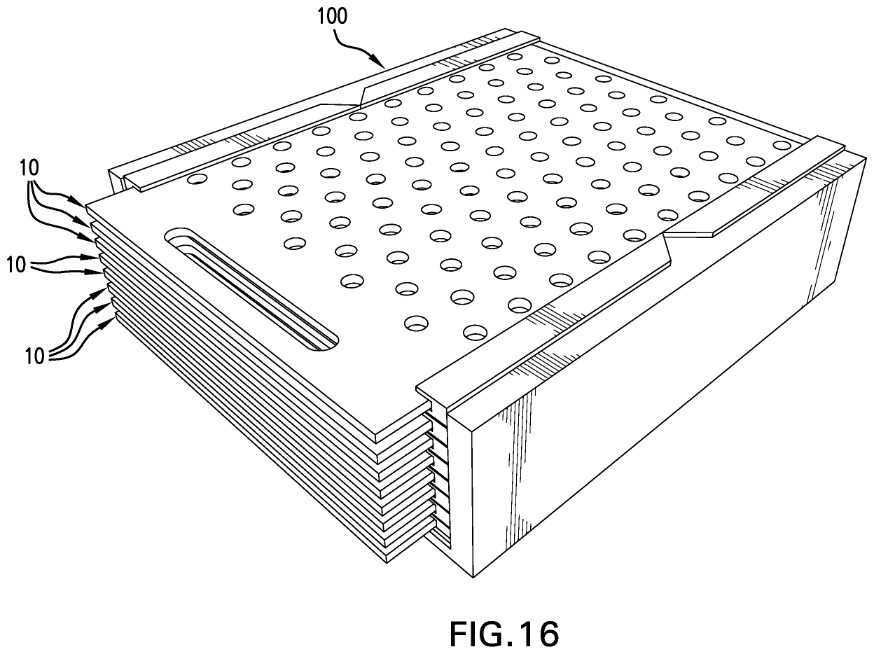

Another benefit is that frozen droplets can be lyophilized in monolayers in assembly stacks. When frozen droplets are lyophilized as a monolayer, for example, in direct contact with a metal surface, conductive heat transfer between the frozen droplets and the metal surface is high. When frozen droplets are collected in trays in a bulk quantity that exceeds the monolayer capacity of the tray, the frozen droplets can stack upon each other to a depth of up to 5 layers. In that case, while the bottom frozen droplets in the tray are in direct contact with the tray, the top ones are not. For the frozen droplets that are not in direct contact with the metal tray, conductive heat transfer through other frozen droplets is less efficient than heat transfer through direct contact with a thermally conductive tray, shelf, or base plate. Multilayer stacking of frozen droplets upon each other leads to longer lyophilization cycle times than monolayers and can contribute to variability of properties (such as moisture content) from layer to layer within the tray. In contrast, when using the various assemblies described herein, the assemblies, each containing a monolayer of frozen droplet, can be stacked vertically forming a thermally conductive path between the base plates (e.g., through thermally conductive spacers (FIG. 2C), thermally conductive racks (FIG. 2D), or thermally conductive raised edges of the base plates (FIG. 16), etc.) for efficient heat transfer. In these stacks of assemblies with monolayers of frozen droplets, each frozen droplet sits directly on a thermally conductive base plate surface. Using the shelf moving (stoppering) capability of a lyophilizer, the stacks of assemblies can be sandwiched between two lyophilizer shelves, resulting in conductive heat transfer from both below and above the stack, with efficient heat distribution and equilibration throughout the entire stack, thereby rendering more uniform drying and product quality attributes. A stacked arrangement of assemblies multiplies drying throughput without compromising drying uniformity. Thus, the drying time and product quality advantages of monolayer drying are combined with the capacity and throughput advantages of drying stacks of assemblies.

A third benefit is that the heat transfer benefits described above and maintaining lyospheres separate from each other during lyophilization enable the annealing of frozen droplets within the lyophilizer prior to drying. Annealing is a method of raising the frozen product temperature above its glass transition temperature to re-structure the ice crystals or crystallize certain other excipients. This process is used in traditional vial drying processes to reduce cycle time, improve uniformity, and/or improve stability. However, drying of frozen droplets in bulk tray format is often not compatible with annealing because the annealing process can cause frozen droplets that are in physical contact with each other to stick together. Therefore, the methods, devices, systems, and approaches described in this invention would enable annealing because frozen droplets are not in contact with each other. Furthermore, the tight and rapid temperature control afforded by this method would allow control of product temperature above the glass transition without melting the product.

A fourth benefit is to improve the efficiency and quality of lyosphere dispense. When lyospheres are poured into storage containers after lyophilization and again poured onto a vibrating hopper at the time of lyosphere dispense, the pouring operations and movement of lyospheres against each other and different surfaces can lead to buildup of static electricity, which can cause difficulties during handling and dispense. Without special care and planning, storage in a bulk format can damage lyospheres, as can vibration on metal plates and rails employed during piezoelectric dispensing. It is also possible that the first lyospheres added to the piezoelectric dispenser might not be the first lyospheres dispensed into vials, leaving some lyospheres to vibrate within the hopper for a long time, resulting in dusting and variability among lyospheres. In one lyosphere dispensing process, lyospheres are vibrated along rails until they form a single file line of singulated lyospheres and then are dropped one at a time into one vial at a time. This dispense process is slow and error prone. For example, double dispenses can occur when two lyospheres are not properly singulated and fall into one vial at the same time. Double dispense has been challenging to eliminate and in some cases even hard to detect. By using the capability of the liquid dispensing equipment (early in the process at the step of freezing) to position droplets during the freezing step in precise positions that match the positions of containers in container nests, and using an assembly to maintain the droplets in those positions through the freezing, lyophilization, and storage steps, the produced lyospheres arrive (later in the process at the step of lyosphere dispense) pre-positioned optimally for dispense into vials, pre-Tillable syringes, cartridges, or other containers in nests. A funnel support plate securely directs the lyospheres from the assembly into nested vials, syringes, cartridges, or other containers to achieve accurate and fast dispense of the already singulated lyospheres.

The methods described herein are compatible with high throughput automation and quality detection systems. Compared with the vibratory dispense methods, the methods described herein have the benefit of reducing variation in the time that lyospheres are exposed to vibratory mechanical stress. Assembly stacks can remain sealed in moisture barrier bags until ready for dispense, minimizing lyospheres' exposure time to moisture in the dispensing chamber atmosphere.

The methods described herein can be implemented and adapted for use in drying equipment beyond typical lyophilizers. These include lyophilizers with different features such as temperature-controlled walls, and/or temperature-controlled shelves in horizontal, vertical, or mixed configurations. One skilled in the art will recognize other methods and equipment to impart thermal energy to dry lyospheres within the scope of the methods, devices, systems and approaches of this disclosure.

In summary, benefits of the described methods in the lyosphere production and dispensing process include but are not limited to: (1) reduced sensitivity to bead differences in formulations, sizes, strengths, friabilities, or shapes, which is particularly valuable for GMP clinical trial production equipment, for which flexibility is important to process different products; (2) allows convenient and efficient automation methods (including robotics) to be applied to the process, in which assemblies and stacks rather than individual frozen droplets or lyospheres are moved; (3) well suited for annealing of frozen droplets before lyophilization; (4) increased speed and reduced error rate of lyosphere dispense into final containers; (5) well suited to inspection systems including visual inspection systems of lyospheres in array formats; (6) well suited for dispensing combination products, which can be achieved by co-packaging lyospheres of different pharmaceutical compositions in a single container; (7) reduced risks for bead breakage and buildup of static electricity; (8) easily adaptable to different containers and container sizes, taking advantage of vial, cartridges, and pre-fillable syringe nest and tub technology that has been developed; and (9) shorter drying cycles and improved product uniformity during lyophilization (comparing to the conventional approach of drying multilayers of frozen droplets in trays) while maintaining (or even exceeding) lyophilization cabinet drying throughput.

In one aspect, provided herein is a method for freezing droplets of a pharmaceutical composition, comprising:
(a) providing an assembly comprising a generally planar base plate, wherein the base plate is placed on top of a heat sink and chilled to a low temperature, wherein the base plate is not physically attached to the heat sink; and
(b) dispensing droplets of the pharmaceutical composition on the base plate in an array format, wherein the droplets freeze on the base plate.

In certain embodiments, the assembly further comprises an insert plate overlaying the base plate; wherein the insert plate has an array of apertures; and the droplets of the pharmaceutical composition are dispensed into the apertures to be supported by the base plate.

Thus, in certain embodiments, the method for freezing droplets of a pharmaceutical composition comprises:
(a) providing an assembly comprising an insert plate overlaying a generally planar base plate; wherein the base plate is placed on top of a heat sink and chilled to a low temperature; wherein the base plate is not physically attached to the heat sink; wherein the insert plate has an array of apertures; and
(b) dispensing droplets of the pharmaceutical composition into the apertures to be supported by the base plate, wherein the droplets freeze on the base plate.

In other embodiments, the assembly further comprises an insert plate overlaying the base plate; wherein the insert plate has an array of apertures; wherein the base plate has an array of openings and solid portions located between and surrounding the openings; wherein the apertures align with the solid portions of the base plate with no overlap with the openings; and the droplets of the pharmaceutical composition are dispensed into the apertures to be supported by the solid portions of the base plate.

Thus, in other embodiments, the method for freezing droplets of a pharmaceutical composition comprises:
(a) providing an assembly comprising an insert plate overlaying a generally planar base plate; wherein the base plate is placed on top of a heat sink and chilled to a low temperature; wherein the base plate is not physically attached to the heat sink; wherein the insert plate has an array of apertures; wherein the base plate has an array of openings and solid portions located between and surrounding the openings; wherein the apertures align with the solid portions of the base plate with no overlap with the openings; and
(b) dispensing droplets of the pharmaceutical composition into the apertures to be supported by the solid portions of the base plate, wherein the droplets freeze on the base plate.

In another aspect, provided herein is a method for preparing lyospheres of a pharmaceutical composition, comprising:
(a) providing an assembly comprising a generally planar base plate, wherein the base plate is placed on top of a heat sink and chilled to a low temperature, wherein the base plate is not physically attached to the heat sink;
(b) dispensing droplets of the pharmaceutical composition on the base plate in an array format, wherein the droplets freeze on the base plate; and
(c) placing the assembly in a lyophilizer to dry the frozen droplets and produce an array of lyospheres.

In some embodiments, the method of preparing lyospheres of a pharmaceutical composition comprises repeating steps (a) and (b) multiple times, preparing a stack of assemblies with a thermally conductive path formed between the assemblies, and in step (c) drying the frozen droplets in the entire stack in a lyophilizer to produce arrays of lyospheres.

In one embodiment, the thermally conductive path is formed by stacking a plurality of assemblies on top of each other, wherein each base plate has at least two raised edges, and wherein the raised edges of the base plates are in physical contact. In some embodiments, the stack of assemblies can be prepared outside of the lyophilizer. In other embodiments, the stack of assemblies can be prepared inside of the lyophilizer. In certain embodiments of this method, in step (c) the lowest base plate is not in full contact with a shelf of the lyophilizer.

In another embodiment, the thermally conductive path is formed by placing thermally conductive spacers along at least two edges of each base plate and stacking a plurality of assemblies on top of each other, wherein the spacers and the base plates are in physical contact. In some embodiments, the stack of assemblies can be prepared outside of the lyophilizer. In other embodiments, the stack of assemblies can be prepared inside of the lyophilizer. In certain embodiments of this method, in step (c) the lowest base plate is not in full contact with a shelf of the lyophilizer.

In yet another embodiment, the thermally conductive path is formed by providing a thermally conductive rack with multiple levels and placing a plurality of assemblies on the levels of the rack, wherein the base plates and the levels of the rack are in physical contact. In some embodiments, the stack of assemblies can be prepared outside of the lyophilizer. In other embodiments, the stack of assemblies can be prepared inside of the lyophilizer. In certain embodiments of this method, in step (c) the lowest base plate is not in full contact with a shelf of the lyophilizer.

In still another embodiment, the thermally conductive path is formed by edge mounting two thermally conductive clips to the base plate and the insert plate with the insert plate overlaying the base plate and stacking a plurality of assemblies on top of each other, wherein the clips are in physical contact. In certain embodiments of this method, in step (c) the lowest base plate is not in full contact with a shelf of the lyophilizer.

In still another embodiment, the thermally conductive path is formed by combining two, three, or four of the above described methods. In some embodiments, the stack of assemblies can be prepared outside of the lyophilizer. In other embodiments, the stack of assemblies can be prepared inside of the lyophilizer. In certain embodiments of this method, in step (c) the lowest base plate is not in full contact with a shelf of the lyophilizer.

In certain embodiments, the assembly further comprises an insert plate overlaying the base plate; wherein the insert plate has an array of apertures; and wherein each droplet is dispensed into an aperture to be supported by the base plate.

In other embodiments, the assembly further comprises an insert plate overlaying the base plate; wherein the insert plate has an array of apertures; wherein the base plate has an array of openings and solid portions located between and surrounding the openings; wherein the apertures align with the solid portions of the base plate with no overlap with the openings; and wherein each droplet is dispensed into an aperture to be supported by the solid portions of the base plate.

Thus, in some embodiments, the method for preparing lyospheres of a pharmaceutical composition comprises:
(a) providing an assembly comprising an insert plate overlaying a generally planar base plate; wherein the base plate is placed on top of a heat sink and chilled to a low temperature; wherein the base plate is not physically attached to the heat sink; wherein the insert plate has an array of apertures;
(b) dispensing droplets of the pharmaceutical composition into the apertures to be supported by the base plate, wherein the droplets freeze on the base plate; and
(c) placing the assembly in a lyophilizer to dry the frozen droplets and produce an array of lyospheres.

In other embodiments, the method for preparing lyospheres of a pharmaceutical composition comprises:
(a) providing an assembly comprising an insert plate overlaying a generally planar base plate; wherein the base plate is placed on top of a heat sink and chilled to a low temperature; wherein the base plate is not physically attached to the heat sink; wherein the insert plate has an array of apertures; wherein the base plate has an array of openings and solid portions located between and surrounding the openings; wherein the apertures align with the solid portions of the base plate with no overlap with the openings;
(b) dispensing droplets of the pharmaceutical composition into the apertures to be supported by the solid portions of the base plate, wherein the droplets freeze on the base plate; and
(c) placing the assembly in a lyophilizer to dry the frozen droplets and produce an array of lyospheres.

In yet other embodiments, the method for preparing lyospheres of a pharmaceutical composition comprises:
(a) providing an assembly comprising an insert plate overlaying a generally planar base plate; wherein the base plate is placed on top of a heat sink and chilled to a low temperature; wherein the base plate is not physically attached to the heat sink; wherein the insert plate has an array of apertures;
(b) dispensing droplets of the pharmaceutical composition into the apertures to be supported by the base plate, wherein the droplets freeze on the base plate; repeating steps (a) and (b) multiple times and preparing a stack of assemblies with a thermally conductive path formed between the assemblies; and
(c) drying the frozen droplets in the entire stack in a lyophilizer to produce arrays of lyospheres.

In still other embodiments, the method for preparing lyospheres of a pharmaceutical composition comprises:
(a) providing an assembly comprising an insert plate overlaying a generally planar base plate; wherein the base plate is placed on top of a heat sink and chilled to a low temperature; wherein the base plate is not physically attached to the heat sink; wherein the insert plate has an array of apertures; wherein the base plate has an array of openings and solid portions located between and surrounding the openings; wherein the apertures align with the solid portions of the base plate with no overlap with the openings;
(b) dispensing droplets of the pharmaceutical composition into the apertures to be supported by the solid portions of the base plate, wherein the droplets freeze on the base plate; repeating steps (a) and (b) multiple times and preparing a stack of assemblies with a thermally conductive path formed between the assemblies; and
(c) drying the frozen droplets in the entire stack in a lyophilizer to produce arrays of lyospheres.

In one embodiment, the thermally conductive path is formed by stacking a plurality of assemblies on top of each other, wherein each base plate has at least two raised edges, and wherein the raised edges of the base plates are in physical contact. In some embodiments, the stack of assemblies can be prepared outside of the lyophilizer. In other embodiments, the stack of assemblies can be prepared inside of the lyophilizer. In certain embodiments of this method, in step (c) the lowest base plate is not in full contact with a shelf of the lyophilizer.

In another embodiment, the thermally conductive path is formed by placing thermally conductive spacers along at least two edges of each base plate and stacking a plurality of assemblies on top of each other, wherein the spacers and the base plates are in physical contact. In some embodiments, the stack of assemblies can be prepared outside of the lyophilizer. In other embodiments, the stack of assemblies can be prepared inside of the lyophilizer. In certain embodiments of this method, in step (c) the lowest base plate is not in full contact with a shelf of the lyophilizer.

In yet another embodiment, the thermally conductive path is formed by providing a thermally conductive rack with multiple levels and placing a plurality of assemblies on the levels of the rack, wherein the base plates and the levels of the rack are in physical contact. In some embodiments, the stack of assemblies can be prepared outside of the lyophilizer. In other embodiments, the stack of assemblies can be prepared inside of the lyophilizer. In certain embodiments of this method, in step (c) the lowest base plate is not in full contact with a shelf of the lyophilizer.

In still another embodiment, the thermally conductive path is formed by edge mounting two thermally conductive clips to the base plate and the insert plate with the insert plate overlaying the base plate and stacking a plurality of assemblies on top of each other, wherein the clips are in physical contact. In certain embodiments of this method, in step (c) the lowest base plate is not in full contact with a shelf of the lyophilizer.

In still another embodiment, the thermally conductive path is formed by combining two, three, or four of the above described methods. In some embodiments, the stack of assemblies can be prepared outside of the lyophilizer. In other embodiments, the stack of assemblies can be prepared inside of the lyophilizer. In certain embodiments of this method, in step (c) the lowest base plate is not in full contact with a shelf of the lyophilizer.

In some embodiments, the method for preparing lyospheres of a pharmaceutical composition comprises:
(a) providing an assembly comprising an insert plate overlaying a generally planar base plate; wherein the base plate is placed on top of a heat sink and chilled to a low temperature; wherein the base plate is not physically attached to the heat sink; wherein the insert plate has an array of apertures;
(b) dispensing droplets of the pharmaceutical composition into the apertures to be supported by the base plate, wherein the droplets freeze on the base plate; and
(c) placing the assembly in a lyophilizer to dry the frozen droplets and produce an array of lyospheres; wherein the base plate is not in full contact with a shelf of the lyophilizer.

In other embodiments, the method for preparing lyospheres of a pharmaceutical composition comprises:
(a) providing an assembly comprising an insert plate overlaying a generally planar base plate; wherein the base plate is placed on top of a heat sink and chilled to a low temperature; wherein the base plate is not physically attached to the heat sink; wherein the insert plate has an array of apertures; wherein the base plate has an array of openings and solid portions located between and surrounding the openings; wherein the apertures align with the solid portions of the base plate with no overlap with the openings;
(b) dispensing droplets of the pharmaceutical composition into the apertures to be supported by the solid portions of the base plate, wherein the droplets freeze on the base plate; and
(c) placing the assembly in a lyophilizer to dry the frozen droplets and produce an array of lyospheres; wherein the base plate is not in full contact with a shelf of the lyophilizer.

In yet other embodiments, the method for preparing lyospheres of a pharmaceutical composition comprises:
(a) providing an assembly comprising an insert plate overlaying a generally planar base plate; wherein the base plate is placed on top of a heat sink and chilled to a low temperature; wherein the base plate is not physically attached to the heat sink; wherein the insert plate has an array of apertures; wherein the base plate has at least two raised edges so that a plurality of assemblies can be stacked on top of each other with the raised edges of the base plates in physical contact;
(b) dispensing droplets of the pharmaceutical composition into the apertures to be supported by the base plate, wherein the droplets freeze on the base plate; repeating steps (a) and (b) multiple times and stacking the assemblies on top of each other to prepare a stack of assemblies with frozen droplets; and
(c) drying the frozen droplets in the entire stack in a lyophilizer to produce arrays of lyospheres; wherein the base plate is not in full contact with a shelf of the lyophilizer.

In still other embodiments, the method for preparing lyospheres of a pharmaceutical composition comprises:
(a) providing an assembly comprising an insert plate overlaying a generally planar base plate; wherein the base plate is placed on top of a heat sink and chilled to a low temperature; wherein the base plate is not physically attached to the heat sink; wherein the insert plate has an array of apertures; wherein the base plate has an array of openings and solid portions located between and surrounding the openings; wherein the apertures align with the solid portions of the base plate with no overlap with the openings; wherein the base plate has at least two raised edges so that a plurality of assemblies can be stacked on top of each other with the raised edges of the base plates in physical contact;
(b) dispensing droplets of the pharmaceutical composition into the apertures to be supported by the solid portions of the base plate, wherein the droplets freeze on the base plate; repeating steps (a) and (b) multiple times and stacking the assemblies on top of each other to prepare a stack of assemblies with frozen droplets; and
(c) drying the frozen droplets in the entire stack in a lyophilizer to produce arrays of lyospheres; wherein the base plate is not in full contact with a shelf of the lyophilizer.

In yet another aspect, provided herein is a method of preparing lyospheres of a pharmaceutical composition, comprising:
(a) providing an assembly comprising an insert plate overlaying a base plate; wherein the base plate has an array of openings and solid portions located between and surrounding the openings; wherein the insert plate has an array of apertures; wherein the insert plate is axially shiftable relative to the base plate from a first state to a second state; wherein in the first state, the apertures align with the solid portions of the base plate with no overlap with the openings, and in the second state, the apertures at least partially align with the openings so as to at least partially overlap with the openings; wherein the base plate is chilled to a low temperature;
(b) while the insert plate is in the first state, dispensing droplets of the pharmaceutical composition into the apertures to be supported by the solid portions of the base plate, wherein the droplets freeze on the base plate;
(c) placing the assembly in a lyophilizer to dry the frozen droplets and produce lyospheres;
(d) providing a container nest comprising an array of pharmaceutically acceptable containers, each of the containers having a loading opening, wherein the openings in the base plate align with the loading openings of the containers; and
(e) dispensing the lyospheres into the containers by axially shifting the insert plate relative to the base plate to the second state.

In some embodiments, the method of preparing lyospheres of a pharmaceutical composition comprises repeating steps (a)-(b) multiple times, preparing a stack of assemblies with a thermally conductive path formed between the assemblies; in step (c) drying the frozen droplets in the entire stack in a lyophilizer to produce lyospheres; and repeating steps (d)-(e) multiple times to dispense the lyospheres in each assembly into the containers in a container nest.

In one embodiment, the thermally conductive path is formed by stacking a plurality of assemblies on top of each other, wherein each base plate has at least two raised edges, and wherein the raised edges of the base plates are in physical contact. In some embodiments, the stack of assemblies can be prepared outside of the lyophilizer. In other embodiments, the stack of assemblies can be prepared inside of the lyophilizer. In certain embodiments of this method, in step (c) the lowest base plate is not in full contact with a shelf of the lyophilizer.

In another embodiment, the thermally conductive path is formed by placing thermally conductive spacers along at least two edges of each base plate and stacking a plurality of assemblies on top of each other, wherein the spacers and the base plates are in physical contact.

In some embodiments, the stack of assemblies can be prepared outside of the lyophilizer. In other embodiments, the stack of assemblies can be prepared inside of the lyophilizer. In certain embodiments of this method, in step (c) the lowest base plate is not in full contact with a shelf of the lyophilizer.

In yet another embodiment, the thermally conductive path is formed by providing a thermally conductive rack with multiple levels and placing a plurality of assemblies on the levels of the rack, wherein the base plates and the levels of the rack are in physical contact. In some embodiments, the stack of assemblies can be prepared outside of the lyophilizer. In other embodiments, the stack of assemblies can be prepared inside of the lyophilizer. In certain embodiments of this method, in step (c) the lowest base plate is not in full contact with a shelf of the lyophilizer.

In still another embodiment, the thermally conductive path is formed by edge mounting two thermally conductive clips to the base plate and the insert plate with the insert plate overlaying the base plate and stacking a plurality of assemblies on top of each other, wherein the clips are in physical contact. In certain embodiments of this method, in step (c) the lowest base plate is not in full contact with a shelf of the lyophilizer.

In still another embodiment, the thermally conductive path is formed by combining two, three, or four of the above described methods. In some embodiments, the stack of assemblies can be prepared outside of the lyophilizer. In other embodiments, the stack of assemblies can be prepared inside of the lyophilizer. In certain embodiments of this method, in step (c) the lowest base plate is not in full contact with a shelf of the lyophilizer.

Thus, in some embodiments, the method of preparing lyospheres of a pharmaceutical composition comprises:
(a) providing an assembly comprising an insert plate overlaying a base plate; wherein the base plate has an array of openings and solid portions located between and surrounding the openings; wherein the insert plate has an array of apertures; wherein the insert plate is axially shiftable relative to the base plate from a first state to a second state; wherein in the first state, the apertures align with the solid portions of the base plate with no overlap with the openings, and in the second state, the apertures at least partially align with the openings so as to at least partially overlap with the openings; wherein the base plate is chilled to a low temperature; wherein the base plate has at least two raised edges so that a plurality of assemblies can be stacked on top of each other with the raised edges of the base plates in physical contact;
(b) while the insert plate is in the first state, dispensing droplets of the pharmaceutical composition into the apertures to be supported by the solid portions of the base plate, wherein the droplets freeze on the base plate; repeating steps (a)-(b) multiple times and stacking the assemblies on top of each other to prepare a stack of assemblies with frozen droplets;
(c) placing the entire stack in a lyophilizer to dry the frozen droplets and produce lyospheres;
(d) providing a container nest comprising an array of pharmaceutically acceptable containers, each of the containers having a loading opening, wherein the openings in the base plate align with the loading openings of the containers; and
(e) dispensing the lyospheres into the containers by axially shifting the insert plate relative to the base plate to the second state; repeating steps (d)-(e) multiple times to dispense the lyospheres in each assembly into the containers in a container nest.

In other embodiments, the method of preparing lyospheres of a pharmaceutical composition comprises:
(a) providing an assembly comprising an insert plate overlaying a base plate; wherein the base plate has an array of openings and solid portions located between and surrounding the openings; wherein the insert plate has an array of apertures; wherein the insert plate is axially shiftable relative to the base plate from a first state to a second state; wherein in the first state, the apertures align with the solid portions of the base plate with no overlap with the openings, and in the second state, the apertures at least partially align with the openings so as to at least partially overlap with the openings; wherein the base plate is chilled to a low temperature; wherein the base plate has at least two raised edges so that a plurality of assemblies can be stacked on top of each other with the raised edges of the base plates in physical contact;
(b) while the insert plate is in the first state, dispensing droplets of the pharmaceutical composition into the apertures to be supported by the solid portions of the base plate, wherein the droplets freeze on the base plate; repeating steps (a)-(b) multiple times and stacking the assemblies on top of each other to prepare a stack of assemblies with frozen droplets;
(c) placing the entire stack in a lyophilizer to dry the frozen droplets and produce lyospheres; wherein the base plate is not in full contact with a shelf of the lyophilizer;
(d) providing a container nest comprising an array of pharmaceutically acceptable containers, each of the containers having a loading opening, wherein the openings in the base plate align with the loading openings of the containers; and
(e) dispensing the lyospheres into the containers by axially shifting the insert plate relative to the base plate to the second state; repeating steps (d)-(e) multiple times to dispense the lyospheres in each assembly into the containers in a container nest.

In yet other embodiments, the method of preparing lyospheres of a pharmaceutical composition comprises:
(a) providing an assembly comprising an insert plate overlaying a base plate; wherein the base plate has an array of openings and solid portions located between and surrounding the openings; wherein the insert plate has an array of apertures; wherein the insert plate is axially shiftable relative to the base plate from a first state to a second state; wherein in the first state, the apertures align with the solid portions of the base plate with no overlap with the openings, and in the second state, the apertures at least partially align with the openings so as to at least partially overlap with the openings; wherein the base plate is chilled to a low temperature; wherein first and second clips are edge mounted to the base plate and the insert plate with the insert plate overlaying the base plate;
(b) while the insert plate is in the first state, dispensing droplets of the pharmaceutical composition into the apertures to be supported by the solid portions of the base plate, wherein the droplets freeze on the base plate; repeating steps (a)-(b) multiple times and stacking the assemblies on top of each other to prepare a stack of assemblies with frozen droplets;
(c) placing the entire stack in a lyophilizer to dry the frozen droplets and produce lyospheres;
(d) providing a container nest comprising an array of pharmaceutically acceptable containers, each of the containers having a loading opening, wherein the openings in the base plate align with the loading openings of the containers; and
(e) dispensing the lyospheres into the containers by axially shifting the insert plate relative to the base plate to the second state; repeating steps (d)-(e) multiple times to dispense the lyospheres in each assembly into the containers in a container nest.

In still other embodiments, the method of preparing lyospheres of a pharmaceutical composition comprises:
(a) providing an assembly comprising an insert plate overlaying a base plate; wherein the base plate has an array of openings and solid portions located between and surrounding the openings; wherein the insert plate has an array of apertures; wherein the insert plate is axially shiftable relative to the base plate from a first state to a second state; wherein in the first state, the apertures align with the solid portions of the base plate with no overlap with the openings, and in the second state, the apertures at least partially align with the openings so as to at least partially overlap with the openings; wherein the base plate is chilled to a low temperature; wherein first and second clips are edge mounted to the base plate and the insert plate with the insert plate overlaying the base plate;
(b) while the insert plate is in the first state, dispensing droplets of the pharmaceutical composition into the apertures to be supported by the solid portions of the base plate, wherein the droplets freeze on the base plate; repeating steps (a)-(b) multiple times and stacking the assemblies on top of each other to prepare a stack of assemblies with frozen droplets;
(c) placing the entire stack in a lyophilizer to dry the frozen droplets and produce lyospheres; wherein the base plate is not in full contact with a shelf of the lyophilizer;
(d) providing a container nest comprising an array of pharmaceutically acceptable containers, each of the containers having a loading opening, wherein the openings in the base plate align with the loading openings of the containers; and
(e) dispensing the lyospheres into the containers by axially shifting the insert plate relative to the base plate to the second state; repeating steps (d)-(e) multiple times to dispense the lyospheres in each assembly into the containers in a container nest.

In still another aspect, provided herein is a method for preparing lyospheres of a pharmaceutical composition, comprising:
(a) providing an assembly comprising an insert plate overlaying a base plate; wherein the base plate has an array of openings and solid portions located between and surrounding the openings; wherein the insert plate has an array of apertures; wherein the insert plate is axially shiftable relative to the base plate from a first state to a second state; wherein in the first state, the apertures align with the solid portions of the base plate with no overlap with the openings, and in the second state, the apertures at least partially align with the openings so as to at least partially overlap with the openings; wherein the base plate is chilled to a low temperature;
(b) while the insert plate is in the first state, dispensing droplets of the pharmaceutical composition into the apertures to be supported by the solid portions of the base plate, wherein the droplets freeze on the base plate;
(c) placing the assembly in a lyophilizer to dry the frozen droplets and produce lyospheres;
(d) providing a container nest comprising an array of pharmaceutically acceptable containers, each of the containers having a loading opening;
(e) providing a dispensing funnel on top of the container nest, wherein the dispensing funnel has a support plate with an array of fill openings formed therethrough, wherein a first of the fill openings extends between a first top opening, formed in a top face of the support plate, to a bottom opening, formed in a bottom face of the support plate, wherein the top openings align with the openings in the base plate, wherein the bottom openings align with the loading openings of the containers; and
(f) dispensing the lyospheres into the containers by axially shifting the insert plate relative to the base plate to the second state.

In certain embodiments of the above method, wherein in (e) a second of the fill openings extends from a second top opening, formed in the top face of the support plate, to the first fill opening so as to merge therewith.

In some embodiments, the above method for preparing lyospheres of a pharmaceutical composition comprises: repeating steps (a)-(b) multiple times, preparing a stack of assemblies with a thermally conductive path formed between the assemblies; in step (c) drying the frozen droplets in the entire stack in a lyophilizer to produce lyospheres; then repeating steps (d)-(f) multiple times to dispense the lyospheres in each assembly into the containers in a container nest.

In one embodiment, the thermally conductive path is formed by stacking a plurality of assemblies on top of each other, wherein each base plate has at least two raised edges, and wherein the raised edges of the base plates are in physical contact. In certain embodiments, in step (c) the lowest base plate is not in full contact with a shelf of the lyophilizer.

In another embodiment, the thermally conductive path is formed by placing thermally conductive spacers along at least two edges of each base plate and stacking a plurality of assemblies on top of each other, wherein the spacers and the base plates are in physical contact. In certain embodiments, in step (c) the lowest base plate is not in full contact with a shelf of the lyophilizer.

In yet another embodiment, the thermally conductive path is formed by providing a thermally conductive rack with multiple levels and placing a plurality of assemblies on the levels of the rack, wherein the base plates and the levels of the rack are in physical contact. In certain embodiments, in step (c) the lowest base plate is not in full contact with a shelf of the lyophilizer.

In still another embodiment, the thermally conductive path is formed by edge mounting two thermally conductive clips to the base plate and the insert plate with the insert plate overlaying the base plate and stacking a plurality of assemblies on top of each other, wherein the clips are in physical contact. In certain embodiments, in step (c) the lowest base plate is not in full contact with a shelf of the lyophilizer.

In yet still another aspect, provided herein is a method for preparing lyospheres of a pharmaceutical composition comprising more than one formulation, comprising:
(a) providing an assembly comprising an insert plate overlaying a base plate; wherein the base plate has an array of openings and solid portions located between and surrounding the openings; wherein the insert plate has an array of apertures; wherein the insert plate is axially shiftable relative to the base plate from a first state to a second state; wherein in the first state, the apertures align with the solid portions of the base plate with no overlap with the openings, and in the second state, the apertures at least partially align with the openings so as to at least partially overlap with the openings; wherein the base plate is chilled to a low temperature;
(b) while the insert plate is in the first state, dispensing droplets of a first formulation into a first row of the apertures and droplets of a second formulation into a second row of the apertures to be supported by the solid portions of the base plate, wherein the droplets freeze on the base plate;
(c) placing the assembly in a lyophilizer to dry the frozen droplets and produce lyospheres;
(d) providing a container nest comprising an array of pharmaceutically acceptable containers, each of the containers having a loading opening;
(e) providing a dispensing funnel on top of the container nest, wherein the dispensing funnel has a support plate with an array of fill openings formed therethrough, wherein a first of the fill openings extends between a first top opening, formed in a top face of the support plate, to a bottom opening, formed in a bottom face of the support plate, wherein a second of the fill openings extends from a second top opening, formed in the top face of the support plate, to the first fill opening so as to merge therewith, wherein the first top openings align with a first row of the openings in the base plate and the second top openings align with a second row of the openings in the base plate, wherein the bottom openings align with the loading openings of the containers; and
(f) dispensing the lyospheres into the containers by axially shifting the insert plate relative to the base plate to the second state so that the first row of the apertures at least partially align with the first row of the openings so as to at least partially overlap with the first row of the openings and the second row of the apertures at least partially align with the second row of the openings so as to at least partially overlap with the second row of the openings.

In some embodiments, the method for preparing lyospheres of a pharmaceutical composition comprising more than one formulation, comprises: repeating steps (a)-(b) multiple times, preparing a stack of assemblies with a thermally conductive path formed between the assemblies; in step (c) drying the frozen droplets in the entire stack in a lyophilizer to produce lyospheres; then repeating steps (d)-(f) multiple times to dispense the lyospheres in each assembly into the containers in a container nest.

In one embodiment, the thermally conductive path is formed by stacking a plurality of assemblies on top of each other, wherein each base plate has at least two raised edges, and wherein the raised edges of the base plates are in physical contact. In certain embodiments, in step (c) the lowest base plate is not in full contact with a shelf of the lyophilizer.

In another embodiment, the thermally conductive path is formed by placing thermally conductive spacers along at least two edges of each base plate and stacking a plurality of assemblies on top of each other, wherein the spacers and the base plates are in physical contact. In certain embodiments, in step (c) the lowest base plate is not in full contact with a shelf of the lyophilizer.

In yet another embodiment, the thermally conductive path is formed by providing a thermally conductive rack with multiple levels and placing a plurality of assemblies on the levels of the rack, wherein the base plates and the levels of the rack are in physical contact. In certain embodiments, in step (c) the lowest base plate is not in full contact with a shelf of the lyophilizer.

In still another embodiment, the thermally conductive path is formed by edge mounting two thermally conductive clips to the base plate and the insert plate with the insert plate overlaying the base plate and stacking a plurality of assemblies on top of each other, wherein the clips are in physical contact. In certain embodiments, in step (c) the lowest base plate is not in full contact with a shelf of the lyophilizer.

In some embodiments of the method for preparing lyospheres of a pharmaceutical composition comprising more than one formulation, the first formulation is an active pharmaceutical ingredient (API) formulation and the second formulation is an adjuvant formulation.

In other embodiments of the method for preparing lyospheres of a pharmaceutical composition comprising more than one formulation, wherein the first formulation is a first API formulation and the second formulation is a second API formulation.

In some embodiments of the thermally conductive rack with multiple levels that can be used to form a thermal conductive path between multiple assemblies while lyophilizing frozen droplets, the lowest level of the rack is not in full contact with a shelf of the lyophilizer. In certain embodiments, the lowest level of the rack is in full contact with a shelf of the lyophilizer. In other embodiments, the lowest level of the rack is in full contact with a shelf of the lyophilizer, and a clip-style assembly is placed on the lowest level of the rack.

In one embodiment, the base plate surface is hydrophobic. The hydrophobic surface can comprise a chemically inert plastic such as polytetrafluoroethylene (PTFE), polypropylene, and the like. The hydrophobic surface can be bonded to a different material or simply comprise the top surface of a thin film made using the hydrophobic material (e.g., PTFE, polypropylene). To freeze the liquid droplet, the film containing the dispensed droplet is chilled to a temperature that is below the freezing point of the liquid composition.

In yet other embodiments of various methods disclosed herein, the temperature of the base plate at the droplet dispense and freezing step is below: about −70° C., about −80° C., about −90° C., about −120° C., about −150° C., about −180° C., or about −196° C. In some embodiments, the temperature of the base plate at the droplet dispense and freezing step is from about −70° C. to about −196° C., from about −70° C. to about −150° C. , from about −90° C. to about −196° C., from about −90° C. to about −130° C., from about −110° C. to about −150° C., from about −150° C. to about −196° C., from about −180° C. to about −196° C., or from about −180° C. to about −273° C. In one embodiment, the temperature of the base plate at the droplet dispense and freezing step is about −80° C. In another embodiment, the temperature of the base plate at the droplet dispense and freezing step is about −90° C. In yet another embodiment, the temperature of the base plate at the droplet dispense and freezing step is about −100° C. In still another embodiment, the temperature of the base plate at the droplet dispense and freezing step is about −115° C.

In certain embodiments of various methods disclosed herein, the droplets of the pharmaceutical composition are dispensed at a speed of: from about 0.5 mL/min to about 75 mL/min, from about 0.5 mL/min to about 50 mL/min, from about 5 mL/min to about 50 mL/min, from about 5 mL/min to about 40 mL/min, from about 10 mL/min to about 40 mL/min, or from about 10 mL/min to about 30 mL/min. In some embodiments, the droplets are dispensed at a speed of: about 0.5 mL/min, about 1 mL/min, about 2 mL/min, about 3 mL/min, about 5 mL/min, about 10 mL/min, about 15 mL/min, about 20 mL/min, about 25 mL/min, about 30 mL/min, about 35 mL/min, about 40 mL/min, about 45 mL/min, about 50 mL/min. In one embodiment, the droplets are dispensed at about 5 mL/min. In one embodiment, the droplets are dispensed at about 10 mL/min. In another embodiment, the droplets are dispensed at about 15 mL/min. In yet another embodiment, the droplets are dispensed at about 20 mL/min. In still another embodiment, the droplets are dispensed at about 25 mL/min. In yet still another embodiment, the droplets are dispensed at about 30 mL/min. In one embodiment, the droplets are dispensed at about 40 mL/min.

In some embodiments of various methods disclosed herein, the droplet is about 10 μL, about 15 μL, about 20 μL, about 25 μL, about 30 μL, about 40 μL, about 50 μL, about 75 μL, about 100 μL, about 125 μL, about 150 μL, about 175 μL, about 200 μL, about 225 μL, or about 250 μL. In other embodiments, the droplet is about 5-500 μL, about 10-250 μL, about 20-300 μL, about 20-150 μL, about 20-100 μL, about 30-100 μL, about 30-75 μL, about 20-50 μL, or about 20-30 μL. In one embodiment, the droplet is about 10 μL. In another embodiment, the droplet is about 20 μL. In yet another embodiment, the droplet is about 25 μL. In still another embodiment, the droplet is about 30 μL. In one embodiment, the droplet is about 35 μL. In another embodiment, the droplet is about 40 μL. In yet another embodiment, the droplet is about 45 μL. In still another embodiment, the droplet is about 50 μL. In still another embodiment, the droplet is about 60 μL. In still another embodiment, the droplet is about 100 μL.

In other embodiments of various methods disclosed herein, the distance from the open end of the dispensing tip to the base plate is: from about 0.05 cm to about 1 cm, from about 0.05 cm to about 0.8 cm, from about 0.05 cm to about 0.5 cm, from about 0.05 cm to about 0.3 cm, or from about 0.1 cm to about 0.3 cm. In some embodiments, the distance from the bottom of the dispensing tip to the base plate is: about 0.05 cm, about 0.1 cm, about 0.15 cm, about 0.2 cm, about 0.25 cm, about 0.3 cm, about 0.4 cm, about 0.5 cm, about 0.6 cm, about 0.7 cm, about 0.8 cm, about 0.9 cm, or about 1 cm. In one embodiment, the distance from the bottom of the dispensing tip to the base plate is about 0.1 cm. In another embodiment, the distance from the bottom of the dispensing tip to the base plate is about 0.2 cm. In yet another embodiment, the distance from the bottom of the dispensing tip to the base plate is about 0.3 cm.

In one specific embodiment, 10 μL droplets are dispensed at 0.6-1.5 mL/min with a distance of 0.17 cm. In another embodiment, 10 μL droplets are dispensed at 0.6-1.5 mL/min with a distance of 0.05 cm. In yet another embodiment, 20-100 μL droplets are dispensed at 0.5-10.0 mL/min with a distance of 0.05-0.3 cm. In still another embodiment, 20-100 μL droplets are dispensed at 0.5-10.0 mL/min with a distance of 0.1-0.3 cm. In one specific embodiment, 30-75 μL droplets are dispensed at 0.5-10.0 mL/min with a distance of 0.05-0.3 cm. In another embodiment, 30-75 μL droplets are dispensed at 0.5-10.0 mL/min with a distance of 0.1-0.3 cm. In yet another embodiment, 40-60 μL droplets are dispensed at 0.5-10.0 mL/min with a distance of 0.05-0.3 cm. In still another embodiment, 40-60 μL droplets are dispensed at 0.5-10.0 mL/min with a distance of 0.1-0.3 cm. In one specific embodiment, 20-100 μL droplets are dispensed at 0.5-3.0 mL/min with a distance of 0.05-0.3 cm. In another embodiment, 20-100 μL droplets are dispensed at 0.5-3.0 mL/min with a distance of 0.1-0.3 cm. In yet another embodiment, 30-75 μL droplets are dispensed at 0.5-3.0 mL/min with a distance of 0.05-0.3 cm. In still another embodiment, 30-75 μL droplets are dispensed at 0.5-3.0 mL/min with a distance of 0.1-0.3 cm. In one specific embodiment, 40-60 μL droplets are dispensed at 0.5-3.0 mL/min with a distance of 0.05-0.3 cm. In another embodiment, 40-60 μL droplets are dispensed at 0.5-3.0 mL/min with a distance of 0.1-0.3 cm. In yet another embodiment, 50 μL droplets are dispensed at 10.0-30.0 mL/min with a distance of 0.5-1.0 cm. In still another embodiment, 100 μL droplets are dispensed at 10.0-30.0 mL/min with a distance of 0.5-1.0 cm. In one embodiment, 150 μL droplets are dispensed at 10.0-30.0 mL/min with a distance of 0.5-1.0 cm. In another embodiment, 200 μL droplets are dispensed at 10.0-30.0 mL/min with a distance of 0.5-1.0 cm. In yet another embodiment, 250 μL droplets are dispensed at 10.0-30.0 mL/min with a distance of 0.5-1.0 cm.

In one specific embodiment, 50 μL droplets are dispensed at 1.2 mL/min with a distance of 0.16 cm. In another embodiment, 50 μL droplets are dispensed at 1.2 mL/min with a distance of 0.17 cm. In yet another embodiment, 50 μL droplets are dispensed at 0.6 mL/min with a distance of 0.17 cm. In still another embodiment, 50 μL droplets are dispensed at 10.0 mL/min with a distance of 0.32 cm. In one specific embodiment, 50 μL droplets are dispensed at 30.0 mL/min with a distance of 0.5 cm. In another embodiment, 100 μL droplets are dispensed at 24.0 mL/min with a distance of 0.5 cm. In yet another embodiment, 50 μL droplets are dispensed at 1.67 mL/min with a distance of 0.32 cm. In still another embodiment, 50 μL droplets are dispensed at 3.0 mL/min with a distance of 0.32 cm. In one specific embodiment, 100 μL droplets are dispensed at 1.5 mL/min with a distance of 0.1 cm. In another embodiment, 15 μL droplets are dispensed at 1.5 mL/min with a distance of 0.17 cm. In yet another embodiment, 50 μL droplets are dispensed at 1.2 mL/min with a distance of 0.26 cm. In still another embodiment, 50 μL droplets are dispensed at 0.6 mL/min with a distance of 0.27 cm. In one specific embodiment, 100 μL droplets are dispensed at 1.5 mL/min with a distance of 0.2 cm. In another embodiment, 15 μL droplets are dispensed at 1.5 mL/min with a distance of 0.27 cm. In yet another embodiment, 250 μL droplets are dispensed at 10.0 mL/min with a distance of 0.8 cm. In still another embodiment, 250 μL droplets are dispensed at 30.0 mL/min with a distance of 0.8 cm. In one specific embodiment, 250 μL droplets are dispensed at 30.0 mL/min with a distance of 1.0 cm. In another embodiment, 40 μL droplets are dispensed at 1.5 mL/min with a distance of 0.16 cm.

In certain embodiments of various methods disclosed herein, the base plate not being in full contact with a shelf of a lyophilizer can be achieved by placing one or more spacers between the base plate and the shelf. In other embodiments, the base plate not being in full contact with a shelf of a lyophilizer can be achieved by not placing any frozen droplets on the lowest base plate that is in full contact with the shelf.

The method disclosed herein can be utilized to prepare lyospheres of a variety of pharmaceutical compositions including biological materials (e.g., therapeutic proteins, cytokines, enzymes, antibodies, antigenic substances used in vaccines such as peptides and proteins) or chemical materials (e.g., small molecule compounds). In some embodiments, the pharmaceutical composition comprises a drug substance, a chemical compound, a therapeutic protein, an antibody, a vaccine, a fusion protein, a polypeptide, a peptide, a polynucleotide, a nucleotide, an antisense RNA, a siRNA, an oncolytic virus, a diagnostic, an enzyme, an adjuvant, an antigen, a virus, a virus-like particle, a prodrug, a toxoid, a vitamin, a lipid, a lipid nanoparticle, or a combination thereof. In one embodiment, the pharmaceutical composition comprises a drug substance. In one embodiment, the pharmaceutical composition comprises a chemical compound. In one embodiment, the pharmaceutical composition comprises a therapeutic protein. In one embodiment, the pharmaceutical composition comprises an antibody. In one embodiment, the pharmaceutical composition comprises a vaccine. In one embodiment, the pharmaceutical composition comprises a fusion protein. In one embodiment, the pharmaceutical composition comprises a polypeptide. In one embodiment, the pharmaceutical composition comprises a peptide. In one embodiment, the pharmaceutical composition comprises a polynucleotide. In one embodiment, the pharmaceutical composition comprises a nucleotide. In one embodiment, the pharmaceutical composition comprises an antisense RNA. In one embodiment, the pharmaceutical composition comprises a siRNA. In one embodiment, the pharmaceutical composition comprises an oncolytic virus. In one embodiment, the pharmaceutical composition comprises a diagnostic. In one embodiment, the pharmaceutical composition comprises an enzyme. In one embodiment, the pharmaceutical composition comprises an adjuvant. In one embodiment, the pharmaceutical composition comprises an antigen. In one embodiment, the pharmaceutical composition comprises a virus. In one embodiment, the pharmaceutical composition comprises a virus-like particle. In one embodiment, the pharmaceutical composition comprises a prodrug. In one embodiment, the pharmaceutical composition comprises a toxoid. In one embodiment, the pharmaceutical composition comprises a vitamin. In one embodiment, the pharmaceutical composition comprises a lipid. In one embodiment, the pharmaceutical composition comprises a lipid nanoparticle. In one embodiment, the pharmaceutical composition comprises a combination of two, three, four, five, six, seven, eight, nine, ten, or more selected from the list consisting of a chemical compound, a therapeutic protein, an antibody, a vaccine, a fusion protein, a polypeptide, a peptide, a polynucleotide, a nucleotide, an antisense RNA, a siRNA, an oncolytic virus, a diagnostic, an adjuvant, an antigen, a virus, a virus-like particle, a prodrug, a toxoid, a vitamin, a lipid, and a lipid nanoparticle. The pharmaceutical compositions can be useful in the fields of human health, veterinary health, medical science, laboratory science, or as a diagnostic.

The pharmaceutical composition is typically a liquid composition that also contains one or more components that confer stability on the biological or chemical material during storage of the liquid formulation, as well as during and after the freezing and lyophilization steps (for example, to preserve drying yield). Additional components that can be included as appropriate include but are not limited to pharmaceutically acceptable excipients, additives, diluents, buffers, sugars, amino acids (such as histidine, glycine, glutamine, asparagine, arginine, or lysine), chelating agents, surfactants, polyols, bulking agents, stabilizers, cryoprotectants, lyoprotectants, solubilizers, emulsifiers, salts, adjuvants, tonicity enhancing agents (such as alkali metal halides, preferably sodium or potassium chloride, mannitol, sorbitol), delivery vehicles, and anti-microbial preservatives. Acceptable formulation components for pharmaceutical preparations are nontoxic to recipients at the dosages and concentrations employed.

The lyospheres prepared by the methods disclosed herein can be easily integrated into a variety of dosage sizes by choosing the volume of the droplet and the number of lyospheres added to a single or multiple dosage container or delivery device. Also, the methods readily enable the preparation of combination therapeutic or immunogenic products, in which lyospheres comprising one material are combined in a single container with lyospheres comprising a different material. For example, lyospheres prepared from different antigen compositions, such as measles, mumps, rubella, and varicella, can be combined in a single container to obtain a multi-component vaccine. This allows the different antigens to remain separate until reconstitution, which can increase shelf-life of the vaccine. Similarly, combination products can contain separate antigen-comprising lyospheres and adjuvant-comprising lyospheres. Another example is a combination of lyospheres comprising a protein with lyospheres comprising a peptide.

Lyospheres prepared using methods described herein can be dispensed into containers in nests or tubs. In these nests or tubs, a number of vials or pre-filled syringes (for example, 100, 120, or some other number of containers) are arranged in precise, known positions relative to each other and in a holder that can be easily moved by automation equipment. Any commercially available nests or tubs can be used with the methods and assemblies described herein. Examples of such nests or tubs include but are not limited to adaptiQ® vials (Schott AG, Mainz, Germany), EZ-fill® syringes, vials and cartridges (Stevanato Group/OMPI, Padua, Italy), Gx® RTF vial (nest & tub) (Gerresheimer Glass Inc. Vineland, N.J., USA), D2F glass vials and prefillable syringes (Nipro, Mechelen, Belgium), BD Hypak pre-fillable syringes (BD Medical—Pharmaceutical Systems, N.J., USA.).

In yet still another aspect, provided herein is a container containing a lyosphere of a pharmaceutical composition, wherein the lyosphere is prepared by various methods described herein. Some preferred containers include vials, glass vials, cartridges, dual chamber cartridges, multi chamber cartridges, syringes, and pre-fillable syringes, etc. The container can be any commercially available containers in nests or tubs, including but not limited to adaptiQ® vials (Schott AG, Mainz, Germany), EZ-fill® syringes, vials and cartridges (Stevanato Group/OMPI, Padua, Italy), Gx® RTF vial (nest & tub) (Gerresheimer Glass Inc. Vineland, N.J., USA), D2F glass vials and prefillable syringes (Nipro, Mechelen, Belgium), BD Hypak pre-fillable syringes (BD Medical—Pharmaceutical Systems, N.J., USA.).

In one embodiment, the container contains a lyosphere of a pharmaceutical composition, wherein the lyosphere is prepared by:
(a) providing an assembly comprising an insert plate overlaying a base plate; wherein the insert plate has an array of apertures; wherein the insert plate is axially shiftable relative to the base plate from a first state to a second state; wherein in the first state, the apertures align with the solid portions of the base plate with no overlap with the openings, and in the second state, the apertures at least partially align with the openings so as to at least partially overlap with the openings; wherein the base plate is chilled to a low temperature;
(b) while the insert plate is in the first state, dispensing droplets of the pharmaceutical composition into the apertures to be supported by the solid portions of the base plate, wherein the droplets freeze on the base plate;
(c) placing the assembly in a lyophilizer to dry the frozen droplets and produce lyospheres;
(d) providing a container nest comprising an array of pharmaceutically acceptable containers, each of the containers having a loading opening, wherein the openings in the base plate align with the loading openings of the containers; and
(e) dispensing the lyospheres into the containers by axially shifting the insert plate relative to the base plate to the second state.

In another embodiment, the container contains more than one lyospheres, wherein each lyosphere is prepared by various methods described herein. In yet another embodiment, the container contains more than one lyospheres, wherein each lyosphere is prepared by various methods described herein, and wherein each lyosphere is a lyosphere of a different pharmaceutical composition. In still another embodiment, the container contains two, three, four, five, six, seven, eight, nine, ten, twenty, thirty, or more lyospheres, wherein each lyosphere is prepared by various methods described herein. In some embodiments, the two, three, four, five, six, seven, eight, nine, ten, twenty, thirty, or more lyospheres are lyospheres of the same pharmaceutical composition. In other embodiments, the two, three, four, five, six, seven, eight, nine, ten, or more lyospheres are lyospheres of different pharmaceutical compositions.

### Assemblies and Systems for Preparing and/or Dispensing Lyospheres of Pharmaceutical Compositions

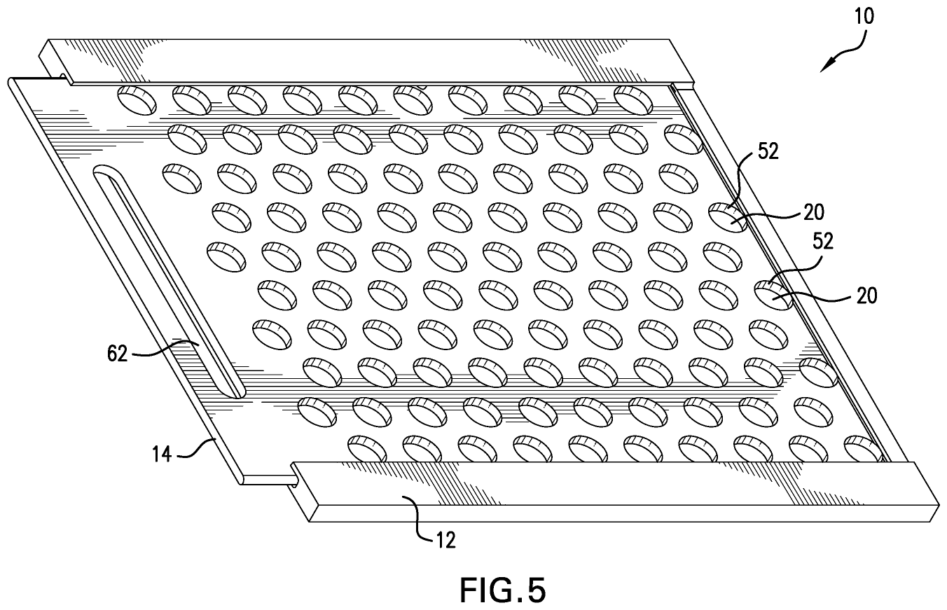

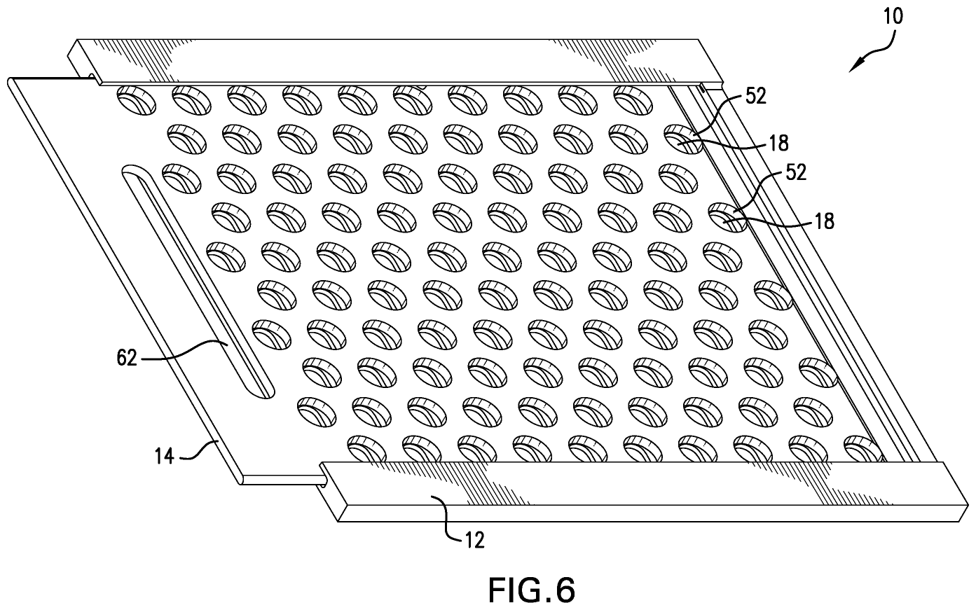

An assembly 10 is provided herein for preparing and/or dispensing lyospheres in accordance with any of the methods described above. With reference to FIGS. 5 and 6, the assembly 10 generally includes a base plate 12 and an insert plate 14. The assembly 10 may be used as a stand-alone device in preparing and/or dispensing lyospheres, but may be also incorporated into systems, as described below, for preparing lyospheres and/or dispensing lyospheres. Within these systems, a plurality of the assemblies 10 may be utilized.

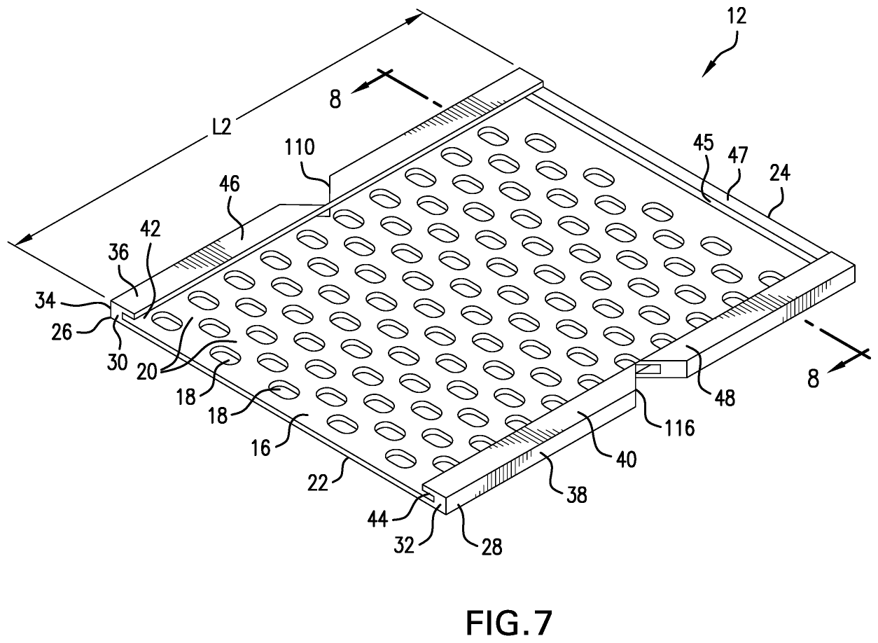

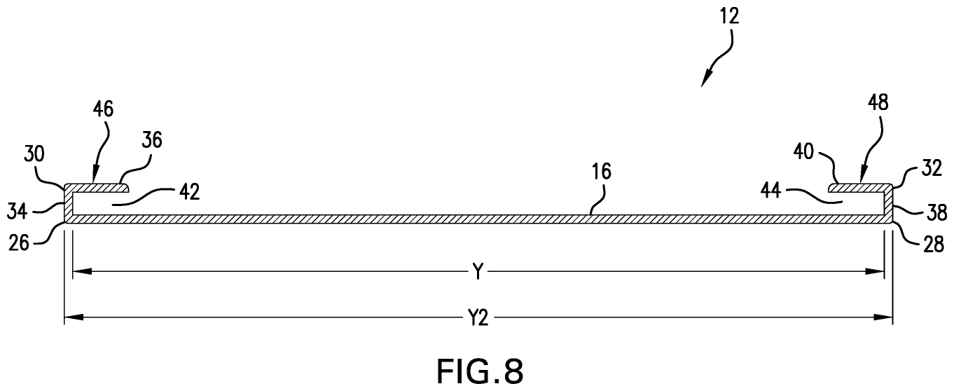

With reference to FIGS. 7 and 8, the base plate 12 includes a generally planar base 16 with an array of openings 18 formed therethrough. The openings 18 are spaced apart such that solid portions 20 of the base 16 are located between, and surround, the openings 18. The openings 18 may be arranged in an array of staggered rows, so that the openings 18 of alternating rows are aligned in phase. As will be recognized by those skilled in the art, the openings 18 may be arranged in various arrays, including, to varying extents, parallel rows (in-phase or staggered), parallel arcs (in-phase or staggered), and/or irregular patterns.

The assembly 10, including the base plate 12, may be exposed to extremely low temperatures, e.g., in the range of −70° C. to −180° C., to prepare the lyospheres. Thermal conductivity of the base plate 12 is important at these low temperatures, particularly to impart uniformity to the lyospheres being prepared. In addition, as will be described below, relative movement between the base plate 12 and the insert plate 14 is required. Thus, the maintenance of the general planarity of the base 16, i.e., resistance to warping, is important. These criteria can be addressed through material selection and design, particularly the thickness of the base plate 12. It is preferred that the base plate 12 be unitarily formed of a metallic material, such as stainless steel, aluminum, titanium, and/or copper, including alloys and combinations thereof (e.g., a layered structure including layers of different materials). It is preferred that the base plate 12 be formed of a material having a minimum thermal conductivity of 10 W/m*K.

In addition, it is preferred that the base plate 12 be generally rectangular with spaced-apart first and second ends 22, 24 and spaced-apart first and second side edges 26, 28 which extend between the first and second ends 22, 24. The first and second side edges 26, 28 can be the same length as each of the first and second ends 22, 24. In a preferred embodiment, the first and second side edges 26, 28 are greater in length than each of the first and second ends 22, 24. As a result, the base 16 may be also generally rectangular, being bounded by the first and second ends 22, 24 and the first and second side edges 26, 28.

A first upstanding channel 30 extends along the first side edge 26, with a second upstanding channel 32 extending along the second side edge 28. The first and second upstanding channels 30, 32 are configured to receive the insert plate 14 in sliding engagement to guide axial movement of the insert plate 14 relative to the base plate 12.

As shown in FIGS. 7 and 8, the first and second upstanding channels 30, 32 may be formed integrally with the base 16. In this embodiment, the first upstanding channel 30 may include a first wall 34 extending upwardly from the first side edge 26, with a second wall 36 extending transversely from the first wall 34 spaced from, in overlapping relation to, the base 16. The second upstanding channel 32 may include a third wall 38 extending upwardly from the second side edge 28, with a fourth wall 40 extending transversely from the third wall 38 spaced from, in overlapping relation to, the base 16. With this arrangement, open portion 42 of the first upstanding channel 30 is in facing arrangement with open portion 44 of the second upstanding channel 32. In addition, upper surface 46 of the second wall 36 is generally coplanar with upper surface 48 of the fourth wall 40 so as to collectively define a common resting surface for a second assembly placed atop the assembly 10 in a stacked fashion. The base plate 12 may be extruded, bent or otherwise machined from a blank, assembled from multiple components (e.g., welded components), in forming the first and second upstanding channels 30, 32 integral with the base 16.

An upstanding stop wall 45 may be provided along the second end 24 to limit movement of the insert plate 14 relative to the base plate 12. The stop wall 45 may include a lip 47 which extends towards the first end 22. The lip 47 is positioned to be above the insert plate 14 when received within the first and second upstanding channels 30, 32. In this manner, the lip 47 may act as a catch limiting upward separation of the insert plate 14 from the base plate 12. Preferably, the lip 47 has an upper surface 49 which is located below the upper surfaces 46, 48 (i.e., the upper surface 49 is located closer to the base 16 than the upper surfaces 46, 48).

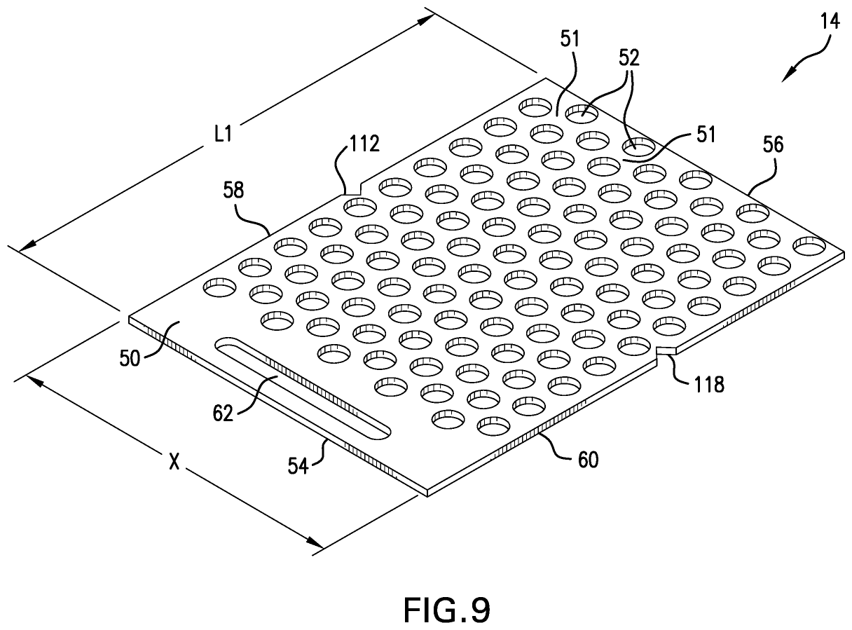

With reference to FIG. 9, the insert plate 14 includes a generally planar body 50 with an array of apertures 52 formed therethrough. The apertures 52 are spaced apart such that solid portions 51 of the insert plate 14 are located between, and surround, the apertures 52. The apertures 52 may be arranged in an array of staggered rows, so that the apertures 52 of alternating rows are aligned in phase. As will be recognized by those skilled in the art, the apertures 52 may be arranged in various arrays, including, to varying extents, parallel rows (in-phase or staggered), parallel arcs (in-phase or staggered), and/or irregular patterns.

The body 50 is generally rectangular with spaced-apart first and second plate ends 54, 56 and spaced-apart first and second plate side edges 58, 60 which extend between the first and second plate ends 54, 56. The first and second plate side edges 58, 60 can be the same length as each of the first and second plate ends 54, 56. In a preferred embodiment, the first and second plate side edges 58, 60 are greater in length than each of the first and second plate ends 54, 56. The distance X between the first and second plate side edges 54, 56 is less than the distance Y between the first wall 34 and the third wall 38 of the base plate 12 so that the insert plate 14 may be inserted therebetween. It is preferred that the clearances between the insert plate 14 and the first wall 34 and the third wall 38 be kept to a minimum to minimize play between the insert plate 14 and the base plate 12.

The insert plate 14 may have a length L1 between the first and second plate ends 54, 56 which is greater than length L2 between the ends 22, 24 of the base plate 12. As such, the insert plate 14, while atop the base plate 12, may extend therefrom. A handle opening 62 may be formed in the insert plate 14, adjacent to the first plate end 54, positioned to not overlap with the base plate 12 with the insert plate 14 being atop the base plate 12, The handle opening 62 may be configured to receive a user's hand or automated handling component (such as a robotic gripper) in grasping the insert plate 14 for handling, including to facilitate the axial shifting of the insert plate 14 relative to the base plate 12.

The insert plate 14 is preferably formed from a polymeric material, such as polyethylene, polyethylene terephthalate, polypropylene, polyoxymethylene, polycarbonate, polyetherimide, polytetrafluoroethylene, polyvinylidene fluoride, perfluoroalkoxy polymer, fluorinated ethylene propylene. It is preferred that the insert plate 14 be formed of a material having low friction and good wear properties. In a specific embodiment, the insert plate is formed from Delrin.

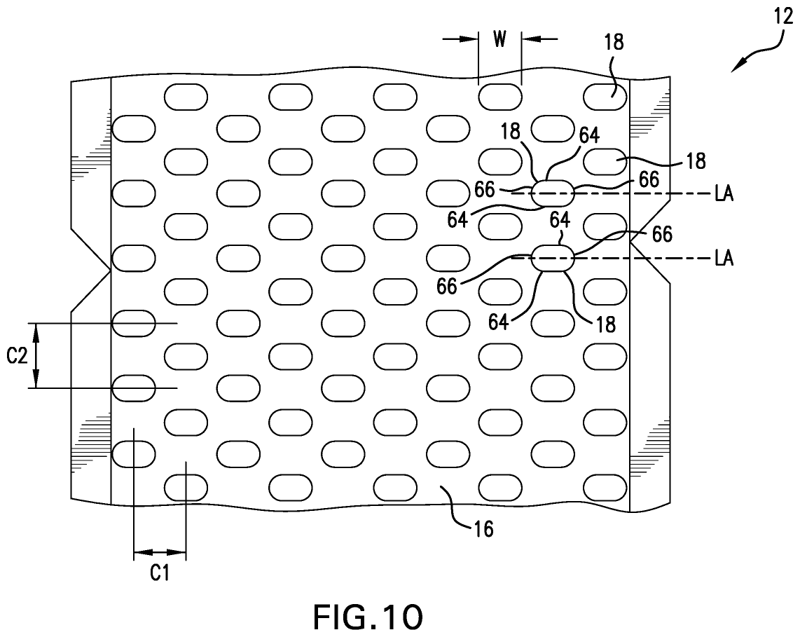

As shown in FIG. 10, preferably, the openings 18 in the base plate 12 are all similarly formed and are preferably oval shaped. With an oval shape, the openings 18 are each elongated about a longitudinal axis LA with spaced-apart straight sides 64 extending between ends 66, which may be arcuate to provide the oval shape.

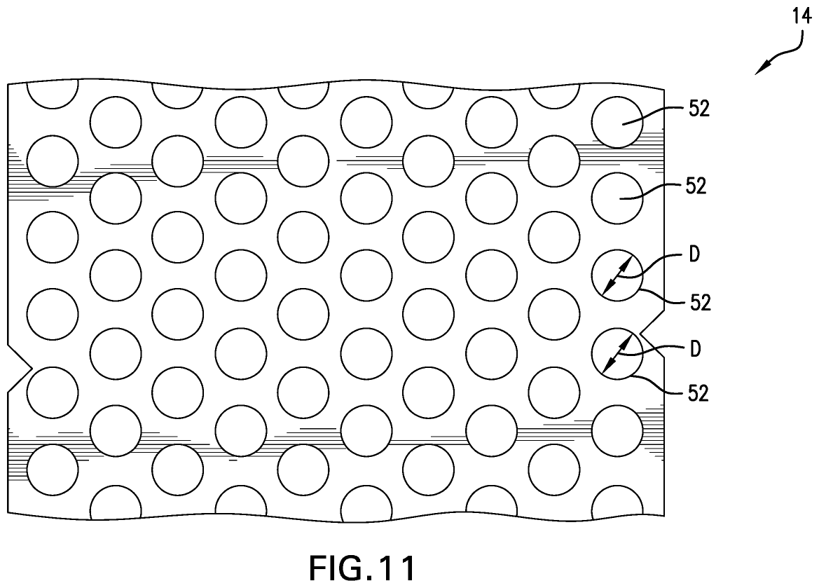

As shown in FIG. 11, preferably, the apertures 52 in the insert plate 14 are all similarly formed and are preferably circular, with a diameter D. It is preferred that the length W of the openings 18 (as measured between the ends 66 (FIG. 10)) be greater than or equal to the diameter D of the apertures 52.

In an assembled state, as shown in FIGS. 5 and 6, the insert plate 14 overlays the base 16 of the base plate 12, with the insert plate 14 being inserted into the first and second upstanding channels 30, 32. The insert plate 14 is axially shiftable relative to the base plate 12 from a first state (FIG. 5) to a second state (FIG. 6). In the first state, the apertures 52 are aligned with the solid portions 20 of the base plate 12 with no overlap between the apertures 52 and the openings 18. In the second state, the apertures 52 are at least partially aligned with the openings 18 so as to at least partially overlap the openings 18. The insert plate 14 is preferably axially shifted relative to the base plate 12 between the first and second states in a direction along a longitudinal axis extending between the first and second ends 22, 24 of the base plate 12. The first and second upstanding channels 30, 32 are configured to guide the insert plate 14 during the axial shifting relative to the base plate 12, particularly in constraining the movement to generally one degree of freedom (i.e., limiting movement along one directional axis). The openings 18 and the apertures 52 are sized and located to be in and out of alignment, respectively, between the first and second states.

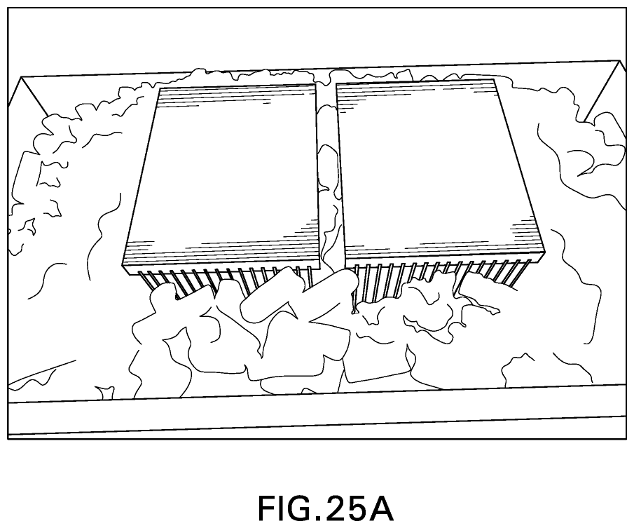

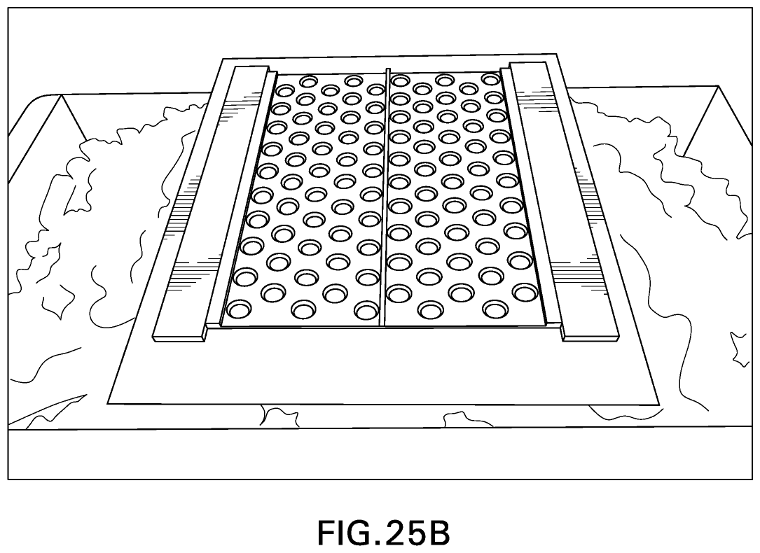

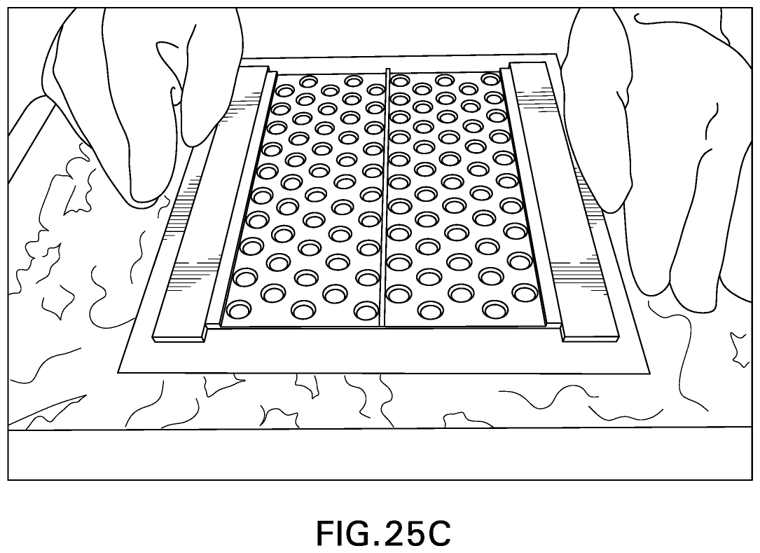

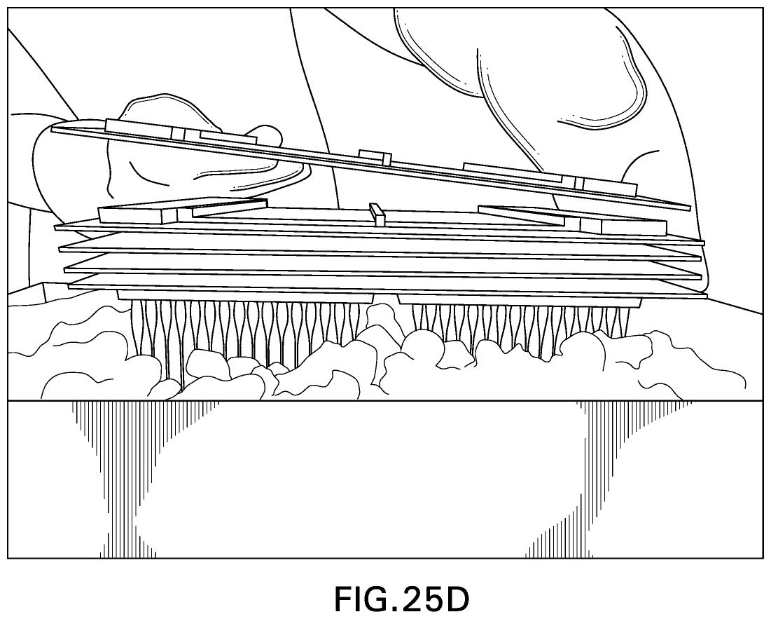

In the first state, the base plate 12 provides a support surface for droplets of liquid composition of a drug product to be disposed thereon within each of the apertures 52 to be frozen, in accordance with any of the methods described above. After the droplets have frozen (FIG. 25C), the assembly 10 is then placed into a lyophilizer to dry the frozen droplets to prepare the lyospheres. Once lyophilized, for subsequent dispensing, the insert plate 14 is axially shifted relative to the base plate 12 to the second state, with the lyospheres passing from the apertures 52 and through the openings 18 to a collection area or one or more containers located below the assembly 10.

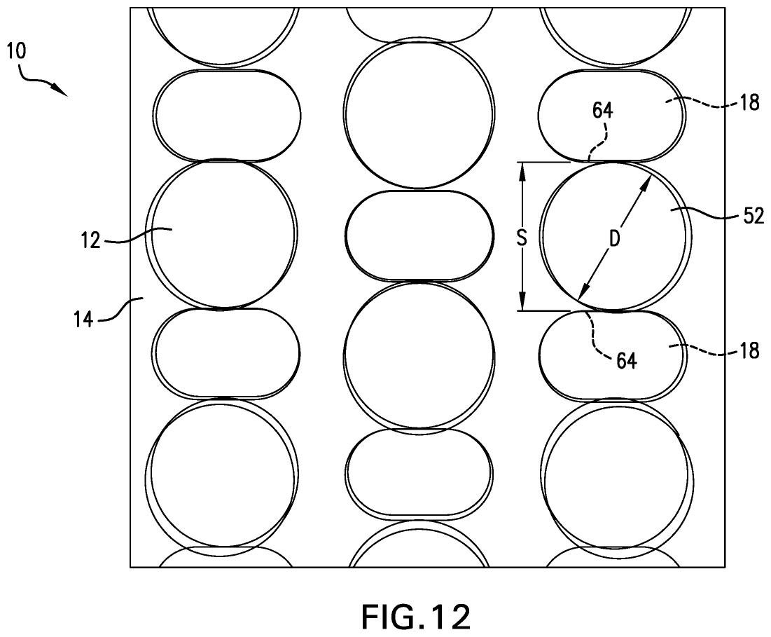

To ensure that the droplets are retained within the apertures 52, as shown in FIG. 12, it is preferred that the apertures 52 be located wholly between the openings 18 in the first state. This provides for the apertures 52 to be fully in overlapping alignment with the solid portions 20 of the base 16 of the base plate 12. In one configuration, the spacing S between the straight sides 64 of a pair of adjacent openings 18 may be slightly greater that the diameter D of the apertures 52.

It is also noted that the base plate 12 and the insert plate 14 may be formed of different materials, having different thermal properties. Accordingly, the base plate 12 and the insert plate 14 may contract at different rates and amounts when exposed to the low temperatures utilized during freezing and lyophilization. It is preferred that the base plate 12 and the insert plate 14 be formed of materials which will provide the insert plate 14 with more contraction than the base plate 12 at low temperatures. This will cause the apertures 52 to contract more than the openings 18 in ensuring that the apertures 52 remain out of alignment with the openings 18 in the first state. If the base plate 12 were provided with more contraction than the insert plate 14, it would be possible that the apertures 52 may come into partial alignment with the openings 18.

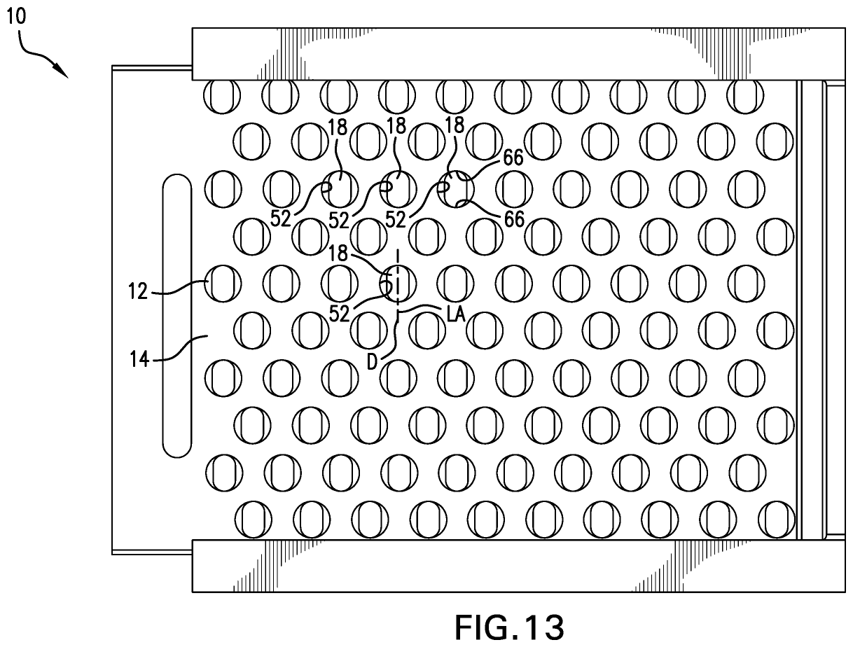

Preferably, in the second state, as shown in FIG. 13, the diameter D of each of the apertures 52 is generally coaxial with the longitudinal axis LA of the opening 18 aligned therewith. Moreover, in the second state, it is preferred that the diameter D of each of the apertures 52 be equal to or greater than the maximum length between the ends 66 of the opening 18 aligned therewith. In this manner, each of the apertures 52 spans at least the full width of the opening 18 aligned therewith, along the respective longitudinal axis LA. It is further preferred that the area defined by each of the apertures 52 (e.g., based on the diameter D) be greater than the area defined by each of the openings 18 (e.g., area based on the oval shape).

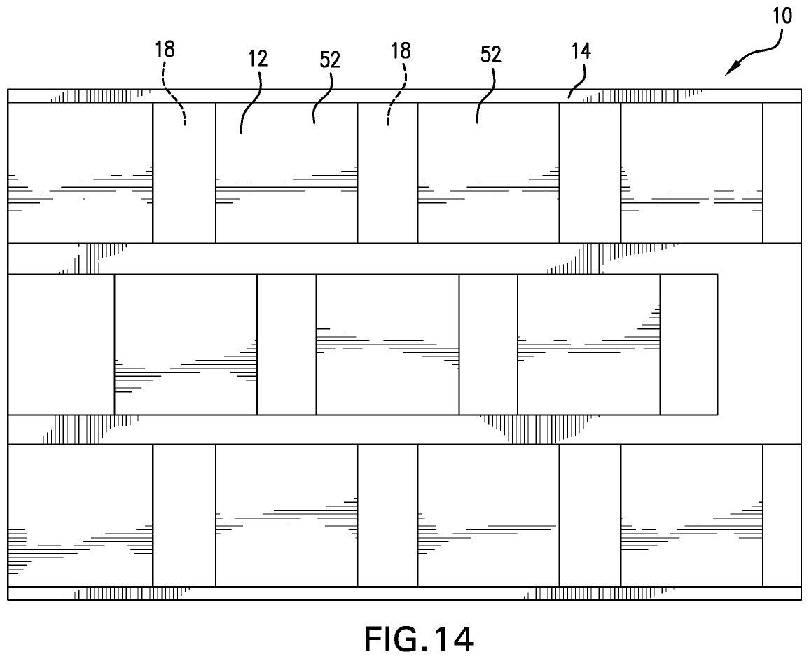

As will be appreciated by those skilled in the art, the openings 18 and the apertures 52 may be provided with various configurations, such as being rectangular. For example, with reference to FIG. 14, the openings 18 may be formed rectangular with the ends 66 being provided as straight, rather than as arcuate. The apertures 52 may be also formed rectangular having a width D, as measured in the direction of shifting, with the straight sides 64 of adjacent pairs of the openings 18 being separated by the spacing S which is slightly greater that the width D.

As discussed below, the assembly 10 may be used to dispense the lyospheres into vials or other containers. The openings 18 may be located to have center-to-center distances to accommodate a tub, nest or tray of vials, syringes, or other containers intended to receive the lyospheres. With reference to FIG. 10, the openings 18 may be located to have a first center-to-center spacing C1, along one coordinate direction, and a second center-to-center spacing C2, along a second coordinate direction. One of the spacings, such as C2, may be aligned in the axial shifting direction of the insert plate 14. The size of the openings 18 may be also defined by the intended containers. In addition, the quantity and array of the openings 18 may be dictated by the intended containers, particularly if the intended containers are provided in a fixed tray or nest with the openings of the intended containers defining a fixed pattern. With the spacing, size, quantity, and arrangement of the openings 18 being determined, details of the apertures 52 may be determined. It is preferred that the apertures 52 be provided in one-to-one correspondence with the openings 18 (i.e., provided in the same quantity). The arrangement, spacing and size of the apertures 52 may be determined based on ensuring that the first and second states, as described above, may be achieved. In other words, the apertures 52 are preferably sized and spaced to align in the first state with the solid portions 20 of the base 16, with any configuration (size, arrangement, spacing, shape, quantity) of the openings 18, and to be in at least partial alignment with the openings 18 in the second state.

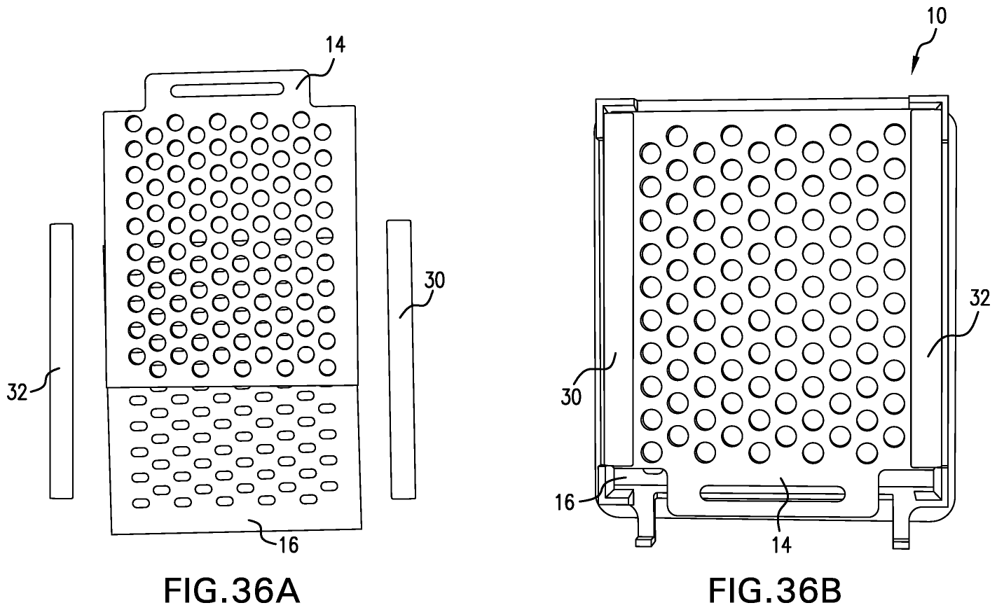

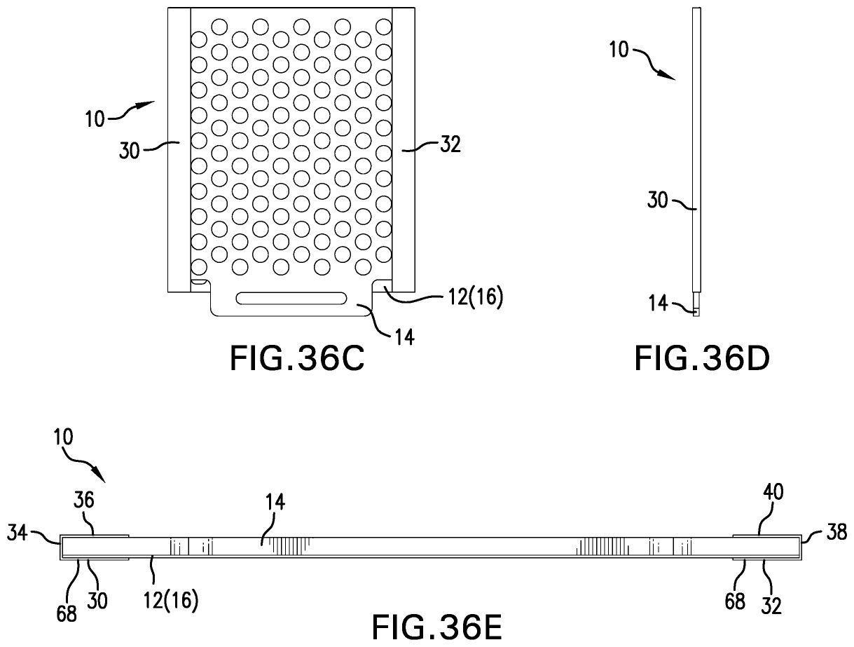

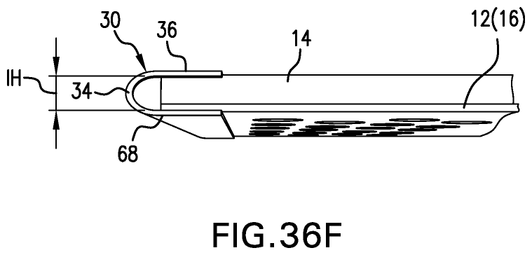

Alternatively, as shown in FIGS. 36A-36F, the first and second channels 30, 32 may be provided as separate clips mountable to the base 16 with the insert plate 14 overlaid thereon. With this arrangement, the base plate 12 is simplified to only include the base 16. FIG. 36A shows the insert plate 14, the base 16, and the first and second channels 30, 32 as components, with FIG. 36B showing noted components assembled in forming the assembly 10. The first and second channels 30, 32 and the base 16 may be provided with the same length, which may be greater than portions of the insert plate 14 so as to protrude therefrom, as shown in FIGS. 36C-36D. The lengths of the components may be varied, including, for example, to have the base 16 with a greater length than the first and second channels 30, 32 and greater length than portions of the insert plate 14.

As shown in FIG. 36E, the first and second channels 30, 32 may be each provided as a clip formed for edge mounting onto the insert plate 14 and the base 16 in a stacked arrangement. In particular, for the first channel 30, the second wall 36 is elastically bendable relative to the first wall 34, so that the second wall 36, upon being urged outwardly is inwardly biased under the force of memory urging the second wall 36 to return to its original rest state. The same arrangement may be provided for the second channel 32, with the fourth wall 40 being elastically bendable relative to the third wall 38. Both the first channel 30 and the second channel 32 may be provided with an additional base wall 68 which is also elastically bendable relative to the first wall 34 and the third wall 38, respectively, in the same manner as the second wall 36 and the fourth wall 40. The first and second channels 30, 32, with this configuration, may have a U-shaped cross-section with the corners being preferably rounded, as shown representatively in FIG. 36F relative to the first channel 30 (it being understood that the second channel 32 may be similarly formed). Alternatively, the corners may be formed non-rounded (e.g., intersecting planar surfaces).

To ensure that the first and second channels 30, 32 may be properly mounted to the base 16 and the insert plate 14 in a stacked arrangement, the first wall 34 and the third wall 38, respectively, may be each provided with an inner height IH which is at least as great as the height of the stack of the insert plate 14 and the base 16. The inner height IH may be defined along an inner surface of the first wall 34 and the third wall 38, which faces the stack of the insert plate 14 and the base 16, between inner corners where joined with the second wall 36/fourth wall 40, respectively and the base wall 68.

As shown in FIGS. 36C-36E, in an assembled state, the first and second channels 30, 32 apply clamping force to the insert plate 14 and the base 16 to maintain the stack arrangement thereof. In addition, portions of the first and second channels 30, 32, overlap the insert plate 14 and the base 16, so that these portions will provide supporting surfaces when stacking the assembly 10 with other assemblies. In particular, the base walls 68, overlap the base 16 and, define a lower resting surface for the assembly 10, while the second wall 36 of the first channel 30 and the fourth wall 40 of the second channel 32, overlap the insert plate 14 and, define an upper resting surface for any assembly 10 stacked thereupon. Advantageously, the first and second channels 30, 32 each define a continuous thermal conductive path between these surfaces, which passes around the stack of the insert plate 14 and the base 16, via the first wall 34 and the third wall 38, respectively. With a stack of the assemblies 10, continuous thermal pathways may be defined along edges of the stack of the assemblies 10.

The first and second channels 30, 32, may be metallic (e.g., aluminum), formed as a unitary body, e.g., by extrusion, bending of a blank, and so forth. Good thermal conductivity is desired for the first and second channels 30, 32. In addition, the base 16 may be metallic (e.g., stainless steel), formed as a unitary body, e.g., machined from sheet metal (e.g., with the openings 18 being stamped or die cut). The insert plate 14 may be formed of a polymeric material and formed using any known technique (e.g., with plastic sheeting being perforated to form the apertures 52).

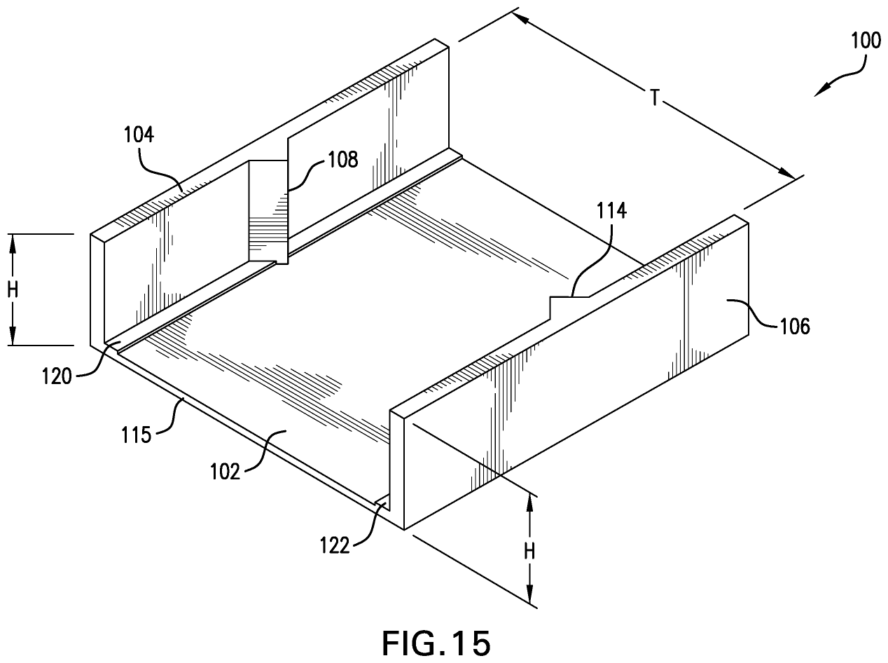

As a further embodiment, the assembly 10 may be used within a system utilizing a carrier 100. As shown in FIG. 15, the carrier 100 includes a bottom plate 102, a first upstanding wall 104, and a second upstanding wall 106. The first and second upstanding walls 104, 106 are sufficiently spaced apart to accommodate the assembly 10 therebetween. It is preferred that the spacing T between the first and second upstanding walls 104, 106 be slightly greater than the width Y2 of the base plate 12 (i.e., distance between the first and second side edges 26, 28 (FIG. 8)). With this arrangement, a plurality of assemblies 10 may be stacked between the first and second upstanding walls 104, 106, as shown in FIG. 16.

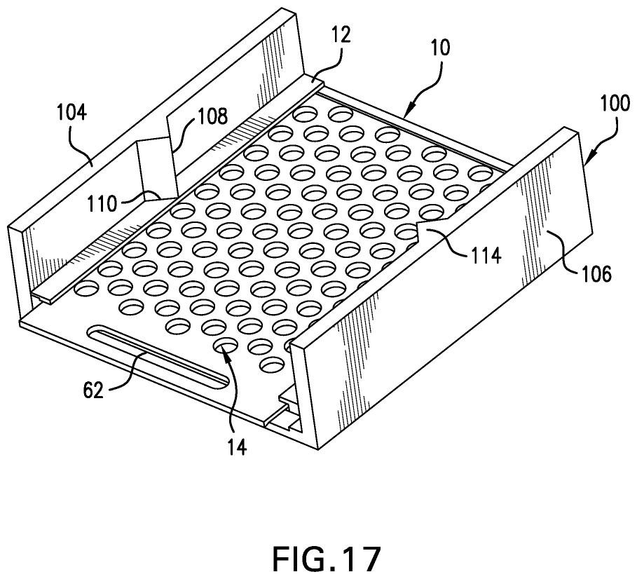

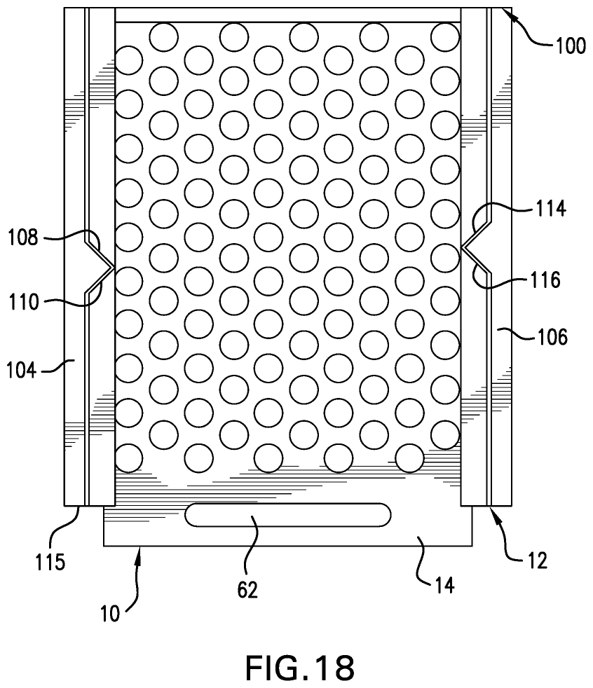

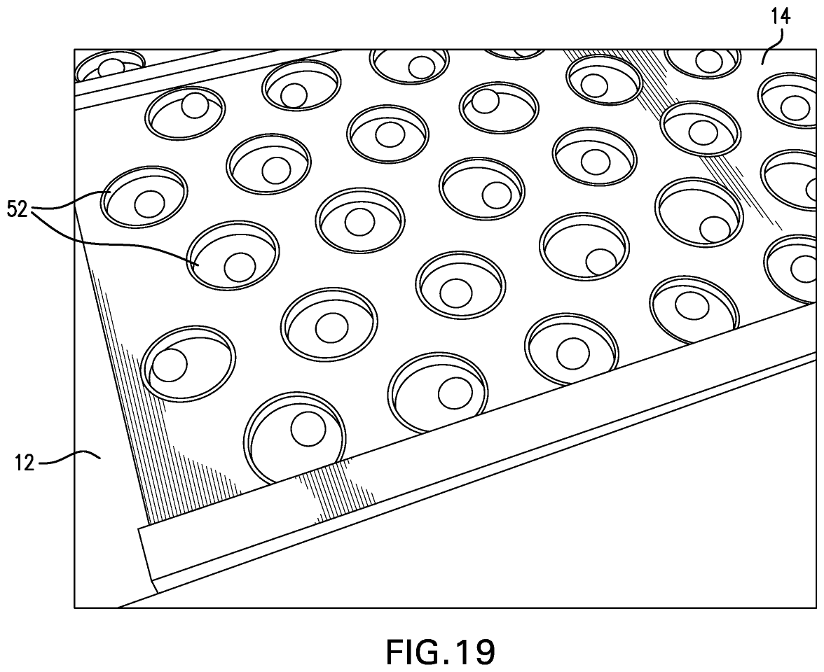

The bottom plate 102 may be similarly dimensioned to the base 16 of the base plate 12. With this dimensioning, the insert plates 14 of the stacked assemblies 10 may extend from the carrier 100 with the handle openings 62 thereof being exposed for handling, as shown in FIGS. 17 and 18.

To provide retention of the assembly 10 in the carrier 100, and to restrict relative movement between the base plate 12 and the insert plate 14, a first retention prism 108 protrudes inwardly from the first upstanding wall 104. Correspondingly, a first notch 110 may be formed on the base plate 12 configured to shape-matingly receive the first retention prism 108 with the assembly 10 being accommodated in the carrier 100. The inter-engagement between the first retention prism 108 and the first notch 110 inhibits axial movement of the base plate 12 relative to the carrier 100. In addition, the insert plate 14 may include a first plate notch 112 formed to align with the first notch 110 and configured to also shape-matingly receive the first retention prism 108 with the assembly 10 being accommodated in the carrier 100. The inter-engagement between the first retention prism 108 and the first plate notch 112 inhibits axial movement of the insert plate 14 relative to the carrier 100. The first retention prism 108 is formed to extend upwardly from the bottom plate 102 to allow for stacking of the assemblies 10 within the carrier 100 with subsequent inhibition of relative movement of the base plates 12 and the insert plates 14 of the stacked assemblies 10. This allows for frozen droplets to be prepared on the assemblies 10 and stacked in the carrier 100 with the assemblies 10 being in the first state. The carrier 100, with the stacked assemblies 10, is placed into a lyophilizer to lyophilize the frozen droplets to ease handling. The assemblies 10 remain in the first state during lyophilization.

A second retention prism 114 may be provided to protrude inwardly from the second upstanding wall 106. The base plate 12 may have a second notch 116 and the insert plate may have a second plate notch 118, each formed to shape-matingly receive the second retention prism 114 to inhibit axial movement of the base plate 12 and the insert plate 14 relative to the carrier 100. The second retention prism 114 preferably extends upwardly from the bottom plate 102.

The first retention prism 108 and the second retention prism 114 may be out of axial alignment (i.e., located at different distances from a front edge 115 of the carrier 100). This allows for stacking of the assemblies 10 in one orientation to ensure that the handles 62 are along one end (e.g., the front edge 115) of the carrier 100. In addition, or alternatively, the first and second retention prisms 108 and 114 may have the same or different profiles. The first and second retention prisms 108, 114 may have polygonal profiles (such as triangular as shown in the FIGS. 15-18), arcuate profiles, and/or irregular profiles.

The first and second retention prism 108, 114 need not be provided on the carrier 100. As shown in FIGS. 5 and 6, the assemblies 10 may be provided without notches.

The heights H of the first and second upstanding walls 104, 106 may be selected such that an accommodated stack of the assemblies 10 protrudes above the first and second upstanding walls 104, 106. This allows for an upper shelf of a lyophilizer to press against the stack of assemblies 10 in causing compression thereof without hinderance of the first and second upstanding walls 104, 106. The compressive force also presses the carrier 100 against a lower shelf of the lyophilizer. This compression allows for best contact between the upper and lower shelves, the assemblies 10, and the carrier 100, to allow for good thermal conduction therebetween during lyophilization. Optionally, a top lid can be provided that can rest on top of the uppermost assembly.

The carrier 100 may be assembled from multiple fabricated components or may be unitarily fabricated (such as by three-dimensional manufacturing). The carrier 100 may be formed from metallic material, such as stainless steel, aluminum, titanium, and/or copper, including alloys and combinations thereof (e.g., a layered structure including layers of different materials).

In use, once loaded with the assemblies 10, the carrier 100 may be placed into a lyophilizer, particularly to rest on a temperature-controlled shelf An upper temperature-controlled shelf may be pressed down onto the top of the stack of assemblies 10. As a result, as shown in FIGS. 2A-2C, the assemblies 10 are sandwiched between the two temperature-controlled shelves of the lyophilizer for lyophilization of the frozen pellets to form lyospheres.

To allow for a most even temperature distribution through the whole stack of the assemblies 10 in the carrier 100, first and second raised edges 120, 122 may be provided on the bottom plate 102 along the first and second upstanding walls 104, 106 to elevate the lowest stacked assembly 10 accommodated in the carrier 100. This allows for the lowest stacked assembly 10 to be raised from the bottom plate 102 and avoid full face-to-face contact between the base 16 of the lowest stack assembly 10 and the bottom plate 102, as shown in FIG. 2C, allowing for overall better temperature uniformity during lyophilization throughout the entire stack of the assemblies 10. The first and second raised edges 120, 122 may be continuous or discontinuous and/or located on portions of the bottom plate 102 spaced from the first and second upstanding walls 104, 106 (e.g., provided as lands).

As will be appreciated by those skilled in the art, the assembly 10 may be supported by, and stacked upon, other support components, such as standard lyophilization trays, or used without any supporting components, with the assembly 10 being stacked between walls of a lyophilizer. With any support component, the stack of assemblies 10 should protrude upwardly, beyond the support component, to allow for pressing engagement with an upper shelf of the lyophilizer, without hindrance from the support component (in the same manner as described above in connection with the carrier 100).

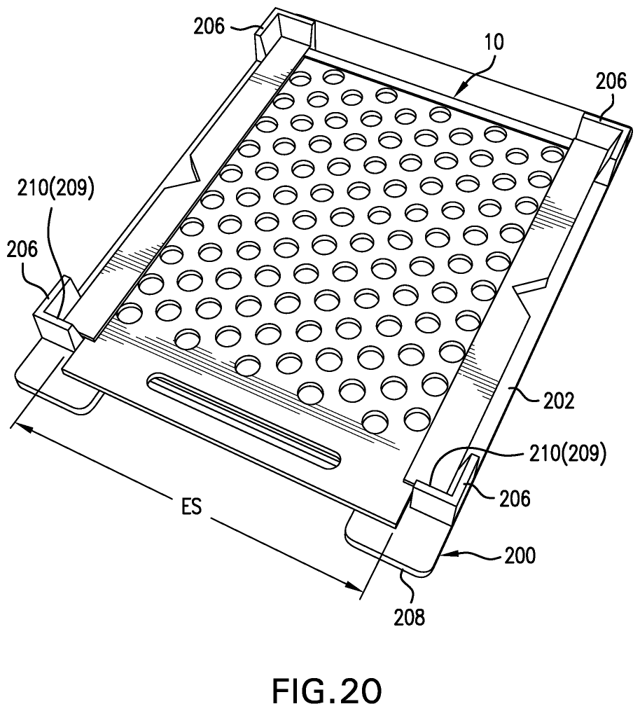

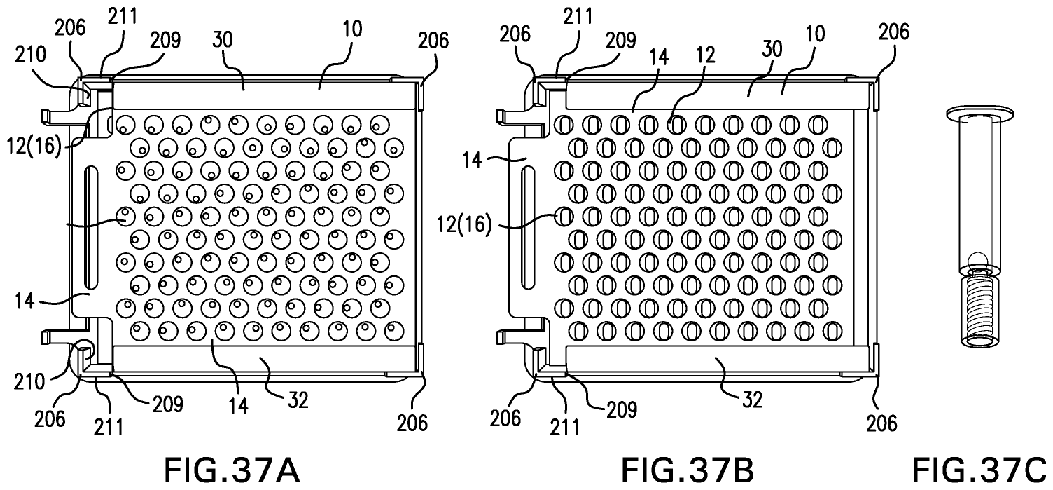

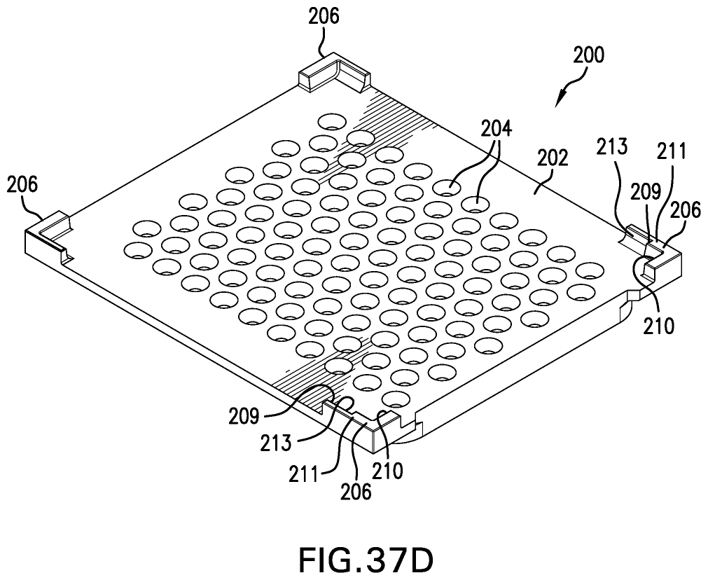

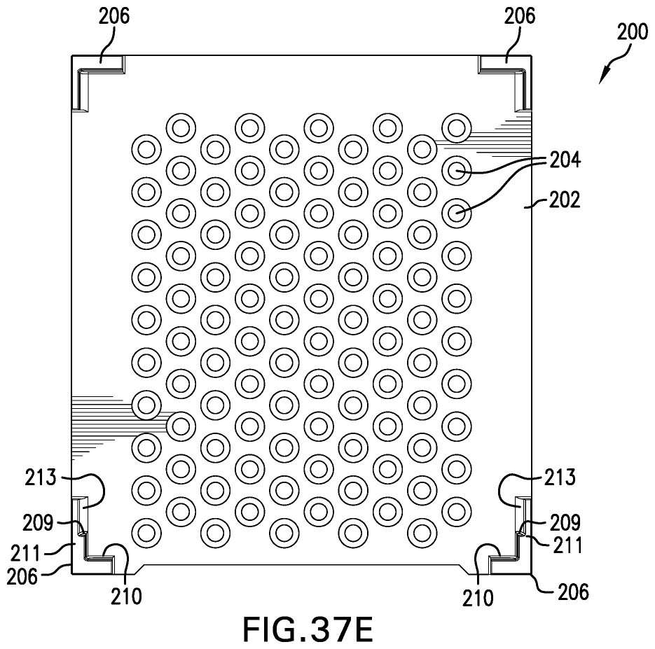

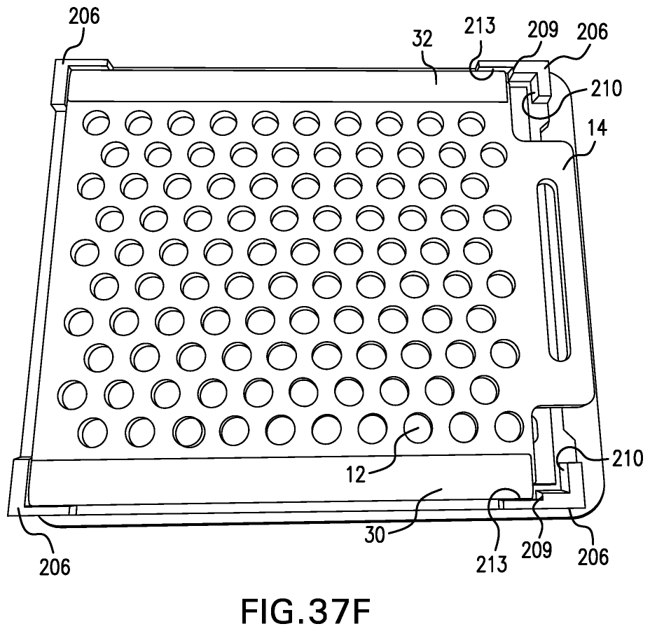

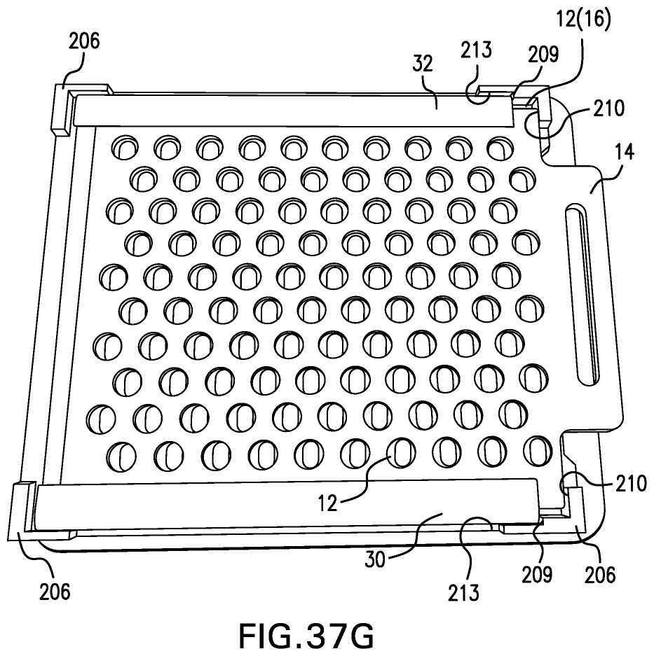

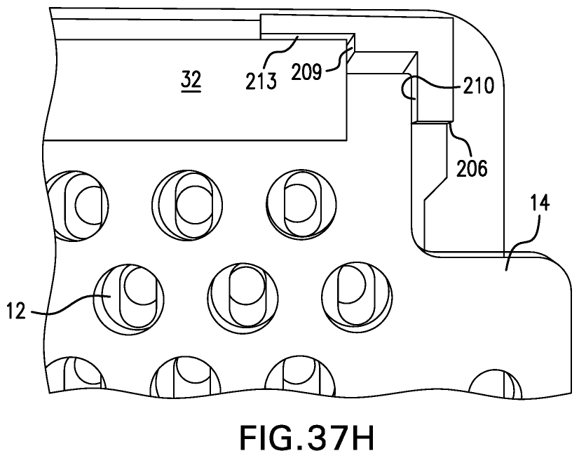

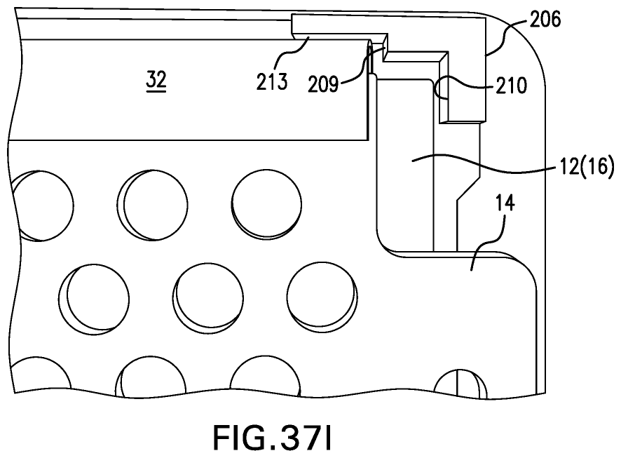

With reference to FIGS. 20-23, a dispensing funnel 200 is shown useable with the assembly 10 as a system for dispensing lyospheres, particularly, after lyophilization. The dispensing funnel 200 includes a support plate 202 having a plurality of fill openings 204 formed therethrough. A plurality of, e.g., four, corner-shaped alignment guides 206 protrude upwardly from the support plate 202. The alignment guides 204 are configured and positioned to receive the assembly 10 and position the assembly 10 atop the support plate 202 in a target position with the openings 18 of the base plate 12 being at least in partial alignment with the fill openings 204 of the support plate 202. The alignment guides 206 inhibit transverse movement of the base plate 12 relative to the support plate 202. In addition, the width X of the insert plate 14 is preferably less than end spacing ES between two of the alignment guides 206 located along a first end 208 of the support plate 202, referenced as the forward alignment guides 206. As shown in FIG. 20, this configuration allows the insert plate 14 to be axially shifted relative to the base plate 12 with inner surfaces 210 of the forward alignment guides 206 defining stop surfaces 209 which interferingly engage with portions of the base plate 12 in inhibiting movement thereof during axial shifting of the insert plate 14. Alternatively, as shown in FIGS. 37A and 37B, the stop surfaces 209 may be formed on rear portions 211 of the forward alignment guides 206. As shown in FIGS. 37D-I, the rear portions 211 may be formed with inner cut-outs 213 extending rearwardly from the stop surfaces 209. As shown in FIGS. 37F, 37H, and 37I, the inner cut-outs 213 may define a width slightly greater than the first and second channels 30, 32 with the stop surfaces 209 being aligned to interferingly engage with portions of the base plate 12 to inhibit movement thereof with axial shifting of the insert plate 14 relative to the base plate 12. With the first and second channels 30, 32 being formed integrally with the base 16, the stop surfaces 209 may be aligned to only interferingly engage with the first and second channels 30, 32 of the base plate 12, although engagement with the base 16 may be also provided. With the first and second channels 30, 32 being provided separately from the base 16 (e.g., as clips), the stop surfaces 209 may be aligned to interferingly engage with both the first and second channels 30, 32, as well as, the base 16 of the base plate 12, to restrict movement of the first channel 30, the second channel 32, and the base 16, with axial shifting of the insert plate 14. In addition, as shown in FIG. 37A, the inner surfaces 210 of the forward alignment guides 206 may be aligned to interferingly engage with the base 16 of the base plate 12, with the stop surfaces 209 being aligned to interferingly engage the first and second channels 30, 32. This configuration allows for the base 16 to have a greater length than the first and second channels 30, 32 and a greater length than portions of the insert plate 14. In addition, this configuration may be also used with the inner cut-outs as shown in FIG. 37G. If the first and second channels 30, 32 are provided as clips, and include rounded corners as described above, portions of the first and second channels 30, 32 may be outwardly bowed away from the insert plate 14 to provide surfaces for engagement against the stop surfaces 209 clear of the insert plate 14.

In addition, as shown in FIGS. 37G and 37H, the inner surfaces 210 of the forward alignment guides 206 may be positioned to act as secondary stop surfaces in limiting the extent of axial shifting of the insert plate 14 relative to the base plate 12. For example, the insert plate 14 may be restricted in axially shifting from the first state (FIG. 37I) to the second state (FIGS. 37G and 37H), as described above. Here, the width X of the insert plate 14 may be formed greater than the end spacing ES between the forward alignment guides 206, so that axial shifting of the insert plate 14 relative to the base plate 12 is limited by the forward alignment guides.

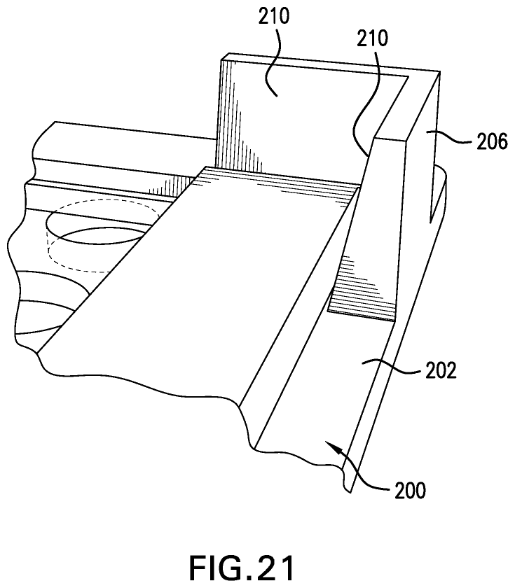

One or more of the inner surfaces 210 of the alignment guides 206 may be tapered to guide the assembly 10 to the target position atop the support plate 202, as shown in FIG. 21.

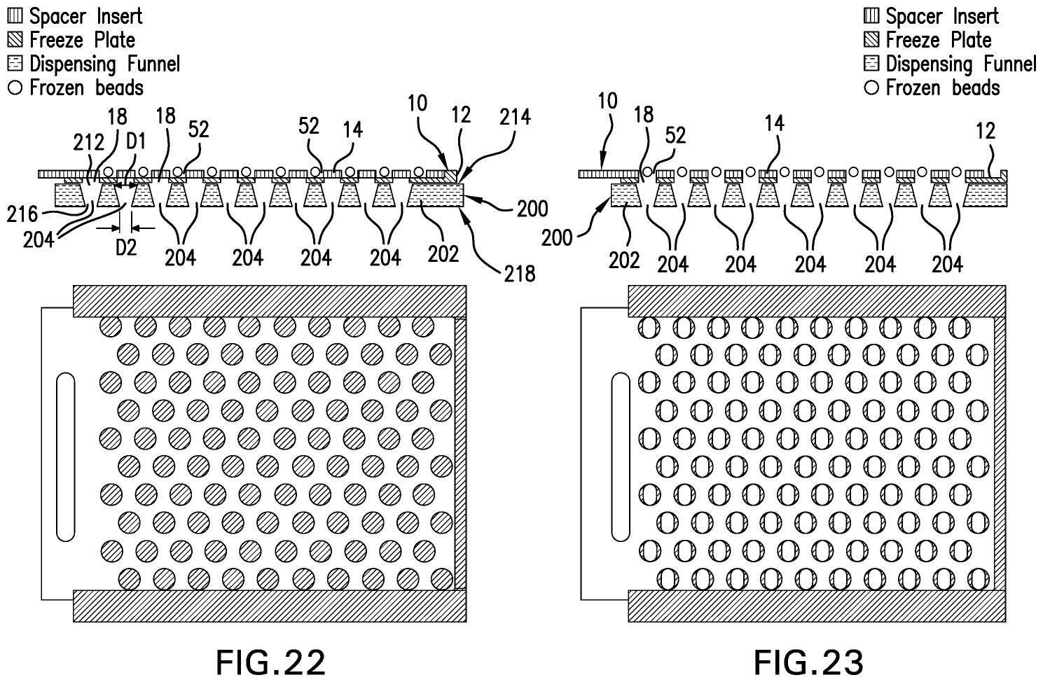

The fill openings 204 may be formed with constant diameters along their respective lengths. Alternatively, as shown in FIGS. 22 and 23, the fill openings 204 may each have a non-constant diameter along their respective length with a top opening 212, in a top face 214 of the support plate 202, having a first diameter D1, and a bottom opening 216, in a bottom face 218 of the support plate, having a second diameter D2 which is less than the first diameter D1. The top opening 212 may define a larger area than the bottom opening 216. The top openings 212 are at least in partial alignment with the openings 18 with the assembly 10 in the target position on the support plate 202. The bottom openings 216 allow the lyospheres to be dispensed therefrom. The fill openings 204 may be treated or coated to minimize static electrical charge therein which may affect descent of the lyospheres.

FIG. 22 shows the assembly 10 atop the dispensing funnel 200 with the assembly 10 in the first state. FIG. 23 shows the assembly 10 atop the dispensing funnel 200 with the assembly 10 in the second state. As shown in FIG. 23, the lyospheres are free to fall from the apertures 52, through the openings 18 and the fill openings 204, into a contained area or containers, each having an opening aligned with one of the fill openings 204. The fill openings 204, particularly in the bottom face 218 around the bottom openings 216, may be provided with notches, slots, etc. to complementarily mount onto target containers. In addition, the bottom face 218 may be contoured to shape-matingly engage a tub, tray, or nest which contains the target containers. These arrangements allow for proper relative positioning between the containers and the dispensing funnel 200.

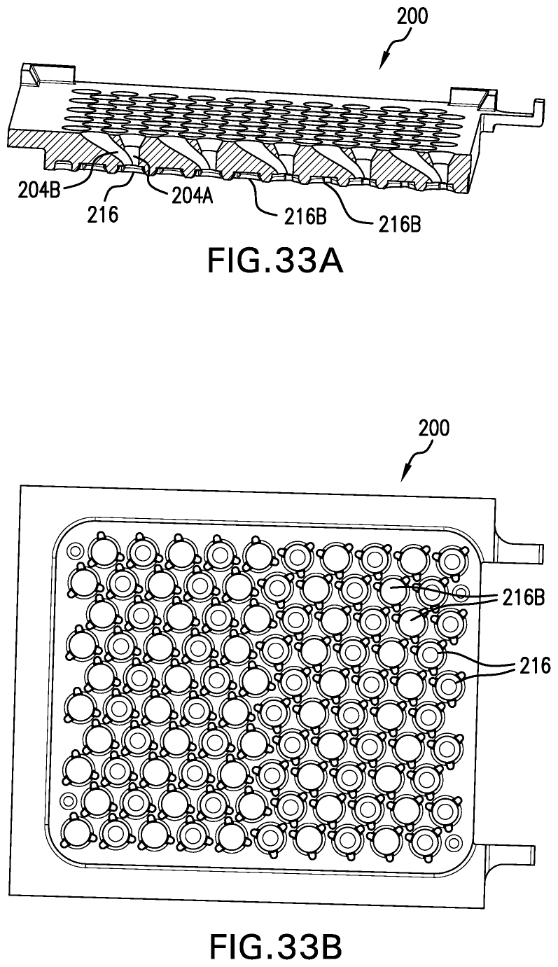

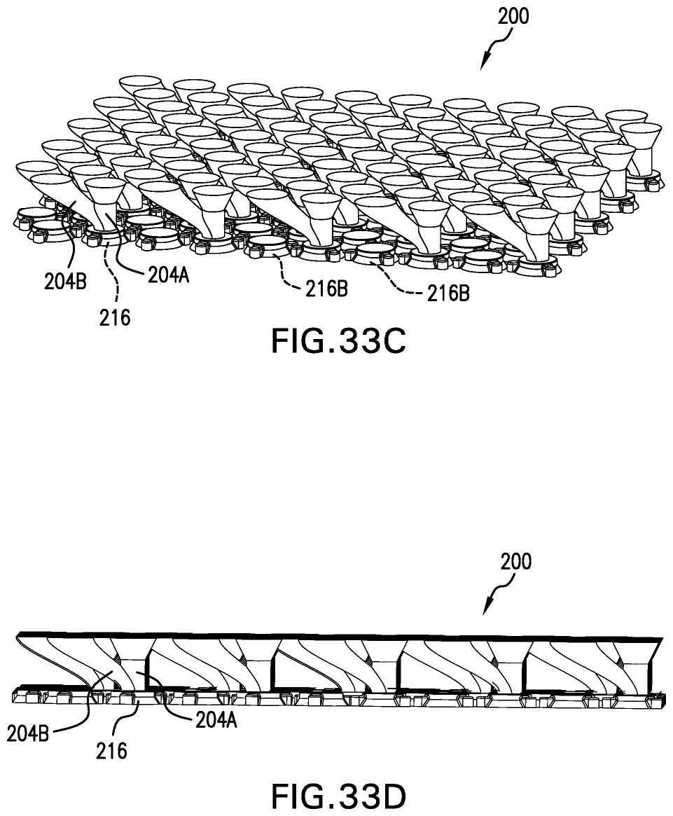

With reference to FIGS. 33A-33D, the fill openings 204 may be combined so that a plurality of lyospheres is dispensed into individual target containers. FIGS. 33A-33D show two of the fill openings 204 being combined with primary fill openings 204A being generally vertical and secondary fill openings 204B being transversely arranged to merge with the primary fill openings 204A. It is preferred that the secondary fill openings 204B define a sufficiently continuous downward path to limit lyospheres from losing momentum during travel through the secondary fill openings 204B. It is also preferred that the secondary fill openings 204B merge with the primary fill openings 204A adjacent the bottom openings 216 of the primary fill openings 204A. Blind (closed) depressions 216B may be provided to align with the secondary fill openings 204B to provide an indication of alignment.

As will be appreciated by those skilled in the art, various quantities of the fill openings 204 may be combined to allow for different quantities of lyospheres to be delivered to common containers. It is noted that even distribution requires even division of available lyospheres. For example, with an array of 100 lyospheres, even distribution of multiples units is achievable with the dispensing of 1, 2, 4, 5, 10, 20, 25, or 50 units per container. Other combinations may be achieved by altering the quantity of available lyospheres. For example, the base plate 12 may be provided with only a partial quantity of the lyospheres, e.g., 90 units, rather than the full amount, e.g., 100 units. This allows for different multiples of units per container to be achieved, such as 1, 2, 3, 5, 6, 9, 10, 15, 18, 30, or 45 units per container. The array may be increased to allow for greater quantities as well, such as 120 lyospheres.

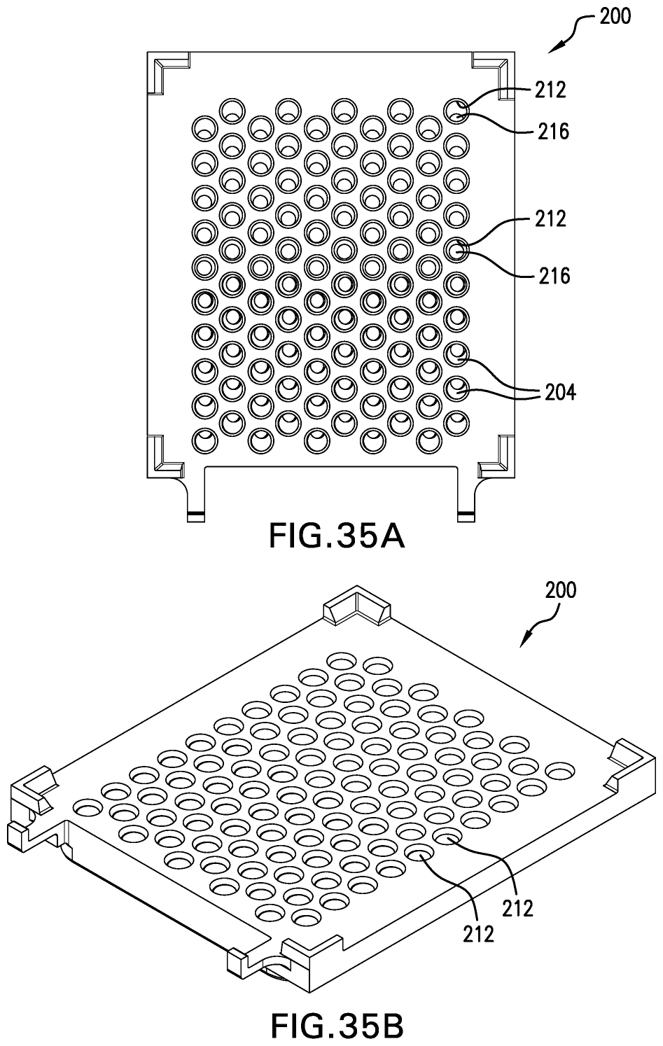

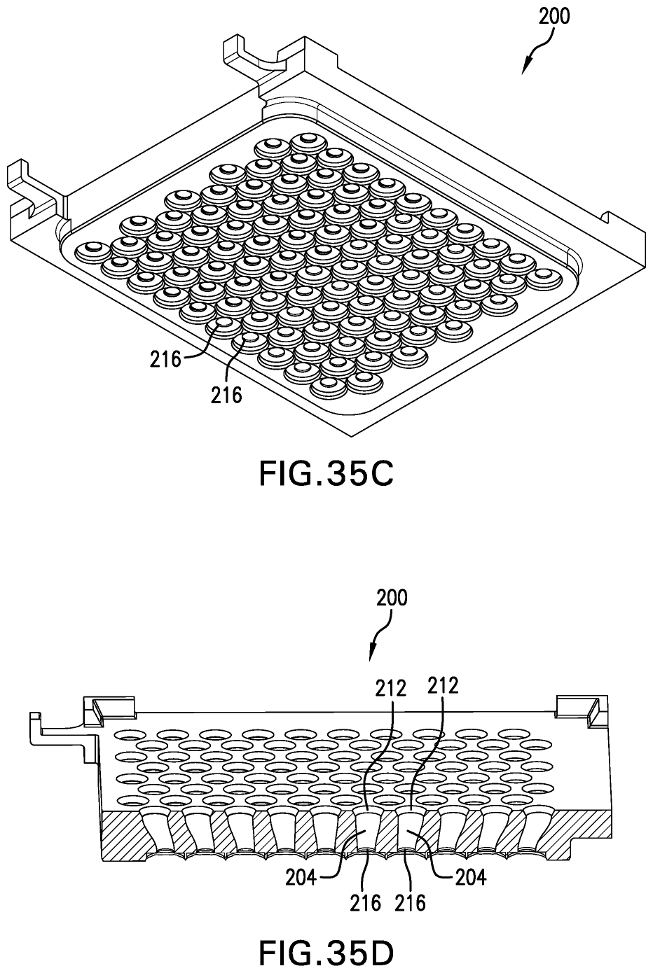

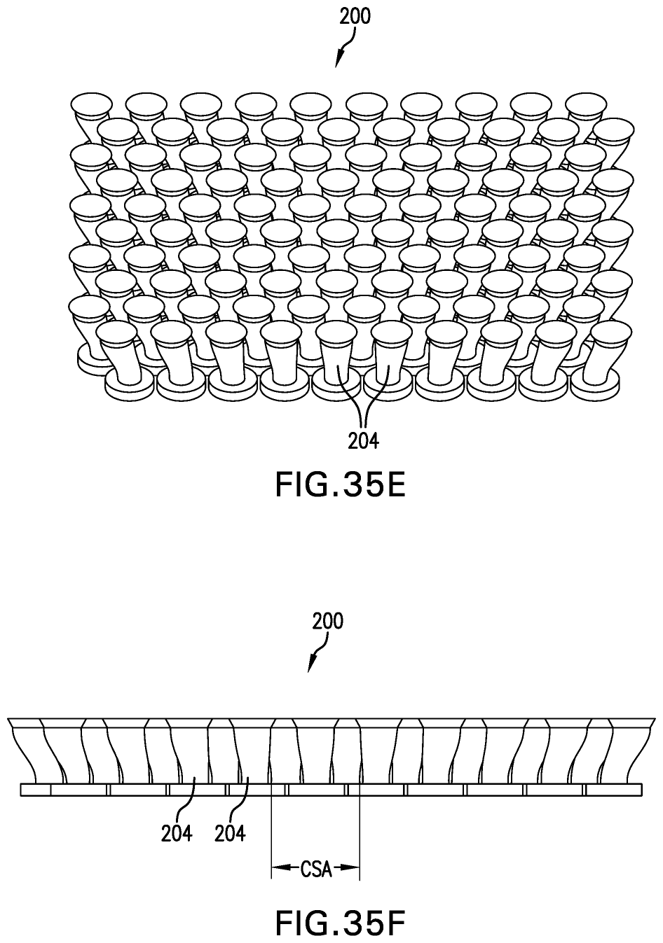

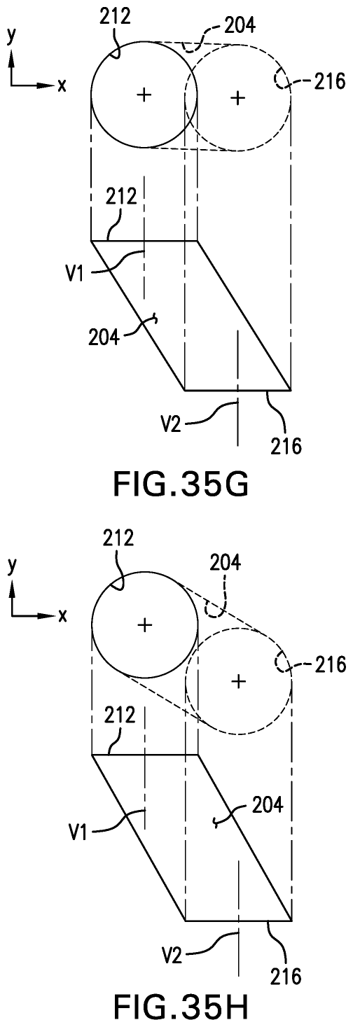

With reference to FIGS. 35A-35H, the fill openings 204 may be differently configured to accommodate different arrays of containers for dispensing. This allows for provision of different dispensing funnels 200 which may be accommodated in the same equipment, i.e., having equivalent outer dimensioning, but having altered arrays of the bottom openings 216 to accommodate different arrays of containers (more or less compacted). For example, FIGS. 35A-35F shown an arrangement of the fill openings 204 that may be used for filling a tub of syringe barrels, which due to relatively small diameters, have relatively high packing density. Here, the fill openings 204 of a central band-shaped sub-array CSA may be each provided with a generally vertical alignment (with the top openings 212 and the bottom openings 216 being generally vertically aligned) with bands of the fill openings 204 being increasingly offset in radially outwardly directions from the central sub-array CSA (with the bottom openings 216 being increasingly out of vertical alignment with the top openings 212). The provides a relative dense array of the bottom openings 216 with the top openings 212 defining a more spacious array.

FIGS. 35A-35F show the offset changing in one direction relative to the dispensing funnel 200. This is shown more clearly in FIG. 35G where vertical axis V1, which perpendicularly intersects the center of the top opening 212, is offset from vertical axis V2, which perpendicularly intersects the center of the bottom opening 216, along one coordinate axis X. As shown in FIG. 35H, the offset between the vertical axes V1, V2 may change along two coordinate axes X, Y, allowing for rings of the fill openings 204 with altering offset.

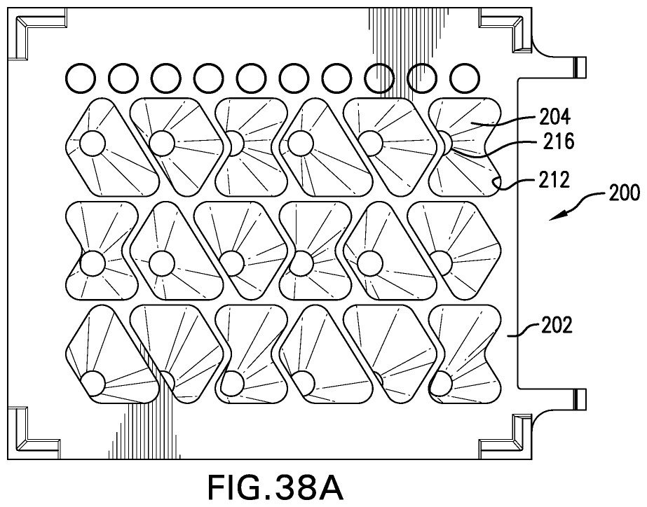

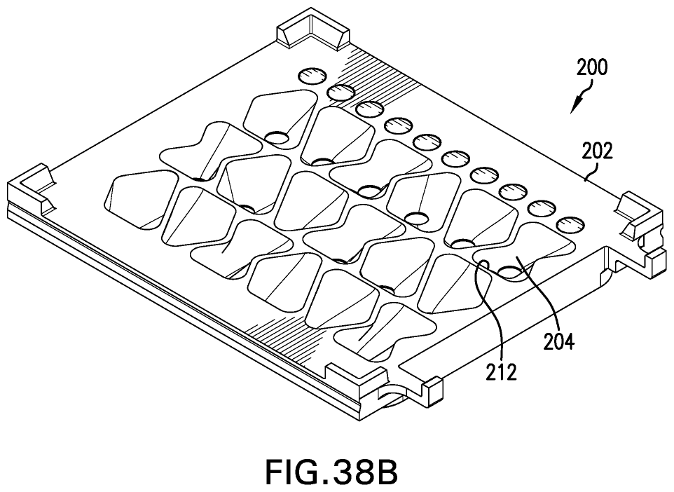

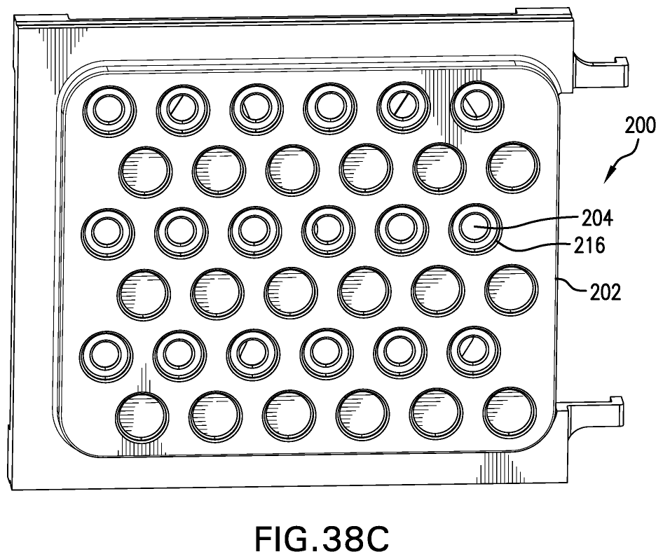

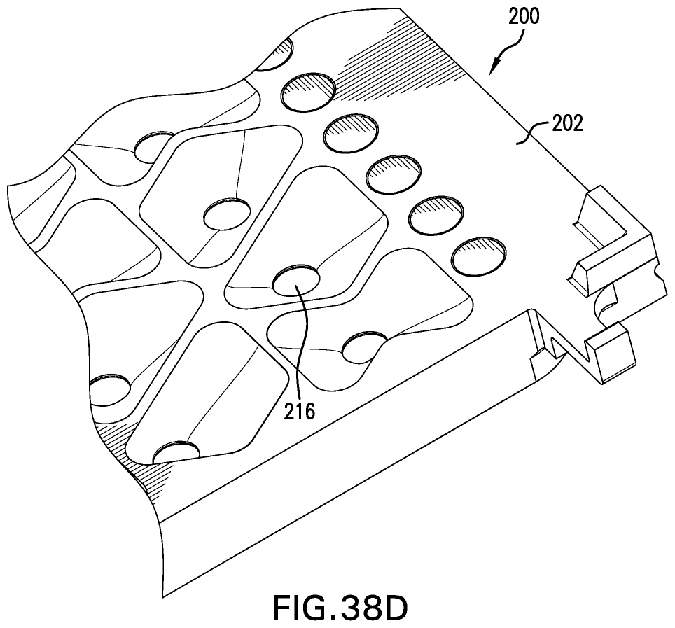

As shown in FIGS. 38A-38D, the top openings 212 may be configured to align with a plurality of the openings 18. This allows for a plurality of lyospheres to be dispensed through common fill openings 204. For example, each of the top openings 212 may be formed to align with five of the openings 18 to allow for 90 of the lyospheres to be evenly dispensed into 18 containers. Other configurations are possible.

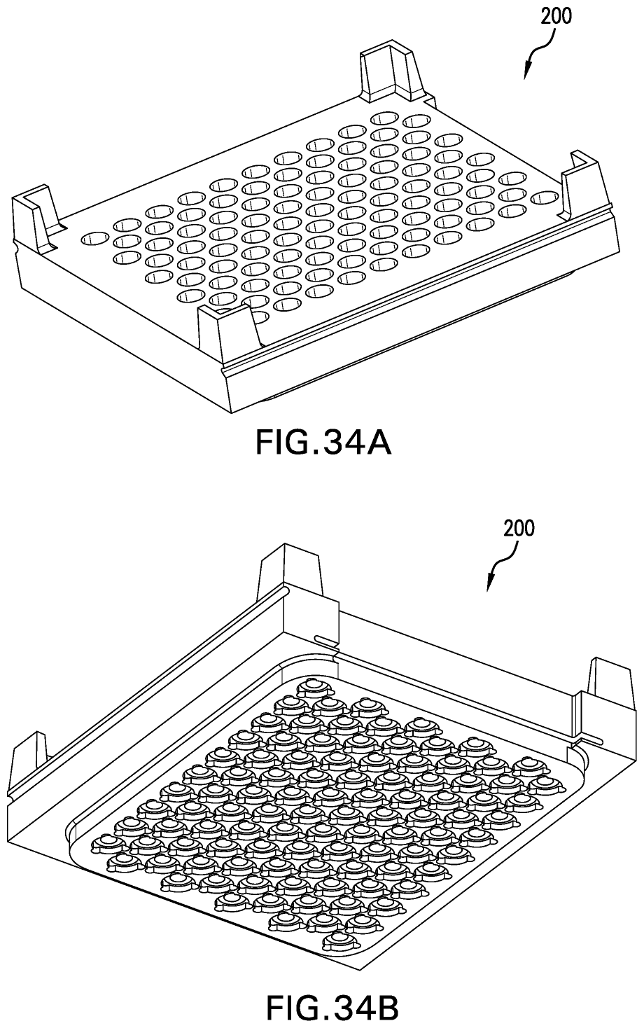

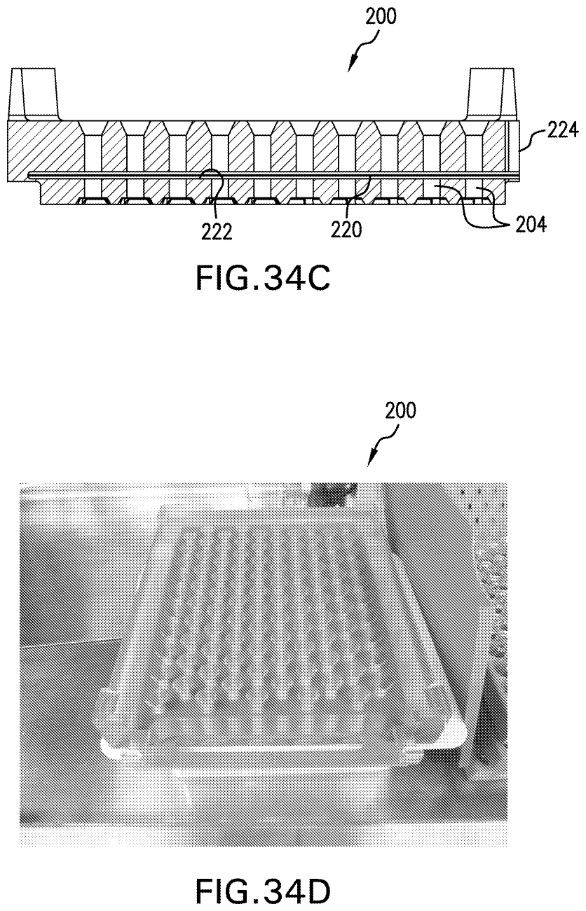

As shown in FIGS. 34A-34D, the dispensing funnel 200 may be further modified to include a removable collection plate 220 insertable in slot 222 to intersect the fill openings 204, forming a physical barrier thereacross. The slot 222 may be formed in the first end 208 of the body 202. The collection plate 220 allows for lyospheres to be dispensed and collected in the fill openings 204. With a target number of the lyospheres having been dispensed, the collection plate 220 may be removed, thereby opening the fill openings 204 to allow for dispensing into target containers located below the dispensing funnel.

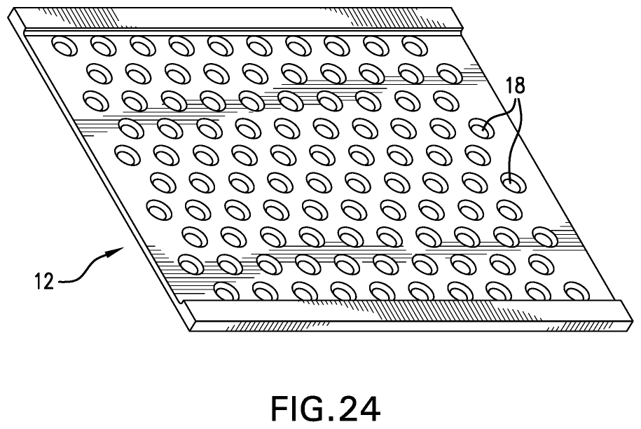

With reference to FIG. 24, in an alternative embodiment, the assembly 10 may be provided with only the base plate 12. Here, the openings 18 are provided as closed wells, each forming a depression for the receiving of droplets. This allows for formation of the lyospheres, with dispensing performed by a method different from gravity drop through an opening in the base plate. Once formed, the lyospheres may be collected and dispensed by a pick-and-place machine or other construct which allows for collection of the lyospheres from the wells 18.

### EXAMPLES

The examples in this section are offered by way of illustration, and not by way of limitation.

### Example 1. Preparing Lyospheres Using Assemblies Including Aluminum Base Plates

Assemblies including aluminum base plates with aluminum thermal conduction spacers and plastic insert plates were placed on top of aluminum bonded finned heat sinks, which sat in a tray packed with dry ice. The whole structure (assemblies, heat sinks, tray and dry ice) was blast frozen at −115° C. Immediately after removal from the blast freezer, 50 microliter aliquots of Formulation I were pipetted into each of the apertures in the insert plate to be supported by the base plate, and quickly froze into frozen droplets on the cold base plate. Assemblies that each had 100 frozen droplets were stacked on top of each other in a −70° C. freezer. The bottom-most base plate in the stack did not have frozen droplets (level 1), while the 8 levels above level 1 in the stack (levels 2 through 9, counting vertically upwards) had frozen droplets on their base plates. The stack also had a top aluminum base plate that rested on top of the thermal conduction spacers above level 9. Frozen droplets remained in the stack at −70° C. overnight, and lyophilization was conducted the next day. The 9-level stack was placed in an SP Lyostar 2 lyophilizer on the lowest lyophilizer shelf, which had been pre-cooled at −55° C. The lyophilizer shelves were moved together (using the lyophilizer stoppering capability) so that the stack was compressed between two lyophilizer shelves, so that the top and the bottom aluminum base plates of the stack were in essentially full contact with a temperature controlled lyophilizer shelf. Table 1 shows the lyophilization drying program.

TABLE 1
Lyophilization drying program

| Step | 1 | 2 | 3 | 4 | 5 | 6 |
|---|---|---|---|---|---|---|
| Shelf Setpoint, ° C. | −43 | −27 | 0 | 36 | 31 | 2 |
| Ramp Rate, ° C./min | 2 | 2 | 0.1 | 0.2 | 1.0 | 0 |
| Hold Time, min | 54 | 1 | 1 | 1 | 431 | hold |
| Vacuum, mTorr | 50 | 50 | 50 | 50 | 50 | 50 |

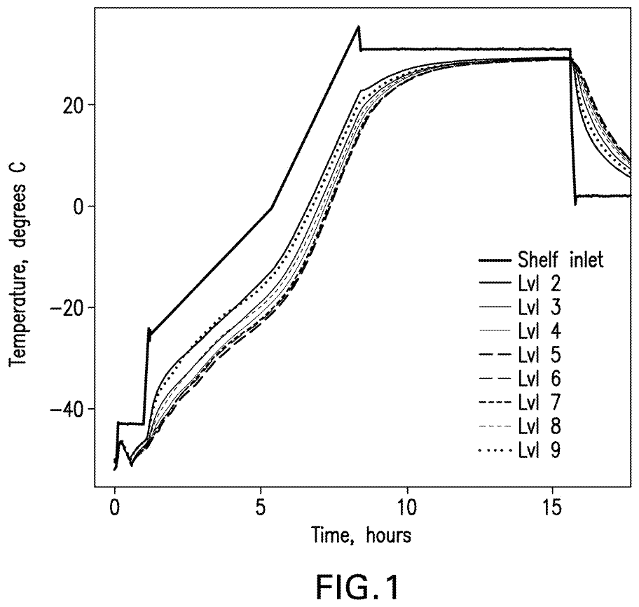

Drying was complete, and the ramp down to the 2° C. hold started at about 15.6 hours. Temperature of each level base plate was measured by thin wire thermocouples taped firmly to the base plates with cleanroom tape. The temperatures of the aluminum base plates tracked each other closely during the lyophilization process and converged during secondary drying at about 29° C. (FIG. 1). The dried lyospheres had good visual appearance.

### Example 2. The Temperature of a Base Plate in Full Contact With a Lyophilizer Shelf Can be Significantly Different From the Temperature of Base Plates That are not in Full Contact With the Lyophilizer Shelf

This experiment compared the performance of two stacks of assemblies with slightly different configurations during lyophilization. Specifically, in the first configuration (FIG. 2A), the stack had 8 levels of assemblies. The base plate of the lowest assembly was in full contact with the lyophilizer shelf, and all 8 assemblies had frozen droplets for lyophilization. In the second configuration (FIG. 2B), the stack had 9 levels of assemblies, but only the top 8 assemblies had frozen droplets on their base plates. FIG. 2C illustrate an alternative to the second configuration, wherein the lowest level assembly that had no frozen droplets was replaced with thermal conduction side spacers. Plastic insert plates (not shown in FIGS. 2A-2C) were overlaying on each aluminum base plate to keep frozen droplets separate. In all configurations, an aluminum base plate was placed on the top of the thermal conduction side spacers on the topmost level.

The temperature of each base plate that had frozen droplets (in FIG. 2A, levels 1-8; in FIG. 2B, levels 2-9) was monitored during lyophilization. The stacks were compressed between lyophilizer shelves during the lyophilization (using the lyophilizer stoppering capability) so that the top and the bottom aluminum base plates were in essentially full contact with a temperature controlled lyophilizer shelf.

Fifty microliter aliquots were manually pipetted onto the ultracold aluminum base plates to form the frozen droplets. One droplet was formed inside each aperture of the plastic insert plate (100 frozen droplets per plate). Formulation II was used for the configuration in FIG. 2A. Formulation III was used for levels 2-5 (counting from bottom up) of the configuration in FIG. 2B, and Formulation IV was used for levels 6-9 of the configuration in FIG. 2B.

Lyophilization was performed in an SP Lyostar2 lyophilizer. Thin wire thermocouples were securely taped onto each of the base plates that had frozen droplets. The lyophilization drying programs are listed below in Tables 2 and 3.

TABLE 2
Lyophilization drying program for the configuration in FIG. 2A

| Step | 1 | 2 | 3 | 4 |
|---|---|---|---|---|
| Shelf Setpoint, ° C. | −50 | 20 | 30 | 0 |
| Ramp Rate, ° C./min | 0 | 1 | 1 | 0 |
| Hold Time, min | 60 | 900 | 360 | hold |
| Vacuum, mTorr | 50 | 50 | 50 | 50 |

TABLE 3
Lyophilization drying program for the configuration in FIG. 2B

| Step | 1 | 2 | 3 |
|---|---|---|---|
| Shelf Setpoint, ° C. | −45 | 30 | 30 |
| Ramp Rate, ° C./min | 2 | 2 | 0 |
| Hold Time, min | 240 | 780 | hold |
| Vacuum, mTorr | 50 | 50 | 50 |

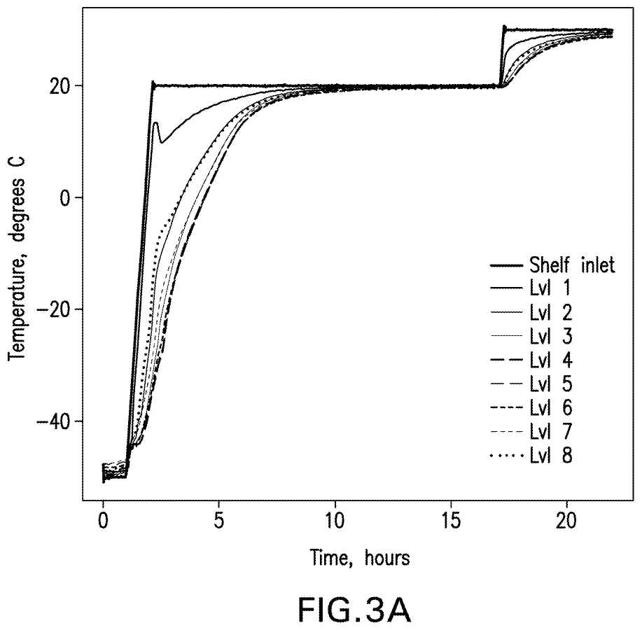

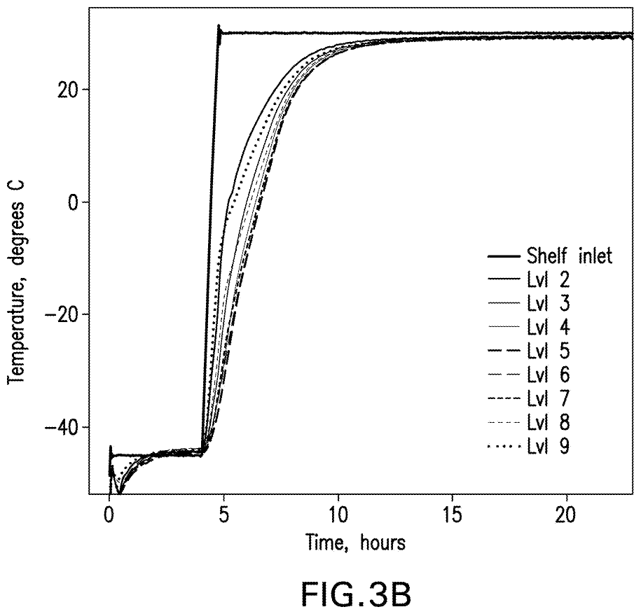

It is a surprising discovery that the lowest base plate that was in essentially full contact with the lyophilizer shelf had temperature kinetics that significantly departed from the other base plates within the stack (FIG. 3A). The temperature difference was large enough that when the lyophilizer program stopped ramping the shelf temperature and held it constant at target (20° C.), heat drawn from the lowest base plate to warm the base plates above resulted in a prominent dip in the thermocouple trace for the lowest base plate (shown in FIG. 3A). When the temperatures and thermal kinetics are significantly different among the base plate levels, it is difficult to develop and implement lyophilization cycles and to achieve the desired product consistency. As a solution, improved stack configurations were designed to improve uniformity of base plate temperatures during the lyophilization process. By not lyophilizing frozen droplets on the lowest base plate that directly rested upon the lyophilizer shelf (FIG. 2B) or by raising the lowest base plate that had frozen droplets using thermal conduction side spacers (FIG. 2C), all frozen droplets in the entire stack experience a more consistent thermal profile throughout lyophilization. FIG. 3B shows that, in the stack configuration of FIG. 2B, temperature differences among base plates were decreased, and temperature kinetics of different base plates closely resemble each other.

### Example 3. Aluminum Base Plates are Superior to Stainless Steel Base Plates in Maintaining Temperature Uniformity Among Assemblies Within a Stack

This experiment compared two different base plate materials, aluminum and stainless steel, for their ability to maintain temperature uniformity among assembly levels within a stack. A 9-level stainless steel assembly stack (comprising stainless steel base plates, stainless steel thermal conduction spacers, and plastic insert plates) had no frozen droplets on the lowest level (level 1) and frozen droplets of Formulation V on levels 2-5 and Formulation IV on levels 6-9. The stack also had a stainless-steel plate that rested on top of the thermal conduction spacers above level 9, the topmost assembly. The 9-level stack was placed in an SP Lyostar 2 lyophilizer on the lowest lyophilizer shelf, which had been pre-cooled at −50° C. On the shelf above (the middle shelf) was placed a similar stack comprised of aluminum base plates, aluminum thermal conduction spacers, plastic insert plates, and an aluminum top plate, but without frozen droplets. The lyophilizer shelves were moved together (using the lyophilizer stoppering capability) so that both stacks were compressed between two lyophilizer shelves, and the top plate and the bottom plate of both stacks were in full contact with a temperature controlled lyophilizer shelf The lyophilization drying program is shown in Table 4.

TABLE 4
Lyophilization drying program

| Step | 1 | 2 | 3 | 4 |
|---|---|---|---|---|
| Shelf Setpoint, ° C. | −43 | −15 | 30 | 30 |
| Ramp Rate, ° C./min | 2 | 2 | 0.2 | 0 |
| Hold Time, min | 54 | 1 | 600 | Hold |
| Vacuum, mTorr | 50 | 50 | 50 | 50 |

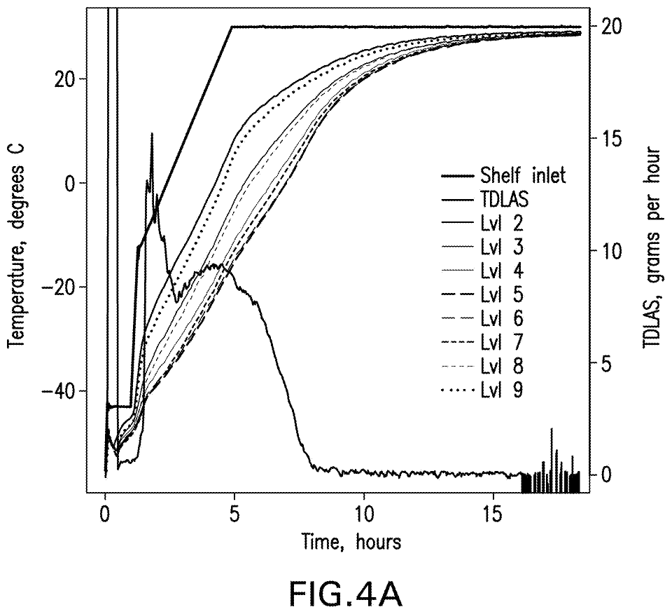

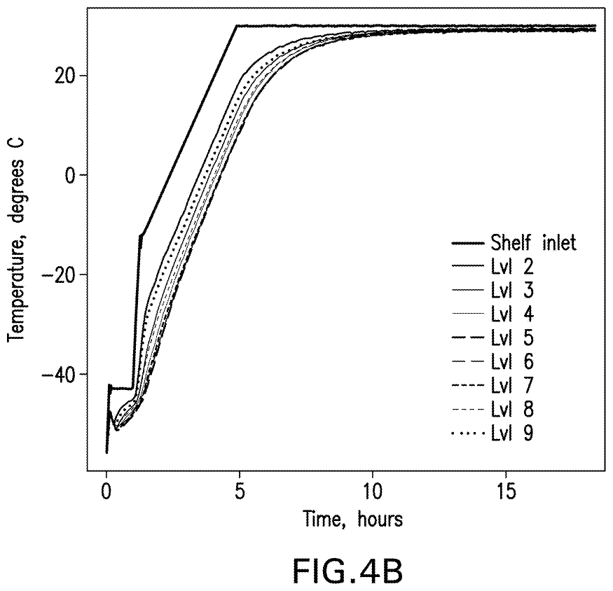

Primary drying appeared complete (based on TDLAS, tunable diode laser absorption spectroscopy) under 10 hours. Temperature of each base plate was measured by thin wire thermocouples taped firmly to the base plates with cleanroom tape. The temperatures of the stainless-steel base plates tracked each other closely during the lyophilization process and converged during secondary drying (FIG. 4A). The dried lyospheres of both formulations had good visual appearance. The temperatures of the base plates in the aluminum stack (FIG. 4B) tracked more closely than those in the stainless-steel stack. Aluminum appears to have an advantage over stainless steel in maintaining temperature uniformity among assemblies within a stack. However, it is recognized that stainless steel is widely used for product contact surfaces in GMP manufacture of biologics and pharmaceuticals, and is suitable for use in the methods described herein.

### Example 4. Dispensing Lyospheres From an Assembly into Containers

The base plates in the assemblies exemplified in Examples 1-3 can be solid surface plates or plates with an array of openings. For the base plates with an array of openings, solid portions of the plate are located between and surrounding the openings. The insert plate overlaying the base plate can be axially moved relative to the base plate from a first state to a second state. In the first state, the apertures in the insert plate align with the solid portions of the base plate but with no overlap with the openings of the base plate. In the second state, the apertures are at least partially aligned with the openings so as to at least partially overlap the openings. In the steps of dispensing droplets on the base plate, freezing the droplets, or drying the droplets in a lyophilizer, the insert plate is in the first state relative to the base plate. After lyophilization, the dried lyospheres can be dispensed into containers by shifting the insert plate from the first state to the second state. An exemplary support plate that facilitates the dispensing of lyospheres is illustrated below.

A support plate with an array of funnels was designed to firmly seat on vials nested in a particular 100-vial nest (Schott AdaptiQ vial nest for 2R vials). While the insert plate was in the first state relative to the base plate, a glass bead (approximately 5 mm diameter, in a similar size range as some lyospheres) was placed in each of the 100 apertures in the insert plate to be supported by the solid portions of the base plate. The assembly with the beads was placed on top of the support plate with the array of funnels, which was seated on the vials in the vial nest. A small shift (about 1 centimeter displacement) of the insert plate from the first state to the second state resulted in all 100 beads falling through the funnels essentially simultaneously, one bead each directly into their respective vials below.

The process can be repeated with additional assemblies to achieve the number and types of lyospheres desired in each vial, syringe, or other pharmaceutically acceptable containers in an array format. For example, multiple lyospheres of different pharmaceutical compositions can be dispensed into one container to produce a combination drug product (e.g., multivalent vaccines, combination therapeutics, etc.).

### Example 5. Preparing and Dispensing Lyospheres of Multiple Formulations into the Same Containers

Two liquid formulations were prepared. One of the formulations comprised a red dye, allowing the two formulations to be easily distinguished upon simple visual inspection. Assemblies were placed on top of a heat sink and chilled to a low temperature. The base plates were not physically attached to the heat sink. While the insert plates are in the first state relative to the base plates, 50 microliter droplets of the formulations were dispensed on the base plates in an array format, and the droplets froze on the base plates. The red colored formulation was frozen on the base plate of one of the assemblies, and the other formulation was frozen on the base plate of the other seven assemblies.

The process of liquid dispensing and freezing on the base plates was repeated several times, and the base plates with frozen droplets were stacked one above another with a thermally conductive path formed between the assemblies, and the stack of eight assemblies (held within a carrier) were placed in a lyophilizer and lyophilized. The frozen droplets were lyophilized on the same base plates on which they were frozen, in an array format, with assemblies stacked one above another. The stoppering function of the lyophilizer was used to move the lyophilizer shelves together, so that the stack of assemblies was compressed between a lyophilizer shelf from below and a lyophilizer shelf from above.

The lyophilization drying program used for this example is shown in Table 5.

TABLE 5
Lyophilization drying program

| Step | 1 | 2 | 3 | 4 |
|---|---|---|---|---|
| Shelf Setpoint, ° C. | −50 | −40 | −20 | +20 |
| Ramp Rate, ° C./min | (blank) | 0.1 | 0.1 | 0.1 |
| Hold Time, min | 120 | 120 | 2880 | 360 |
| Vacuum, mTorr | 30 | 30 | 30 | 30 |

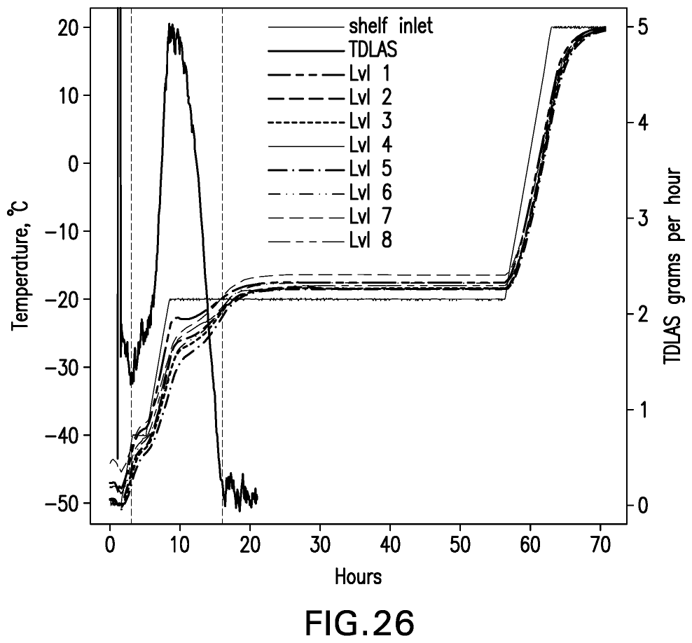

The hold time at −20° C. was intentionally much longer than needed for primary drying, as an experiment to determine how long it would take to complete primary drying of these particular formulations at −20° C. During lyophilization, each of the eight stacked assemblies had a thermocouple firmly taped to one side of the base plate, and thermocouples were also taped to both sides of the carrier, to obtain information about temperature uniformity. FIG. 26 shows thermocouple temperatures during lyophilization, the shelf inlet temperature, and TDLAS data showing mass of removed water, all as a function of time. As seen in FIG. 26, primary drying at −20° C. was complete within significantly less than 20 hours. It is recognized that the lyophilization program can be further optimized.

After drying, the carrier with the stack of assemblies with lyospheres was removed from the lyophilizer into a glovebox with a predominantly dry nitrogen atmosphere. Lyospheres in array format were dispensed into glass vials in an array format by placing a dispensing funnel on top of an array of one hundred glass vials, then placing an assembly with lyospheres on top of the dispensing funnel and axially shifting the insert plate relative to the base plate to the second state. This resulted in all one hundred lyospheres falling directly into one hundred glass vials, essentially simultaneously. This was repeated for all eight assemblies, resulting in eight lyospheres per glass vial (confirmed by counting beads in each individual vial). Each of the glass vials had seven white lyospheres and one red lyosphere, demonstrating the ability of this process to prepare final containers with multiple different types of lyospheres. The lyospheres dispensing process took less than two minutes, and all 800 lyospheres were properly directed to the intended vial. The lyospheres had good visual appearance.

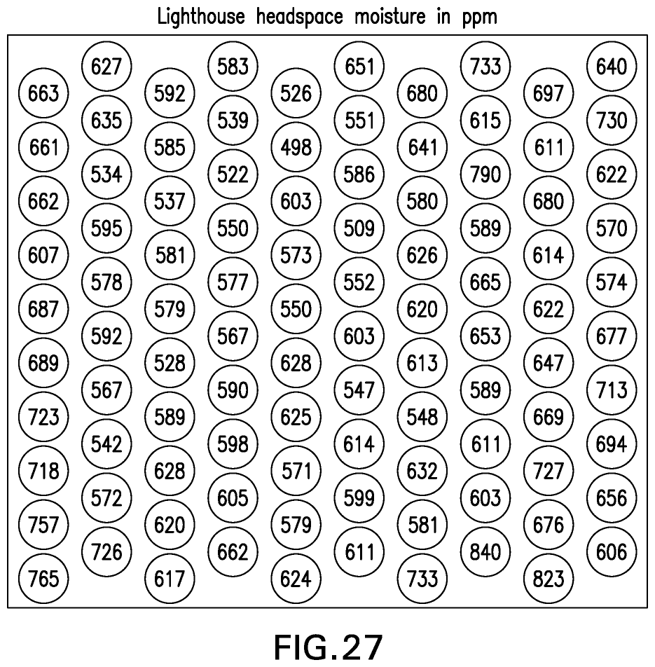

To understand the degree of uniformity in moisture content for this particular batch, lyospheres (initially collected as eight lyospheres per vial into “2R” vials) were further placed within larger glass vials for Lighthouse headspace moisture analysis. Large Lighthouse vials were stoppered within the glovebox with a predominantly dry nitrogen atmosphere. Each of the one hundred vials was analyzed using the Lighthouse instrument to measure the moisture in the headspace of the stoppered large vials. This measurement was done approximately one day after stoppering the vials, with storage and measurement at room temperature. FIG. 27 shows the water moisture level in the headspace of each vial in parts per million (ppm), with the vial positions shown as they were in the array when they were filled with lyospheres. (For comparison, the average value for an empty vial stoppered in the glovebox when measured by Lighthouse on the same occasion was about 4500 ppm, demonstrating that when lyospheres were sealed in vials inside the glovebox, they still absorbed some moisture from the glovebox atmosphere.) These results demonstrate that the moisture content across the 100 vials of this particular batch is within reasonably consistent range, indicating that preparing lyospheres using the assemblies and methods described in this disclosure produces good quality lyospheres with consistent moisture content.

### Example 6. Preparing and Dispensing Lyospheres of Multiple Formulations Using a Combiner Dispensing Funnel or an Accumulator Dispensing Funnel

Two different dispensing funnels were used in Example 6, both of which differ from the dispensing funnel used in Example 5. One of the dispensing funnels used in Example 6 had the property of directing an array of lyobeads into an array of one half as many vials (or in the general case, an array of lyobeads into an array of fewer final containers, in this case targeting the same number of lyobeads per final container). In Example 6, this was 100 lyobeads being directed into 50 vials, each vial to receive 2 lyobeads. This first dispensing funnel is referred as a combiner dispensing funnel (FIGS. 33A-33D). The other dispensing funnel used in Example 6 had the property of having a storage or accumulation space between the top surface and the bottom surface, still maintaining an array, into which lyobeads from more than one assembly can be accumulated and held for a time before dispensing together into vials or other final containers. This second dispensing funnel is referred as an accumulator dispensing funnel (FIGS. 34A-34D).

Two liquid formulations were prepared. One of the formulations comprised a red dye, while the other was a non-dye containing formulation (thus white in color), allowing the two formulations to be easily distinguished upon simple visual inspection. Assemblies were pre-cooled in a −70° C. freezer overnight prior to use for freezing lyobeads. Heat sinks packed in a tray of dry ice were blast frozen at −115° C., following which pre-cooled assemblies were removed from the −70° C. freezer and placed on top of the chilled heat sinks. Base plates were not physically attached to the heat sink. While insert plates are in the first state relative to the base plates, 50 microliter droplets of the formulations were dispensed on the base plates in an array format, and the droplets froze on the base plates. Assemblies with frozen beads in an array were placed back in the −70° C. freezer until the start of the lyophilization cycle. Both of these formulations (with and without red dye) have a low Tg′ (glass transition temperature for the frozen liquid) of about −39° C. To demonstrate useful aspects of dispensing lyobeads using a combiner dispensing layer or an accumulator dispensing layer, on some of the assembly base plates the two formulations of red and white beads were frozen on the same base plate in alternating rows.

The base plates with frozen droplets were stacked one above another with a thermally conductive path formed between the assemblies, and the stack of eight assemblies (held within a carrier) was placed in a lyophilizer. The frozen droplets were lyophilized on the same base plates on which they were frozen, in an array format, with assemblies stacked one above another. The stoppering function of the lyophilizer was used to move the lyophilizer shelves together, so that the stack of assemblies was compressed between a lyophilizer shelf from below and a lyophilizer shelf from above.

The lyophilization drying program used for this example is shown in Table 6.

TABLE 6
Lyophilization drying program

| Step | 1 | 2 | 3 | 4 |
|---|---|---|---|---|
| Shelf Setpoint, ° C. | −50 | −22 | +30 | +15 |
| Ramp Rate, ° C./min | (initial) | 0.3 | 0.2 | 1 |
| Hold Time, min | (initial) | 2100 | 480 | (final hold) |
| Vacuum, mTorr | 30 | 30 | 30 | 30 |

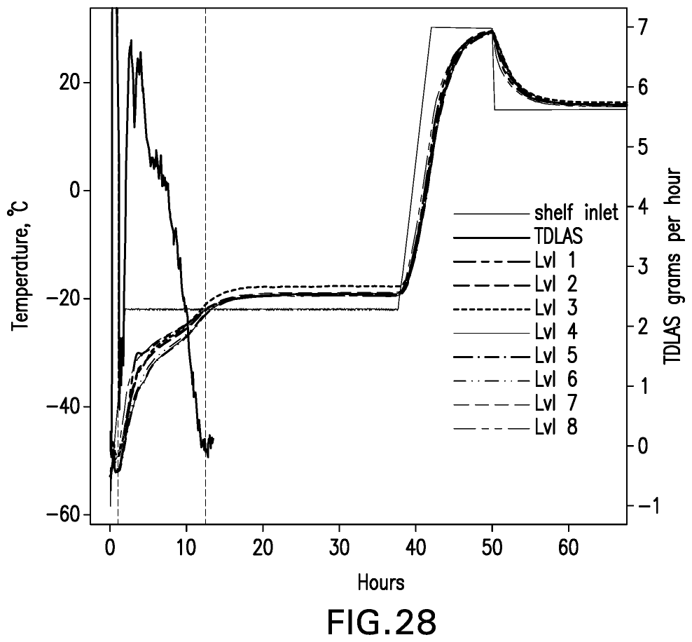

The hold time at −22° C. was intentionally much longer than needed for primary drying, as an experiment to determine how long it would take to complete primary drying of these particular formulations at −22° C. During lyophilization, each of the eight stacked assemblies had two thermocouples firmly taped to both sides of the front face of the base plates. The average of the two thermocouples on each plate was taken as the temperature for that base plate and provides information about temperature uniformity of the base plates over time. FIG. 28 shows thermocouple temperatures during lyophilization, the shelf inlet temperature, and TDLAS data showing mass of removed water, all as a function of time. As seen in FIG. 28, primary drying at −22° C. was complete within about 12 hours. It is expected that the lyophilization program can be further optimized.

After drying, the carrier with the stack of assemblies with lyobeads was removed from the lyophilizer into a glovebox with a predominantly dry nitrogen atmosphere. The lyobeads were dispensed into vials (as explained below) and had very good visual appearance.

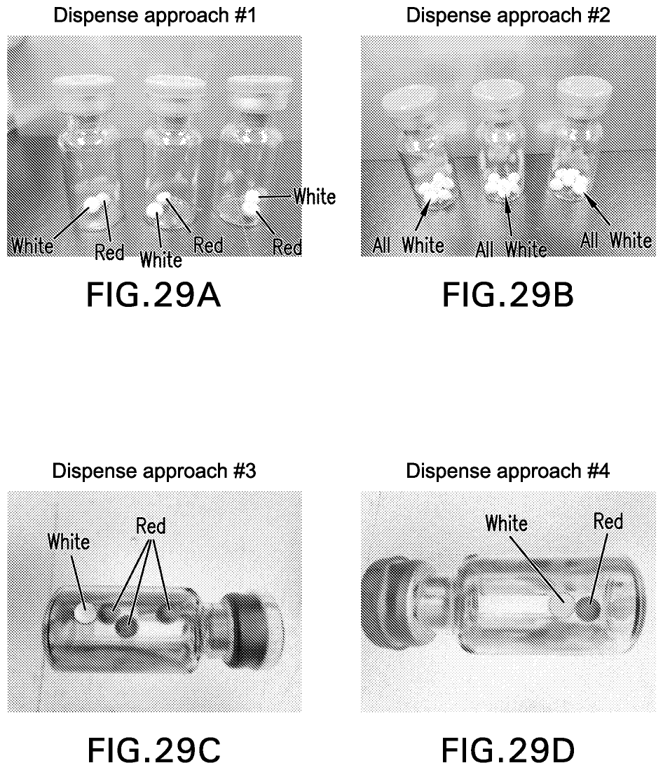

Several demonstrations of different useful ways to dispense the lyobeads using combiner and accumulator dispensing layers were performed. FIGS. 29A-29D show resulting vials from each of four dispense approaches.

### Dispense Approach 1

One assembly of lyobeads (1×100 lyobeads) was dispensed into an array of 50 vials (2 lyobeads per vial) using a combiner dispensing funnel layer. The assembly had alternating rows of white lyobeads and red lyobeads, resulting in vials that had one red lyobead and one white lyobead (FIG. 29A). This demonstrated that lyobeads of different formulations, frozen and lyophilized on the same assembly base plate, can be dispensed into vials resulting in combination drug product vials with multiple different lyobead formulations in the same vial.

### Dispense Approach 2

Three assemblies of lyobeads (3×100 lyobeads), one after another, were dispensed into an array of 50 vials (6 lyobeads per vial) using a combiner dispensing funnel layer. The three assemblies had all white lyobeads. FIG. 29B shows that each vial contained 6 white lyobeads after dispensing of three assemblies one after another.

### Dispense Approach 3

Two assemblies of lyobeads (2×100 lyobeads), one after the other, were dispensed into an array of 50 vials (4 lyobeads per vial) using a combiner dispensing funnel layer. One assembly had alternating rows of white lyobeads and red lyobeads, and the other assembly had all red lyobeads, resulting in vials that had three red lyobeads and one white lyobead (FIG. 29C).

This demonstrated that a combiner dispensing funnel layer can be used in many ways with arrays of lyobeads to achieve different desired combinations and ratios of lyobeads in final containers.

### Dispense Approach 4

Two assemblies of lyobeads (2×100 lyobeads), one after the other, were dispensed into the accumulator dispensing layer with the lower slide plate “closed” (solid plate beneath the lyobead accumulation space) so that lyobeads were retained within the accumulator dispensing layer for a period of time before subsequent dispense into vials. One of the assemblies had all white lyobeads, and the other assembly had all red lyobeads, resulting in one red lyobead and one white lyobead in each of 100 chute positions within the accumulator. The accumulator layer also had a lid that was used during the storage time. The accumulator layer with 2 lyobeads per array position (200 lyobeads total) was placed on a nest of 100 vials, and the lower slide plate moved slightly to allow the lyobeads to drop into the vials (2 lyobeads in each of 100 vials, one red and one white).

### Example 7. Preparing and Dispensing Lyospheres Using an Adapter Dispensing Funnel

Three different dispensing funnels were used in Example 7, one of which differs from the dispensing funnels used in Examples 5 and 6. This different dispensing funnel used in Example 7 has the property of directing an array of lyobeads into an array of fillable syringes. Furthermore, in this Example 7, the dimensions of the assembly (interfacing on the top surface of the dispensing funnel) were different than the dimensions of the syringe array (interfacing on the lower surface of the dispensing funnel). This dispensing funnel that directed lyobeads into an array of syringes also changed the physical dimensions of the array. This dispensing funnel is referred as an adapter dispensing funnel, adapting between arrays in which at least some of the openings are in different relative locations, comparing the array on the top surface (which interfaces with an assembly) and the array on the lower surface (which interfaces with a syringe or container nest with different dimensions). A useful feature of such an adapter dispensing funnel is that the same assemblies can be used for eventual dispense into either nests of vials or nests of syringes even if the nests of final containers have different dimensions for their arrays. Images that illustrate the concept of this adapter dispensing funnel are shown in FIGS. 35A-35H. Note that for this example the drop channels in the central region of the adapter dispensing layer are vertical or nearly vertical, while the drop channels on either side of the adapter dispensing funnel are curved and/or slanted toward the center of the adapter dispensing funnel, thus reducing the length of the array in one dimension.

One hundred microliter liquid droplets were frozen then lyophilized. The following final containers of lyobeads were prepared: 100 syringes, each with 5 lyobeads; 50 vials, each with 1 lyobead; and 48 vials, each with 5 lyobeads.

Assemblies were pre-cooled in a −70° C. freezer overnight prior to use for freezing lyobeads. Heat sinks packed in a tray of dry ice were blast frozen at −115° C., following which pre-cooled assemblies were removed from the −70° C. freezer and placed on top of the chilled heat sinks. Base plates were not physically attached to the heat sink. 100 microliter droplets of a formulation were dispensed on the base plates in an array format, and the droplets froze on the base plates. Assemblies with frozen droplets in an array were placed back in the −70° C. freezer until the start of the lyophilization cycle. The formulation used in this example has a very low Tg′ (glass transition temperature for the frozen liquid) below about −40° C.

The base plates with frozen droplets were stacked one above another with a thermally conductive path formed between the assemblies, and the stack of eight assemblies (held within a carrier) was placed in a lyophilizer. The frozen droplets were lyophilized on the same base plates on which they were frozen, in an array format, with assemblies stacked one above another. The stoppering function of the lyophilizer was used to move the lyophilizer shelves together, so that the stack of assemblies was compressed between a lyophilizer shelf from below and a lyophilizer shelf from above.

The lyophilization drying program used for this example is shown in Table 7.

TABLE 7
Lyophilization drying program

| Step | 1 | 2 | 3 | 4 |
|---|---|---|---|---|
| Shelf Setpoint, ° C. | −50 | −25 | +30 | +15 |
| Ramp Rate, ° C./min | (initial) | 0.1 | 0.2 | 1 |
| Hold Time, min | 1 | 2160 | 360 | (final hold) |
| Vacuum, mTorr | 30 | 30 | 30 | 30 |

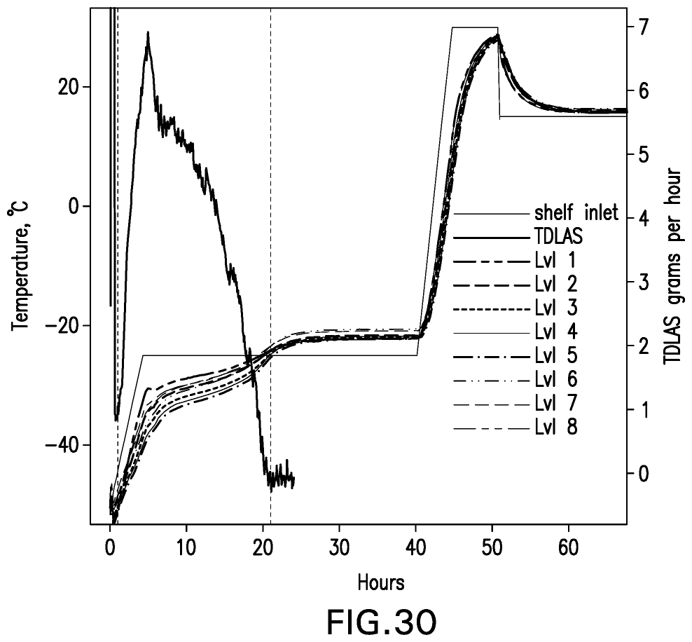

The hold time at −25° C. was intentionally much longer than needed for primary drying, as an experiment to determine how long it would take to complete primary drying of these particular lyobeads at −25° C. During lyophilization, each of the eight stacked assemblies had two thermocouples firmly taped to both sides of the front face of the base plates. The average of the two thermocouples on each plate was taken as the temperature for that base plate and provides information about temperature uniformity of the base plates over time. FIG. 30 shows thermocouple temperatures during lyophilization, the shelf inlet temperature, and TDLAS data showing mass of removed water, all as a function of time. As seen in FIG. 30, primary drying at −25° C. was complete within about 21 hours. It is expected that the lyophilization program can be further optimized.

After drying, the carrier with the stack of assemblies with lyobeads was removed from the lyophilizer into a glovebox with a predominantly dry nitrogen atmosphere. The lyobeads were dispensed into syringes and vials (as explained below) and had very good visual appearance.

### Dispense into Syringes

Five assemblies of lyobeads (5×100 lyobeads) were dispensed into an array of 100 syringes (5 lyobeads per syringe) (BD Hypak SCF 1.5 mL glass syringes) using an adapter dispensing funnel. Syringes were stoppered in a glovebox (predominantly dry nitrogen environment). All lyobeads from the 5 assemblies went into their intended syringes, resulting in 100 out of 100 correct syringes (confirmed by post dispense visual count in each syringe). The time it took to dispense 5×100-microliter lyobeads into each of the 100 syringes in the syringe nest was less than 2 minutes, but dispense speed could have been increased significantly beyond that. It is noted that the lyobead dispense process into syringes by this method was robust despite slight variation in lyobead shape of the manually frozen 100-microliter lyobeads.

### Dispense into Vials

In addition to dispensing into syringes, lyobeads were also dispensed into vials. 100 vials were placed into a nest array (with different dimensions than the syringe array described above). Three assemblies of lyobeads (3×100 lyobeads), one after another, were dispensed into the vials. The first assembly of 100 lyobeads was dispensed using a straight through dispensing funnel, to direct 1 lyobead into each of the 100 vials. The second and third assemblies were dispensed using a combiner dispensing funnel (described in more detail in Example 6), which directed 2 lyobeads each into 50 vials. The intended result of this process was to result in 50 vials in the nested array that had 1 lyobead, and 50 vials in the nested array that had 5 lyobeads. This also demonstrates that the same set of assemblies can be used to dispense into container arrays with different dimensions (for example, syringe nests and vial nests) by use of different dispensing funnels.

### Example 8. Preparing and Dispensing Lyospheres of a Vaccine Formulation and an Adjuvant Formulation Using a Combiner Dispensing Funnel

A vaccine formulation and an adjuvant formulation were mixed together shortly before dispensing and freezing the droplets, due to perceived risk of instability in the liquid phase when the vaccine and the adjuvant were co-formulated. In this example, 100 microliters of liquid droplets were frozen then lyophilized. The final containers of lyobeads comprised 100 syringes, each with 5 lyobeads and 50 vials, each with 2 lyobeads.

Assemblies were pre-cooled in a −70° C. freezer overnight prior to use for freezing lyobeads. Heat sinks packed in a tray of dry ice were blast frozen at −115° C., following which pre-cooled assemblies were removed from the −70° C. freezer and placed on top of the chilled heat sinks. Base plates were not physically attached to the heat sink. 100 microliter droplets of the mixture of the vaccine formulation and the adjuvant formulation were dispensed on the base plates in an array format, and the droplets froze on the base plates. Assemblies with frozen beads in an array were placed back in the −70° C. freezer until the start of the lyophilization cycle. The base plates with frozen droplets were stacked one above another with a thermally conductive path formed between the assemblies, and the stack of eight assemblies (held within a carrier) was placed in a lyophilizer. The frozen droplets were lyophilized on the same base plates on which they were frozen, in an array format, with assemblies stacked one above another. The stoppering function of the lyophilizer was used to move the lyophilizer shelves together, so that the stack of assemblies was compressed between a lyophilizer shelf from below and a lyophilizer shelf from above.

The lyophilization drying program used for this example is shown in Table 8.

TABLE 8
Lyophilization drying program

| Step | 1 | 2 | 3 | 4 |
|---|---|---|---|---|
| Shelf Setpoint, ° C. | −50 | −20 | +35 | +15 |
| Ramp Rate, ° C./min | (initial) | 0.1 | 0.3 | 1 |
| Hold Time, min | 1 | 2100 | 300 | (final hold) |
| Vacuum, mTorr | 30 | 30 | 30 | 30 |

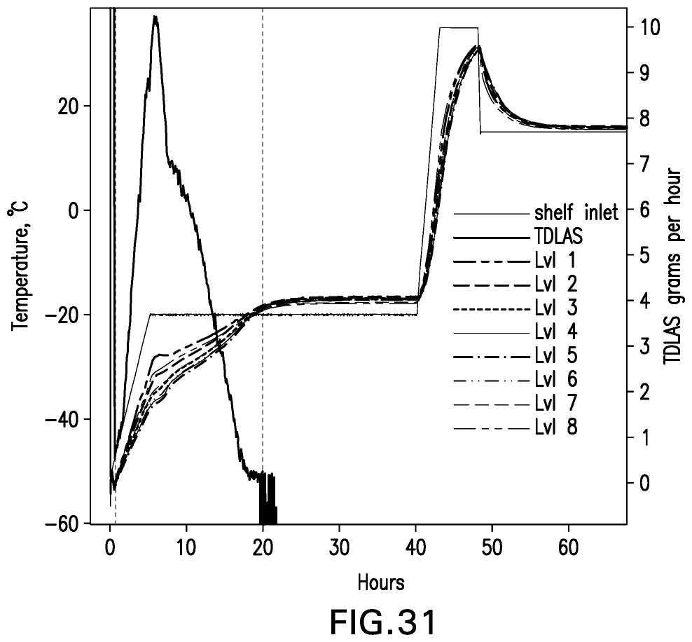

The hold time at −20° C. was intentionally much longer than needed for primary drying, as an experiment to determine how long it would take to complete primary drying of these particular lyobeads at −20° C. During lyophilization, each of the eight stacked assemblies had two thermocouples firmly taped to both sides of the front face of the base plates. The average of the two thermocouples on each plate was taken as the temperature for that base plate and provides information about temperature uniformity of the base plates over time. FIG. 31 shows thermocouple temperatures during lyophilization, the shelf inlet temperature, and TDLAS data showing mass of removed water, all as a function of time. As seen in FIG. 31, primary drying at −20° C. was complete within about 20-21 hours. It is expected that the lyophilization program can be further optimized.

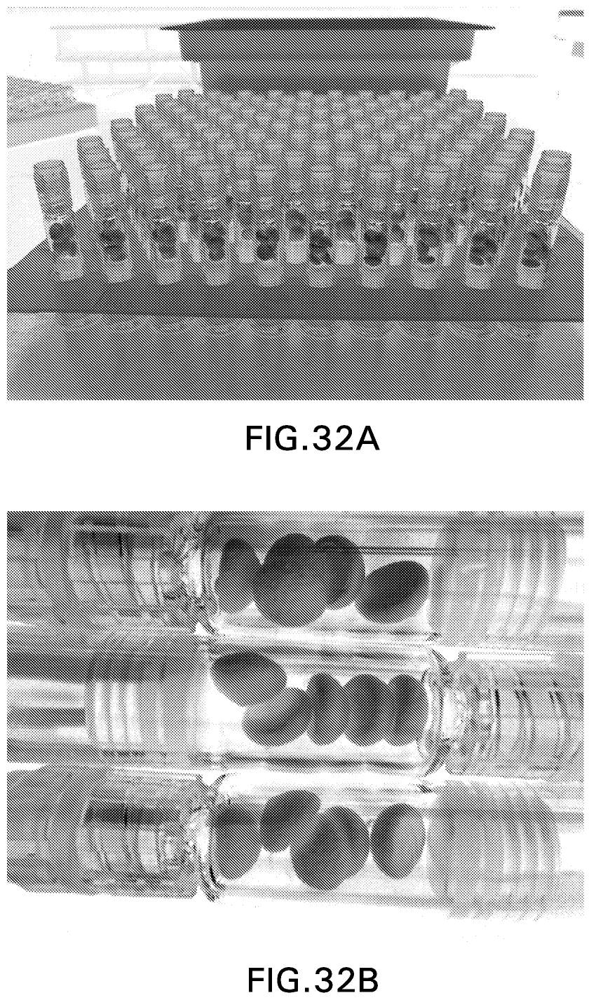

After drying, the carrier with the stack of assemblies with lyobeads was removed from the lyophilizer into a glovebox with a predominantly dry nitrogen atmosphere. The lyobeads were dispensed into syringes and vials (as explained below) and had good visual appearance as shown in FIG. 32.

### Dispense into Syringes

Five assemblies of lyobeads (5×100 lyobeads) were dispensed into an array of 100 syringes (5 lyobeads per syringe) (BD Hypak SCF 1.5 mL glass syringes) using an adapter dispensing funnel. Syringes were stoppered in a glovebox (predominantly dry nitrogen environment). The time it took to dispense 5×100-microliter lyobeads into each of the 100 syringes in the syringe nest was less than about 1.5 minutes, but dispense speed could have been increased significantly beyond that. It is noted that the lyobead dispense process into syringes by this method was robust despite slight variation in lyobead shape of the manually frozen 100-microliter lyobeads.

### Dispense into Vials

In addition to dispensing into syringes, lyobeads were also dispensed into vials. 50 vials were placed into a nest array (with different dimensions than the syringe array described above). One assembly of lyobeads (1×100 lyobeads) was dispensed into the vials using a combiner dispensing funnel (described in more detail in Example 6), which directed 2 lyobeads each into 50 vials. The intended result of this process was to result in 50 vials in the nested array that had 2 lyobeads each. This demonstrates the successful use of a combiner dispensing funnel. This also demonstrates that the same set of assemblies can be used to dispense into container arrays with different dimensions (for example, syringe nests and vial nests) by use of different dispensing funnels.

### Example 9 Preparing and Dispensing lyospheres Using a Clip-Style Assembly

Example 9 describes and demonstrates a clip-style assembly that is different than the assembly shown in FIG. 6. This alternate assembly comprises a planar base plate (in this example, of stainless steel) and a planar insert plate (in this example, of plastic or delrin). It also comprises thermally conductive clips (in this example, of aluminum) on two of the sides that hold the base plate and the insert plate together. These assemblies are stackable, and when stacked will be in physical contact with each other through the thermally conductive clips. If the lowest assembly of such a stack of these assemblies were placed on a lyophilizer shelf, the lowest base plate would not be in full contact with the lyophilizer shelf, rather the contact would be with the clips along the edges.

This assembly can be fabricated using methods that are cost effective at larger scales, including perforation punching. The plastic plate can be manufactured by a variety of means such as perforation punching or injection molding. The base plate and insert plate can also be fabricated by other methods such as machining and/or cutting, and by combinations of methods. In this example, a combination of perforation punching and machining were employed to fabricate the base plate and insert plate.

The clips have two approximately parallel planar surfaces, which are useful for stacking assemblies one on top of another. Those two planar surfaces are connected by a portion between them that may comprise an arc or radius of curvature and may present the general appearance of a “U” shape when viewed on end. The clips can be fabricated for example from sheet metal by means known in the art.

FIG. 36A shows a photograph of one base plate (of stainless steel), one insert plate (of delrin plastic) and two clips (of aluminum), before the clips have been applied to hold the base plate and the insert plate together. FIG. 36B shows the same metal base plate and plastic insert plate with the metal clips applied (thus holding the base plate and insert plate together). Furthermore, FIG. 36B shows this assembly resting on top of an adapter dispensing funnel, which in turn is resting upon pre-fillable syringes within a nest and tub of syringes. To demonstrate lyobead dispense from this assembly, one glass bead was placed in each of the 100 circular holes through the insert plate, with the insert plate and base plate clipped together and aligned relative to each other such that the glass beads rested upon the solid portions of the base plate, as shown in FIG. 37A. The adapter dispensing funnel on which the assembly with beads rested was constructed so that the handle portion of the plastic insert plate could be displaced relative to the metal base plate, while the metal base plate was held in position. The white dotted-line circles overlaid on top of the photographs in FIG. 37A highlight design features of the adapter dispensing funnel that hold the metal base plate in place and the metal clips in place while allowing the plastic insert plate to slide when the handle is pulled. Pulling the handle of the plastic insert plate results in alignment of the holes in the insert plate and the holes in the metal base plate, and beads drop through the adapter dispensing funnel and into the syringes below (FIG. 37B). Briefly, the adapter feature of this dispensing funnel (explained more thoroughly in previous examples) is that the array of 100 beads has different dimensions and positions than the array of 100 syringe openings below (into which the beads drop), so the funnel directs the beads in this case from an array that is different (in this case larger) in at least 1 dimension, into an array that is different (in this case smaller in at least 1 dimension). With this dispensing funnel, the plastic plate can only be pulled a short distance until it, like the metal base plate, physically runs into the dispensing funnel and is stopped. While this is not a required feature, it seems helpful in not “overshooting” the pull of the plastic insert plate too far, passing the holes in the metal base plate too quickly.

Pulling the plastic plate resulted in the nearly simultaneous drop of 100 beads into 100 syringes below. All the beads (100 out of 100) were properly directed to their target syringes. A photograph of one of the syringes with the dispensed glass bead is shown in FIG. 37C.

What is claimed:

1. (canceled)

2. A method for preparing lyospheres of a pharmaceutical composition, comprising:
(a) providing an assembly comprising a generally planar base plate, wherein the base plate is placed on top of a heat sink and chilled to a low temperature, wherein the base plate is not physically attached to the heat sink;
(b) dispensing droplets of the pharmaceutical composition on the base plate in an array format, wherein the droplets freeze on the base plate; and
(c) placing the assembly in a lyophilizer to dry the frozen droplets and produce an array of lyospheres.

3. The method of claim 2, repeating steps (a) and (b) multiple times, preparing a stack of assemblies with a thermally conductive path formed between the assemblies; and in step (c) drying the frozen droplets in the entire stack in a lyophilizer to produce arrays of lyospheres.

4. The method of claim 3, wherein the assembly further comprises an insert plate overlaying the base plate; wherein the insert plate has an array of apertures; and wherein each droplet is dispensed into an aperture to be supported by the base plate.

5. The method of claim 4, wherein the base plate has an array of openings and solid portions located between and surrounding the openings; wherein the apertures align with the solid portions of the base plate with no overlap with the openings; and wherein each droplet is dispensed into an aperture to be supported by the solid portions of the base plate.

6. A method for preparing lyospheres of a pharmaceutical composition, comprising:
(a) providing an assembly comprising an insert plate overlaying a base plate; wherein the base plate has an array of openings and solid portions located between and surrounding the openings; wherein the insert plate has an array of apertures; wherein the insert plate is axially shiftable relative to the base plate from a first state to a second state; wherein in the first state, the apertures align with the solid portions of the base plate with no overlap with the openings, and in the second state, the apertures at least partially align with the openings so as to at least partially overlap with the openings; wherein the base plate is chilled to a low temperature;
(b) while the insert plate is in the first state, dispensing droplets of the pharmaceutical composition into the apertures to be supported by the solid portions of the base plate, wherein the droplets freeze on the base plate;
(c) placing the assembly in a lyophilizer to dry the frozen droplets and produce lyospheres;
(d) providing a container nest comprising an array of pharmaceutically acceptable containers, each of the containers having a loading opening, wherein the openings in the base plate align with the loading openings of the containers; and
(e) dispensing the lyospheres into the containers by axially shifting the insert plate relative to the base plate to the second state.

7. The method of claim 6, repeating steps (a)-(b) multiple times, preparing a stack of assemblies with a thermally conductive path formed between the assemblies; in step (c) drying the frozen droplets in the entire stack in a lyophilizer to produce lyospheres; then repeating steps (d)-(e) multiple times to dispense the lyospheres in each assembly into the containers in a container nest.

8. The method of claim 3, wherein the thermally conductive path is formed by stacking a plurality of assemblies on top of each other, wherein each base plate has at least two raised edges, and wherein the raised edges of the base plates are in physical contact.

9. The method of claim 3, wherein the thermally conductive path is formed by placing thermally conductive spacers along at least two edges of each base plate and stacking a plurality of assemblies on top of each other, wherein the spacers and the base plates are in physical contact.

10. The method of claim 3, wherein the thermally conductive path is formed by providing a thermally conductive rack with multiple levels and placing a plurality of assemblies on the levels of the rack, wherein the base plates and the levels of the rack are in physical contact.

11. The method of claim 3, wherein the thermally conductive path is formed by edge mounting two thermally conductive clips to the base plate and the insert plate with the insert plate overlaying the base plate and stacking a plurality of assemblies on top of each other, wherein the clips are in physical contact.

12. The method of claim 3, wherein in step (c) the lowest base plate is not in full contact with a shelf of the lyophilizer.

13. The method of claim 6, wherein the dispensing droplets of the pharmaceutical composition is at a speed of: from about 0.5 mL/min to about 75 mL/min, from about 0.5 mL/min to about 50 mL/min, from about 5 mL/min to about 50 mL/min, from about 5 mL/min to about 40 mL/min, from about 10 mL/min to about 40 mL/min, or from about 10 mL/min to about 30 mL/min.

14. The method of claim 6, wherein the droplet is about 10, 15, 20, 25, 30, 40, 50, 75, 100, 125, 150, 175, 200, 225, or 250 μL.

15. The method of claim 6, wherein the dispensing droplets of the pharmaceutical composition is through a dispensing tip; wherein the distance from the bottom of the dispensing tip to the base plate is: from about 0.05 cm to about 1 cm, from about 0.05 cm to about 0.8 cm, from about 0.05 cm to about 0.5 cm, from about 0.05 cm to about 0.3 cm, or from about 0.1 cm to about 0.3 cm.

16. The method of claim 6, wherein the temperature of the base plate is: from about −70° C. to about −196° C., from about −70° C. to about −150° C., from about −90° C. to about −196° C., from about −150° C. to about −196° C., from about −180° C. to about −196° C., or from about −180° C. to about −273° C.

17. The method of claim 6, wherein the pharmaceutical composition comprises a drug substance, a chemical compound, a therapeutic protein, an antibody, a vaccine, a fusion protein, a polypeptide, a peptide, a polynucleotide, a nucleotide, an antisense RNA, a siRNA, an oncolytic virus, a diagnostic, an enzyme, an adjuvant, an antigen, a virus, a virus-like particle, a prodrug, a toxoid, a vitamin, a lipid, a lipid nanoparticle, or a combination thereof.

18. The method of claim 6, wherein each container further comprises a lyosphere of a second pharmaceutical composition.

19. (canceled)

20. An assembly for preparing lyospheres, the assembly comprising:
a base plate having a generally planar base with an array of openings formed therethrough, solid portions of the base being located between, and surrounding, the openings; and,
an insert plate for overlaying the base plate, the insert plate having a generally planar body with an array of apertures formed therethrough, the insert plate being axially shiftable relative to the base plate from a first state to a second state, wherein, the array of apertures is configured such that, in the first state, the apertures are aligned with the solid portions of the base plate with no overlap with the openings, and, in the second state, the apertures are at least partially aligned with the openings so as to at least partially overlap the openings.

21. The assembly of claim 20, wherein the base plate includes spaced-apart first and second side edges which extend between spaced-apart first and second ends, optionally the first and second side edges each being greater in length than each of the first and second ends.

22. The assembly of claim 21, wherein the base plate further comprises a first upstanding channel extending along the first side edge and a second upstanding channel extending along the second side edge, the first and second channels being configured to receive the insert plate in sliding engagement to guide the insert plate during the axial shifting of the insert plate relative to the base plate.

23. The assembly of claim 22, wherein the first channel includes a first wall extending upwardly from the first side edge and a second wall extending transversely from the first wall spaced from, in overlapping relation to, the base, wherein the second channel includes a third wall extending upwardly from the second side edge and a fourth wall extending transversely from the third wall spaced from, in overlapping relation to, the base, and, wherein the second wall defines an upper surface generally coplanar to an upper surface defined by the fourth wall so as to define a common resting surface therewith.

24. The assembly of claim 20, further comprising first and second clips edge mounted to the base plate and the insert plate with the insert plate overlaying the base plate.

25. The assembly of claim 20, wherein the apertures are all similarly formed, wherein the openings are all similarly formed, and, wherein a first of the apertures defines an open area larger than an open area defined by a first of the openings.

26. The assembly of claim 20, wherein the apertures are each generally circular, and the openings are each generally oval shaped.

27. The assembly of claim 26, wherein the openings are each elongated along a longitudinal axis, a first of the openings defining a first length along the respective longitudinal axis, the first length being generally equal to a diameter of a first of the apertures.

28. The assembly of claim 27, wherein, with the insert plate in the second state, the diameter of the first aperture is generally coaxial with the longitudinal axis of the first opening.

29-65. (canceled)
